SEQUENCE LISTING (49 sequences)

SEQ ID NO: 1 - protein, 33 aa, JC virus
MDKVLNREESMELMDLLGLDRSAWGNIPVMRKL

SEQ ID NO: 2 - protein, 240 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 3 - protein, 556 aa, Homo sapiens [sequence omitted]

SEQ ID NO: 4 - protein, 847 aa, Homo sapiens
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 5 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 6 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 7 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 8 - protein, 858 aa, Homo sapiens
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKASWTRPEKQETLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVQGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPANGSPTSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 9 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 10 - protein, 1091 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQGQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPGEWLVWKQSCSSTSSTHSAAASLQCPSQVLPPASPEGETVADLQTQ

SEQ ID NO: 11 - protein, 1255 aa, Homo sapiens [sequence omitted]

SEQ ID NO: 12 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 13 - protein, 427 aa, Homo sapiens
MEWPARLCGLWALLLCAGGGGGGGGAAPTETQPPVTNLSVSVENLCTVIWTWNPPEGASSNCSLWYFSHFGDKQDKKIAPETRRSIEVPLNERICLQVGSQCSTNESEKPSILVEKCISPPEGDPESAVTELQCIWHNLSYMKCSWLPGRNTSPDTNYTLYYWHRSLEKIHQCENIFREGQYFGCSFDLTKVKDSSFEQHSVQIMVKDNAGKIKPSFNIVPLTSRVKPDPPHIKNLSFHNDDLYVQWENPQNFISRCLFYEVEVNNSQTETHNVFYVQEAKCENPEFERNVENTSCFMVPGVLPDTLNTVRIRVKTNKLCYEDDKLWSNWSQEMSIGKKRNSTLYITMLLIVPVIVAGAIIVLLLYLKRLKIIIFPPIPDPGKIFKEMFGDQNDDTLHWKKYDIYEKQTKEETDSVVLIENLKKASQ

SEQ ID NO: 14 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 15 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 16 - protein, 19 aa, Artificial Sequence
  chemically synthesized
TFFYGGCRGKRNNFKTEEY

SEQ ID NO: 17 - protein, 19 aa, Artificial Sequence
  chemically synthesized
TFFYGGSRGKRNNFKTEEY

SEQ ID NO: 18 - protein, 19 aa, Artificial Sequence
  chemically synthesized
TFFYGGSRGKRNNFRTEEY

SEQ ID NO: 19 - protein, 19 aa, Artificial Sequence
  chemically synthesized
TFFYGGSRGRRNNFRTEEY

SEQ ID NO: 20 - protein, 19 aa, Artificial Sequence
  chemically synthesized
TFFYGGCRAKRNNFKRAKY

SEQ ID NO: 21 - protein, 19 aa, Artificial Sequence
  chemically synthesized
TFFYGGCRGKKNNFKRAKY

SEQ ID NO: 22 - protein, 19 aa, Artificial Sequence
  chemically synthesized
PFFYGGCRGKRNNFKTEEY

SEQ ID NO: 23 - protein, 19 aa, Artificial Sequence
  chemically synthesized
TFFYGGCRGKRNNFKTKEY

SEQ ID NO: 24 - protein, 19 aa, Artificial Sequence
  chemically synthesized
TFFYGGKRGKRNNFKTKEY

SEQ ID NO: 25 - protein, 19 aa, Artificial Sequence
  chemically synthesized
TFFYGGCRGKRNNFKTKRY

SEQ ID NO: 26 - protein, 19 aa, Artificial Sequence
  chemically synthesized
TFFYGGKRGKRNNFKTAEY

SEQ ID NO: 27 - protein, 19 aa, Artificial Sequence
  chemically synthesized
TFFYGGKRGKRNNFKREKY

SEQ ID NO: 28 - protein, 19 aa, Artificial Sequence
  chemically synthesized
RFKYGGCLGNKNNFLRLKY

SEQ ID NO: 29 - protein, 19 aa, Artificial Sequence
  chemically synthesized
RFKYGGCLGNKNNYLRLKY

SEQ ID NO: 30 - protein, 353 aa, Human T-cell lymphotropic virus
MAHFPGFGQSLLFGYPVYVFGDCVQGDWCPISGGLCSARLHRHALLATCPEHQITWDPIDGRVIGSALQFLIPRLPSFPTQRTSKTLKVLTPPITHTTPNIPPSFLQAMRKYSPFRNGYMEPTLGQHLPTLSFPDPGLRPQNLYTLWGGSVVCMYLYQLSPPITWPLLPHVIFCHPGQLGAFLTNVPYKRIEKLLYKISLTTGALIILPEDCLPTTLFQPARAPVTLTAWQNGLLPFHSTLTTPGLIWTFTDGTPMISGPCPKDGQPSLVLQSSSFIFHKFQTKAYHPSFLLSHGLIQYSSFHNLHLLFEEYTNIPISLLFNEKEADDNDHEPQISPGGLEPLSEKHFRETEV

SEQ ID NO: 31 - protein, 380 aa, Homo sapiens
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 32 - protein, 14 aa, Artificial Sequence
  chemically synthesized
GRKKRRQRRRGYXC

SEQ ID NO: 33 - protein, 1382 aa, Homo sapiens [sequence omitted]

SEQ ID NO: 34 - DNA, 1656 bp, Homo sapiens
tagacccagagaggctcagctgcactcgcccggctgggagagctgggtgtggggaacatggccgggcctccgaggctcctgctgctgcccctgcttctggcgctggctcgcggcctgcctggggccctggctgcccaaggtaagagcttcccaggctctccatggccacagctccggagctctccctgccccatgagctcagagcccccagtctgagccacagcacagcccccaggaagcgggtggggtgctgagcggcctccagtgtctgaggactcatttaagagaaggaaaaagggtggacccggtggggagtggccggggctgtccaggcagggccgctgctttgggaggaagaagcccacagtctcggaacacgaggacagcacctcccccaacaccacagccggtgcccagatctgctccatgccccgtaaggcaccgtgtctttggcgacatgtcagccctgggctgtctcagggccccaccatccccaccactgtcccctgcagggaggacattctctgtccttctggccagactgatggtgacagcccaggtcctcccagaggtgcagcagtctccccactgcacgactgtccccgtgggagcctccgtcaacatcacctgctccaccagcgggggcctgcgtgggatctacctgaggcagctcgggccacagccccaagacatcatttactacgaggacggggtggtgcccactacggacagacggttccggggccgcatcgacttctcagggtcccaggacaacctgactatcaccatgcaccgcctgcagctgtcggacactggcacctacacctgccaggccatcacggaggtcaatgtctacggctccggcaccctggtcctggtgacagaggaacagtcccaaggatggcacagatgctcggacgccccaccaagggcctctgccctccctgccccaccgacaggctccgccctccctgacccgcagacagcctctgccctccctgacccgccagcagcctctgccctccctgcggccctggcggtgatctccttcctcctcgggctgggcctgggggtggcgtgtgtgctggcgaggacacagataaagaaactgtgctcgtggcgggataagaattcggcggcatgtgtggtgtacgaggacatgtcgcacagccgctgcaacacgctgtcctcccccaaccagtaccagtgacccagtgggcccctgcacgtcccgcctgtggtccccccagcaccttccctgccccaccatgccccccaccctgccacacccctcaccctgctgtcctcccacggctgcagcagagtttgaagggcccagccgtgcccagctccaagcagacacacaggcagtggccaggccccacggtgcttctcagtggacaatgatgcctcctccgggaagccttccctgcccagcccacgccgccaccgggaggaagcctgactgtcctttggctgcatctcccgaccatggccaaggagggcttttctgtgggatgggcctggcacgcggccctctcctgtcagtgccggcccacccaccagcaggcccccaacccccaggcagcccggcagaggacgggaggagaccagtcccccacccagccgtaccagaaataaaggcttctgtgcttc

SEQ ID NO: 35 - DNA, 1966 bp, Homo sapiens
aggcccctgcctgccccagcatcccctgcgcgaagctgggtgccccggagagtctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgatcctcaggtggccagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaaaaaaaaaaaaaaaaaa

SEQ ID NO: 36 - DNA, 2116 bp, Homo sapiens
acgcggaaacaggcttgcacccagacacgacaccatgcatctcctcggcccctggctcctgctcctggttctagaatacttggctttctctgactcaagtaaatgggtttttgagcaccctgaaaccctctacgcctgggagggggcctgcgtctggatcccctgcacctacagagccctagatggtgacctggaaagcttcatcctgttccacaatcctgagtataacaagaacacctcgaagtttgatgggacaagactctatgaaagcacaaaggatgggaaggttccttctgagcagaaaagggtgcaattcctgggagacaagaataagaactgcacactgagtatccacccggtgcacctcaatgacagtggtcagctggggctgaggatggagtccaagactgagaaatggatggaacgaatacacctcaatgtctctgaaaggccttttccacctcatatccagctccctccagaaattcaagagtcccaggaagtcactctgacctgcttgctgaatttctcctgctatgggtatccgatccaattgcagtggctcctagagggggttccaatgaggcaggctgctgtcacctcgacctccttgaccatcaagtctgtcttcacccggagcgagctcaagttctccccacagtggagtcaccatgggaagattgtgacctgccagcttcaggatgcagatgggaagttcctctccaatgacacggtgcagctgaacgtgaagcatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgagtggctgtgggactcgggtcctgcctcgccatcctcatcctggcaatctgtgggctcaagctccagcgacgttggaagaggacacagagccagcaggggcttcaggagaattccagcggccagagcttctttgtgaggaataaaaaggttagaagggcccccctctctgaaggcccccactccctgggatgctacaatccaatgatggaagatggcattagctacaccaccctgcgctttcccgagatgaacataccacgaactggagatgcagagtcctcagagatgcagagacctcccccggactgcgatgacacggtcacttattcagcattgcacaagcgccaagtgggcactatgagaacgtcattccagattttccagaagatgaggggattcattactcagagctgatccagtttggggtcggggagcggcctcaggcacaagaaaatgtggactatgtgatcctcaaacattgacactggatgggctgcagcagaggcactgggggcagcgggggccagggaagtccccgagttttccccagac

SEQ ID NO: 37 - DNA, 3216 bp, Homo sapiens
ggcagtttcctggctgaacacgccagcccaatacttaaagagagcaactcctgactccgatagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctagaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcattaaagcatgaatggtatggaactctctccaccctatatgtagtataaagaaaagtaggtttacattcatctcattccaacttcccagttcaggagtcccaaggaaagccccagcactaacgtaaatacacaacacacacactctaccctatacaactggacattgtctgcgtggttcctttctcagccgcttctgactgctgattctcccgttcacgttgcctaataaacatccttcaagaactctgggctgctacccagaaatcattttacccttggctcaatcctctaagctaacccccttctactgagccttcagtcttgaatttctaaaaaacagaggccatggcagaataatctttgggtaacttcaaaacggggcagccaaacccatgaggcaatgtcaggaacagaaggatgaatgaggtcccaggcagagaatcatacttagcaaagttttacctgtgcgttactaattggcctctttaagagttagtttctttgggattgctatgaatgataccctgaatttggcctgcactaatttgatgtttacaggtggacacacaaggtgcaaatcaatgcgtacgtttcctgagaagtgtctaaaaacaccaaaaagggatccgtacattcaatgtttatgcaaggaaggaaagaaagaaggaagtgaagagggagaagggatggaggtcacactggtagaacgtaaccacggaaaagagcgcatcaggcctggcacggtggctcaggcctataaccccagctccctaggagaccaaggcgggagcatctcttgaggccaggagtttgagaccagcctgggcagcatagcaagacacatccctacaaaaaattagaaattggctggatgtggtggcatacgcctgtagtcctagccactcaggaggctgaggcaggaggattgcttgagcccaggagttcgaggctgcagtcagtcatgatggcaccactgcactccagcctgggcaacagagcaagatcctgtctttaaggaaaaaaagacaagatgagcataccagcagtccttgaacattatcaaaaagttcagcatattagaatcaccgggaggccttgttaaaagagttcgctgggcccatcttcagagtctctgagttgttggtctggaatagagccaaatgttttgtgtgtctaacaattcccaggtgctgttgctgctgctactattccaggaacacactttgagaaccattgtgttattgctctgcacgcccacccactctcaactcccacgaaaaaaatcaacttccagagctaagatttcggtggaagtcctggttccatatctggtgcaagatctcccctcacgaatcagttgagtcaacattctagctcaacaacatcacacgattaacattaacgaaaattattcatttgggaaactatcagccagttttcacttctgaaggggcaggagagtgttatgagaaatcacggcagttttcagcagggtccagattcagattaaataactattttctgtcatttctgtgaccaaccacatacaaacagactcatctgtgcactctccccctcccccttcaggtatatgttttctgagtaaagttgaaaagaatctcagaccagaaaatatagatatatatttaaatcttacttgagtagaactgattacgacttttgggtgttgaggggtctataagatcaaaacttttccatgataatactaagatgttatcgaccatttatctgtccttctctcaaaagtgtatggtggaattttccagaagctatgtgatacgtgatgatgtcatcactctgctgttaacatataataaatttattgctattgtttataaaagaataaatgatatttttt

SEQ ID NO: 38 - DNA, 3686 bp, Homo sapiens
atacgggagaactaaggctgaaacctcggaggaacaaccacttttgaagtgacttcgcggcgtgcgttgggtgcggactaggtggccgcggcgggagtgtgctggagcctgaagtccacgcgcgcggctgagaaccgccgggaccgcacgtgggcgccgcgcgcttcccccgcttcccaggtgggcgccggccgccaggccacctcacgtccggccccggggatgcgcgtcctcctcgccgcgctgggactgctgttcctgggggcgctacgagccttcccacaggatcgacccttcgaggacacctgtcatggaaaccccagccactactatgacaaggctgtcaggaggtgctgttaccgctgccccatggggctgttcccgacacagcagtgcccacagaggcctactgactgcaggaagcagtgtgagcctgactactacctggatgaggccgaccgctgtacagcctgcgtgacttgttctcgagacgacctcgtggagaagacgccgtgtgcatggaactcctcccgtgtctgcgaatgtcgacccggcatgttctgttccacgtctgccgtcaactcctgtgcccgctgcttcttccattctgtctgtccggcagggatgattgtcaagttcccaggcacggcgcagaagaacacggtctgtgagccggcttccccaggggtcagccctgcctgtgccagcccagagaactgcaaggaaccctccagtggcaccatcccccaggccaagcccaccccggtgtccccagcaacctccagtgccagcaccatgcctgtaagagggggcacccgcctcgcccaggaagctgcttctaaactgacgagggctcccgactctccctcctctgtgggaaggcctagttcagatccaggtctgtccccaacacagccatgcccagaggggtctggtgattgcagaaagcagtgtgagcccgactactacctggacgaggccggccgctgcacggcctgcgtgagctgttctcgagatgaccttgtggagaagacgccatgtgcatggaactcctcccgcacctgcgaatgtcgacctggcatgatctgtgccacatcagccaccaactcccgtgcccgctgtgtcccctacccaatctgtgcagcagagacggtcaccaagccccaggatatggctgagaaggacaccacctttgaggcgccacccctggggacccagccggactgcaaccccaccccagagaatggcgaggcgcctgccagcaccagccccactcagagcttgctggtggactcccaggccagtaagacgctgcccatcccaaccagcgctcccgtcgctctctcctccacggggaagcccgttctggatgcagggccagtgctcttctgggtgatcctggtgttggttgtggtggtcggctccagcgccttcctcctgtgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagtgaggcctgggctgggctggggctaggagggcagcagggtggcctctgggaggccaggatggcactgttggcaccgaggttgggggcagaggcccatctggcctgaactgaggctccagcatctagtggtggaccggccggtcactgcaggggtctggtggtctctgcttgcatccccaacttagctgtcccctgacccagagcctaggggatccggggcttgtacagaagagacagtccaaggggactggatcccagcagtgatgttggttgaggcagcaaacagatggcaggatgggcactgccgagaacagcattggtcccagagccctgggcatcagaccttaaccaccaggcccacagcccagcgagggagaggtcgtgaggccagctcccggggcccctgtaaccctactctcctctctccctggacctcagaggtgacacccattgggcccttccggcatgcccccagttactgtaaatgtggcccccagtgggcatggagccagtgcctgtggttgtttctccagagtcaaaagggaagtcgagggatggggcgtcgtcagctggcactgtctctgctgcagcggccacactgtactctgcactggtgtgagggcccctgcctggactgtgggaccctcctggtgctgcccaccttccctgtcctgtagccccctcggtgggcccagggcctagggcccaggatcaagtcactcatctcagaatgtccccaccaatccccgccacagcaggcgcctcgggtcccagatgtctgcagccctcagcagctgcagaccgcccctcaccaacccagagaacctgctttactttgcccagggacttcctccccatgtgaacatggggaacttcgggccctgcctggagtccttgaccgctctctgtgggccccacccactctgtcctgggaaatgaagaagcatcttccttaggtctgccctgcttgcaaatccactagcaccgaccccaccacctggttccggctctgcacgctttggggtgtggatgtcgagaggcaccacggcctcacccaggcatctgctttactctggaccataggaaacaagaccgtttggaggtttcatcaggattttgggtttttcacatttcacgctaaggagtagtggccctgacttccggtcggctggccagctgactccctagggccttcagacgtgtatgcaaatgagtgatggataaggatgagtcttggagttgcgggcagcctggagactcgtggacttaccgcctggaggcaggcccgggaaggctgctgtttactcatcgggcagccacgtgctctctggaggaagtgatagtttctgaaaccgctcagatgttttggggaaagttggagaagccgtggccttgcgagaggtggttacaccagaacctggacattggccagaagaagcttaagtgggcagacactgtttgcccagtgtttgtgcaaggatggagtgggtgtctctgcatcacccacagccgcagctgtaaggcacgctggaaggcacacgcctgccaggcagggcagtctggcgcccatgatgggagggattgacatgtttcaacaaaataatgcacttccttacctagtggcccttcacacaacttttgaatctctaaaaatccataaaatccttaaagaactgtaa

SEQ ID NO: 39 - DNA, 1466 bp, Homo sapiens
tctgctcacacaggaagccctggaagctgcttcctcagacatgccgctgctgctactgctgcccctgctgtgggcaggggccctggctatggatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctatgttccacagaacccaacaactggtatctttccaggagatggctcagggaaacaagagaccagagcaggagtggttcatggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccagtgaggaacccacaagagcatcaggctcagctagaagatccacatcctctacaggtcggggaccaaaggctgattcttggagatttaacaccccacaggcaatgggtttatagacattatgtgagtttcctgctatattaacatcatcttagactttgcaagcagagagtcgtggaatcaaatctgtgctctttcatttgctaagtgtatgatgtcacacaagctccttaaccttccatgtctccattttcttctctgtgaagtaggtataagaagtcctatctcatagggatgctgtgagcattaaataaaggtacacatggaaaacaccagtc

SEQ ID NO: 40 - DNA, 2633 bp, Homo sapiens
ccgcggcaagaacatccctcccagccagcagattacaatgctgcaaactaaggatctcatctggactttgtttttcctgggaactgcagtttctctgcaggtggatattgttcccagccagggggagatcagcgttggagagtccaaattcttcttatgccaagtggcaggagatgccaaagataaagacatctcctggttctcccccaatggagaaaagctcaccccaaaccagcagcggatctcagtggtgtggaatgatgattcctcctccaccctcaccatctataacgccaacatcgacgacgccggcatttacaagtgtgtggttacaggcgaggatggcagtgagtcagaggccaccgtcaacgtgaagatctttcagaagctcatgttcaagaatgcgccaaccccacaggagttccgggagggggaagatgccgtgattgtgtgtgatgtggtcagctccctcccaccaaccatcatctggaaacacaaaggccgagatgtcatcctgaaaaaagatgtccgattcatagtcctgtccaacaactacctgcagatccggggcatcaagaaaacagatgagggcacttatcgctgtgagggcagaatcctggcacggggggagatcaacttcaaggacattcaggtcattgtgaatgtgccacctaccatccgggccaggcagaatattgtgaatgccaccgccaacctcggccagtccgtcaccctggtgtgcgatgccgaacggttcccagagcccaccatgagctggacaaaggatggggaacagatagagcaagaggaagacgatgagaagtacatcttcagcgacgatagttcccagctgaccatcaaaaaggtggataagaacgacgaggctgagtacatctgcattgctgagaacaaggctggcgagcaggatgcgaccatccacctcaaagtctttgcaaaacccaaaatcacatatgtagagaaccagactgccatggaattagaggagcaggtcactcttacctgtgaagcctccggagaccccattccctccatcacctggaggacttctacccggaacatcagcagcgaagaaaagactctggatgggcacatggtggtgcgtagccatgcccgtgtgtcgtcgctgaccctgaagagcatccagtacactgatgccggagagtacatctgcaccgccagcaacaccatcggccaggactcccagtccatgtaccttgaagtgcaatatgccccaaagctacagggccctgtggctgtgtacacttgggaggggaaccaggtgaacatcacctgcgaggtatttgcctatcccagtgccacgatctcatggtttcgggatggccagctgctgccaagctccaattacagcaatatcaagatctacaacaccccctctgccagctatctggaggtgaccccagactctgagaatgattttgggaactacaactgtactgcagtgaaccgcattgggcaggagtccttcgaattcatccttgttcaagcagacaccccctcttcaccatccatcgaccaggtggagccatactccagcacagcccaggtgcagtttgatgaaccagaggccacaggtggggtgcccatcctcaaatacaaagctgagtggagagcagtgggtgaagaagtatggcattccaagtggtatgatgccaaggaagccagcatggagggcatcgtcaccatcgtgggcctgaagcccgaaacaacgtacgccgtaaggctggcggcgctcaatggcaaagggctgggtgagatcagcgcggcctccgagttcaagacgcagccagtccaaggggaacccagtgcacctaagctcgaagggcagatgggagaggatggaaactctattaaagtgaacctgatcaagcaggatgacggcggctcccccatcagacactatctggtcaggtaccgagcgctctcctccgagtggaaaccagagatcaggctcccgtctggcagtgaccacgtcatgctgaagtccctggactggaatgctgagtatgaggtctacgtggtggctgagaaccagcaaggaaaatccaaggcggctcattttgtgttcaggacctcggcccagcccacagccatcccagccaacggcagccccacctcaggcctgagcaccggggccatcgtgggcatcctcatcgtcatcttcgtcctgctcctggtggttgtggacatcacctgctacttcctgaacaagtgtggcctgttcatgtgcattgcggtcaacctgtgtggaaaagccgggcccggggccaagggcaaggacatggaggagggcaaggccgccttctcgaaagatgagtccaaggagcccatcgtggaggttcgaacggaggaggagaggaccccaaaccatgatggagggaaacacacagagcccaacgagaccacgccactgacggagcccgagaagggccccgtagaagcaaagccagagtgccaggagacagaaacgaagccagcgccagccgaagtcaagacggtccccaatgacgccacacagacaaaggagaacgagagcaaagcatgatgggtgaagagaaccgagcaaagatcaaaataaaaagtgacacagcagc

SEQ ID NO: 41 - DNA, 5010 bp, Homo sapiens
ggcggctcgggacggaggacgcgctagtgtgagtgcgggcttctagaactacaccgaccctcgtgtcctcccttcatcctgcggggctggctggagcggccgctccggtgctgtccagcagccatagggagccgcacggggagcgggaaagcggtcgcggccccaggcggggcggccgggatggagcggggccgcgagcctgtggggaaggggctgtggcggcgcctcgagcggctgcaggttcttctgtgtggcagttcagaatgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccagcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcagaaaccagttatgtgaatgatctctctgaatcctaagggctggtctctgctgaaggttgtaagtggttcgcttactttgagtgatcctccaacttcatttgatgctaaataggagataccaggttgaaagacctctccaaatgagatctaagcctttccataaggaatgtagcaggtttcctcattcctgaaagaaacagttaactttcagaagagatgggcttgttttcttgccaatgaggtctgaaatggaggtccttctgctggataaaatgaggttcaactgttgattgcaggaataaggccttaatatgttaacctcagtgtcatttatgaaaagaggggaccagaagccaaagacttagtatattttcttttcctctgtcccttcccccataagcctccatttagttctttgttatttttgtttcttccaaagcacattgaaagagaaccagtttcaggtgtttagttgcagactcagtttgtcagactttaaagaataatatgctgccaaattttggccaaagtgttaatcttaggggagagctttctgtccttttggcactgagatatttattgtttatttatcagtgacagagttcactataaatggtgtttttttaatagaatataattatcggaagcagtgccttccataattatgacagttatactgtcggttttttttaaataaaagcagcatctgctaataaaacccaacagatactggaagttttgcatttatggtcaacacttaagggttttagaaaacagccgtcagccaaatgtaattgaataaagttgaagctaagatttagagatgaattaaatttaattaggggttgctaagaagcgagcactgaccagataagaatgctggttttcctaaatgcagtgaattgtgaccaagttataaatcaatgtcacttaaaggctgtggtagtactcctgcaaaattttatagctcagtttatccaaggtgtaactctaattcccatttgcaaaatttccagtacctttgtcacaatcctaacacattatcgggagcagtgtcttccataatgtataaagaacaaggtagtttttacctaccacagtgtctgtatcggagacagtgatctccatatgttacactaagggtgtaagtaattatcgggaacagtgtttcccataattttcttcatgcaatgacatcttcaaagcttgaagatcgttagtatctaacatgtatcccaactcctataattccctatcttttagttttagttgcagaaacattttgtggtcattaagcattgggtgggtaaattcaaccactgtaaaatgaaattactacaaaatttgaaatttagcttgggtttttgttacctttatggtttctccaggtcctctacttaatgagatagcagcatacatttataatgtttgctattgacaagtcattttaatttatcacattatttgcatgttacctcctataaacttagtgcggacaagttttaatccagaattgaccttttgacttaaagcagagggactttgtatagaaggtttgggggctgtggggaaggagagtcccctgaaggtctgacacgtctgcctacccattcgtggtgatcaattaaatgtaggtatgaataagttcgaagctccgtgagtgaaccatcatataaacgtgtagtacagctgtttgtcatagggcagttggaaacggcctcctagggaaaagttcatagggtctcttcaggttcttagtgtcacttacctagatttacagcctcacttgaatgtgtcactactcacagtctctttaatcttcagttttatctttaatctcctcttttatcttggactgacatttagcgtagctaagtgaaaaggtcatagctgagattcctggttcgggtgttacgcacacgtacttaaatgaaagcatgtggcatgttcatcgtataacacaatatgaatacagggcatgcattttgcagcagtgagtctcttcagaaaacccttttctacagttagggttgagttacttcctatcaagccagtacgtgctaacaggctcaatattcctgaatgaaatatcagactagtgacaagctcctggtcttgagatgtcttctcgttaaggagtagggccttttggaggtaaaggtata

SEQ ID NO: 42 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 43 - DNA, 4816 bp, Homo sapiens
gttcccggatttttgtgggcgcctgccccgcccctcgtccccctgctgtgtccatatatcgaggcgatagggttaagggaaggcggacgcctgatgggttaatgagcaaactgaagtgttttccatgatcttttttgagtcgcaattgaagtaccacctcccgagggtgattgcttccccatgcggggtagaacctttgctgtcctgttcaccactctacctccagcacagaatttggcttatgcctactcaatgtgaagatgatgaggatgaaaacctttgtgatgatccacttccacttaatgaatggtggcaaagcaaagctatattcaagaccacatgcaaagctactccctgagcaaagagtcacagataaaacgggggcaccagtagaatggccaggacaaacgcagtgcagcacagagactcagaccctggcagccatgcctgcgcaggcagtgatgagagtgacatgtactgttgtggacatgcacaaaagtgagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 44 - DNA, 3678 bp, Homo sapiens
ccccgcgcggcgcgggccagggaagggccacccaggggtcccccacttcccgcttgggcgcccggacggcgaatggagcaggggcgcgcagataattaaagatttacacacagctggaagaaatcatagagaagccgggcgtggtggctcatgcctataatcccagcacttttggaggctgaggcgggcagatcacttgagatcaggagttcgagaccagcctggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctctttaaaaaaaaaaa

SEQ ID NO: 45 - DNA, 4006 bp, Homo sapiens
tgccaaggctccagcccggccgggctccgaggcgagaggctgcatggagtggccggcgcggctctgcgggctgtgggcgctgctgctctgcgccggcggcgggggcgggggcgggggcgccgcgcctacggaaactcagccacctgtgacaaatttgagtgtctctgttgaaaacctctgcacagtaatatggacatggaatccacccgagggagccagctcaaattgtagtctatggtattttagtcattttggcgacaaacaagataagaaaatagctccggaaactcgtcgttcaatagaagtacccctgaatgagaggatttgtctgcaagtggggtcccagtgtagcaccaatgagagtgagaagcctagcattttggttgaaaaatgcatctcacccccagaaggtgatcctgagtctgctgtgactgagcttcaatgcatttggcacaacctgagctacatgaagtgttcttggctccctggaaggaataccagtcccgacactaactatactctctactattggcacagaagcctggaaaaaattcatcaatgtgaaaacatctttagagaaggccaatactttggttgttcctttgatctgaccaaagtgaaggattccagttttgaacaacacagtgtccaaataatggtcaaggataatgcaggaaaaattaaaccatccttcaatatagtgcctttaacttcccgtgtgaaacctgatcctccacatattaaaaacctctccttccacaatgatgacctatatgtgcaatgggagaatccacagaattttattagcagatgcctattttatgaagtagaagtcaataacagccaaactgagacacataatgttttctacgtccaagaggctaaatgtgagaatccagaatttgagagaaatgtggagaatacatcttgtttcatggtccctggtgttcttcctgatactttgaacacagtcagaataagagtcaaaacaaataagttatgctatgaggatgacaaactctggagtaattggagccaagaaatgagtataggtaagaagcgcaattccacactctacataaccatgttactcattgttccagtcatcgtcgcaggtgcaatcatagtactcctgctttacctaaaaaggctcaagattattatattccctccaattcctgatcctggcaagatttttaaagaaatgtttggagaccagaatgatgatactctgcactggaagaagtacgacatctatgagaagcaaaccaaggaggaaaccgactctgtagtgctgatagaaaacctgaagaaagcctctcagtgatggagataatttatttttaccttcactgtgaccttgagaagattcttcccattctccatttgttatctgggaacttattaaatggaaactgaaactactgcaccatttaaaaacaggcagctcataagagccacaggtctttatgttgagtcgcgcaccgaaaaactaaaaataatgggcgctttggagaagagtgtggagtcattctcattgaattataaaagccagcaggcttcaaactaggggacaaagcaaaaagtgatgatagtggtggagttaatcttatcaagagttgtgacaacttcctgagggatctatacttgctttgtgttctttgtgtcaacatgaacaaattttatttgtaggggaactcatttggggtgcaaatgctaatgtcaaacttgagtcacaaagaacatgtagaaaacaaaatggataaaatctgatatgtattgtttgggatcctattgaaccatgtttgtggctattaaaactcttttaacagtctgggctgggtccggtggctcacgcctgtaatcccagcaatttgggagtccgaggcgggcggatcactcgaggtcaggagttccagaccagcctgaccaaaatggtgaaacctcctctctactaaaactacaaaaattaactgggtgtggtggcgcgtgcctgtaatcccagctactcgggaagctgaggcaggtgaattgtttgaacctgggaggtggaggttgcagtgagcagagatcacaccactgcactctagcctgggtgacagagcaagactctgtctaaaaaacaaaacaaaacaaaacaaaacaaaaaaacctcttaatattctggagtcatcattcccttcgacagcattttcctctgctttgaaagccccagaaatcagtgttggccatgatgacaactacagaaaaaccagaggcagcttctttgccaagacctttcaaagccattttaggctgttaggggcagtggaggtagaatgactccttgggtattagagtttcaaccatgaagtctctaacaatgtattttcttcacctctgctactcaagtagcatttactgtgtctttggtttgtgctaggcccccgggtgtgaagcacagaccccttccaggggtttacagtctatttgagactcctcagttcttgccacttttttttttaatctccaccagtcatttttcagaccttttaactcctcaattccaacactgatttccccttttgcattctccctccttcccttccttgtagccttttgactttcattggaaattaggatgtaaatctgctcaggagacctggaggagcagaggataattagcatctcaggttaagtgtgagtaatctgagaaacaatgactaattcttgcatattttgtaacttccatgtgagggttttcagcattgatatttgtgcattttctaaacagagatgaggtggtatcttcacgtagaacattggtattcgcttgagaaaaaaagaatagttgaacctatttctctttctttacaagatgggtccaggattcctcttttctctgccataaatgattaattaaatagcttttgtgtcttacattggtagccagccagccaaggctctgtttatgcttttggggggcatatattgggttccattctcacctatccacacaacatatccgtatatatcccctctactcttacttcccccaaatttaaagaagtatgggaaatgagaggcatttcccccaccccatttctctcctcacacacagactcatattactggtaggaacttgagaactttatttccaagttgttcaaacatttaccaatcatattaatacaatgatgctatttgcaattcctgctcctaggggaggggagataagaaaccctcactctctacaggtttgggtacaagtggcaacctgcttccatggccgtgtagaagcatggtgccctggcttctctgaggaagctggggttcatgacaatggcagatgtaaagttattcttgaagtcagattgaggctgggagacagccgtagtagatgttctactttgttctgctgttctctagaaagaatatttggttttcctgtataggaatgagattaattcctttccaggtattttataattctgggaagcaaaacccatgcctccccctagccatttttactgttatcctatttagatggccatgaagaggatgctgtgaaattcccaacaaacattgatgctgacagtcatgcagtctgggagtggggaagtgatcttttgttcccatcctcttcttttagcagtaaaatagctgagggaaaagggagggaaaaggaagttatgggaatacctgtggtggttgtgatccctaggtcttgggagctcttggaggtgtctgtatcagtggatttcccatcccctgtgggaaattagtaggctcatttactgttttaggtctagcctatgtggattttttcctaacatacctaagcaaacccagtgtcaggatggtaattcttattctttcgttcagttaagtttttcccttcatctgggcactgaagggatatgtgaaacaatgttaacatttttggtagtcttcaaccagggattgtttctgtttaacttcttataggaaagcttgagtaaaataaatattgtctttttgtatgtca

SEQ ID NO: 46 - DNA, 2154 bp, Homo sapiens
cgccacgcactcctctttctgcctggccggccactcccgtctgctgtgacgcgcggacagagagctaccggtggacccacggtgcctccctccctgggatctacacagaccatggccttgccaacggctcgacccctgttggggtcctgtgggacccccgccctcggcagcctcctgttcctgctcttcagcctcggatgggtgcagccctcgaggaccctggctggagagacagggcaggaggctgcgcccctggacggagtcctggccaacccacctaacatttccagcctctcccctcgccaactccttggcttcccgtgtgcggaggtgtccggcctgagcacggagcgtgtccgggagctggctgtggccttggcacagaagaatgtcaagctctcaacagagcagctgcgctgtctggctcaccggctctctgagccccccgaggacctggacgccctcccattggacctgctgctattcctcaacccagatgcgttctcggggccccaggcctgcacccgtttcttctcccgcatcacgaaggccaatgtggacctgctcccgaggggggctcccgagcgacagcggctgctgcctgcggctctggcctgctggggtgtgcgggggtctctgctgagcgaggctgatgtgcgggctctgggaggcctggcttgcgacctgcctgggcgctttgtggccgagtcggccgaagtgctgctaccccggctggtgagctgcccgggacccctggaccaggaccagcaggaggcagccagggcggctctgcagggcgggggacccccctacggccccccgtcgacatggtctgtctccacgatggacgctctgcggggcctgctgcccgtgctgggccagcccatcatccgcagcatcccgcagggcatcgtggccgcgtggcggcaacgctcctctcgggacccatcctggcggcagcctgaacggaccatcctccggccgcggttccggcgggaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggctcctcggcggcccctcccacaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcatgcaagaggccctctcggggacgccctgcctcctaggacctggacctgttctcaccgtcctggcactgctcctagcctccaccctggcctgagggccccactcccttgctggccccagccctgctggggatccccgcctggccaggagcaggcacgggtggtccccgttccaccccaagagaactcgcgctcagtaaacgggaacatgccccctgcagacacgtaaaaaaaaaaaaaaaaaa

SEQ ID NO: 47 - DNA, 469 bp, Homo sapiens
agccctccaggacaggctgcatcagaagaggccatcaagcagatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgcaggcagccccacacccgccgcctcctgcaccgagagagatggaataaagcccttgaaccagcaaaa

SEQ ID NO: 48 - DNA, 1376 bp, Homo sapiens
gtaagaacactctcgtgagtctaacggtcttccggatgaaggctatttgaagtcgccataacctggtcagaagtgtgcctgtcggcggggagagaggcaatatcaaggttttaaatctcggagaaatggctttcgtttgcttggctatcggatgcttatatacctttctgataagcacaacatttggctgtacttcatcttcagacaccgagataaaagttaaccctcctcaggattttgagatagtggatcccggatacttaggttatctctatttgcaatggcaacccccactgtctctggatcattttaaggaatgcacagtggaatatgaactaaaataccgaaacattggtagtgaaacatggaagaccatcattactaagaatctacattacaaagatgggtttgatcttaacaagggcattgaagcgaagatacacacgcttttaccatggcaatgcacaaatggatcagaagttcaaagttcctgggcagaaactacttattggatatcaccacaaggaattccagaaactaaagttcaggatatggattgcgtatattacaattggcaatatttactctgttcttggaaacctggcataggtgtacttcttgataccaattacaacttgttttactggtatgagggcttggatcatgcattacagtgtgttgattacatcaaggctgatggacaaaatataggatgcagatttccctatttggaggcatcagactataaagatttctatatttgtgttaatggatcatcagagaacaagcctatcagatccagttatttcacttttcagcttcaaaatatagttaaacctttgccgccagtctatcttacttttactcgggagagttcatgtgaaattaagctgaaatggagcatacctttgggacctattccagcaaggtgttttgattatgaaattgagatcagagaagatgatactaccttggtgactgctacagttgaaaatgaaacatacaccttgaaaacaacaaatgaaacccgacaattatgctttgtagtaagaagcaaagtgaatatttattgctcagatgacggaatttggagtgagtggagtgataaacaatgctgggaaggtgaagacctatcgaagaaaactttgctacgtttctggctaccatttggtttcatcttaatattagttatatttgtaaccggtctgcttttgcgtaagccaaacacctacccaaaaatgattccagaatttttctgtgatacatgaagactttccatatcaagagacatggtattgactcaacagtttccagtcatggccaaatgttcaatatgagtctcaataaactgaatttttcttgcgaatgttgaaaaa

SEQ ID NO: 49 - DNA, 9059 bp, Homo sapiens
gagaaggacgcgcggcccccagcgcctcttgggtggccgcctcggagcatgacccccgcgggccagcgccgcgcgctctgatccgaggagaccccgcgctcccgcagccatggccaccgggggccggcggggggcggcggccgcgccgctgctggtggcggtggccgcgctgctactgggcgccgcgggccacctgtaccccggagaggtgtgtcccggcatggatatccggaacaacctcactaggttgcatgagctggagaattgctctgtcatcgaaggacacttgcagatactcttgatgttcaaaacgaggcccgaagatttccgagacctcagtttccccaaactcatcatgatcactgattacttgctgctcttccgggtctatgggctcgagagcctgaaggacctgttccccaacctcacggtcatccggggatcacgactgttctttaactacgcgctggtcatcttcgagatggttcacctcaaggaactcggcctctacaacctgatgaacatcacccggggttctgtccgcatcgagaagaacaatgagctctgttacttggccactatcgactggtcccgtatcctggattccgtggaggataattacatcgtgttgaacaaagatgacaacgaggagtgtggagacatctgtccgggtaccgcgaagggcaagaccaactgccccgccaccgtcatcaacgggcagtttgtcgaacgatgttggactcatagtcactgccagaaagtttgcccgaccatctgtaagtcacacggctgcaccgccgaaggcctctgttgccacagcgagtgcctgggcaactgttctcagcccgacgaccccaccaagtgcgtggcctgccgcaacttctacctggacggcaggtgtgtggagacctgcccgcccccgtactaccacttccaggactggcgctgtgtgaacttcagcttctgccaggacctgcaccacaaatgcaagaactcgcggaggcagggctgccaccagtacgtcattcacaacaacaagtgcatccctgagtgtccctccgggtacacgatgaattccagcaacttgctgtgcaccccatgcctgggtccctgtcccaaggtgtgccacctcctagaaggcgagaagaccatcgactcggtgacgtctgcccaggagctccgaggatgcaccgtcatcaacgggagtctgatcatcaacattcgaggaggcaacaatctggcagctgagctagaagccaacctcggcctcattgaagaaatttcagggtatctaaaaatccgccgatcctacgctctggtgtcactttccttcttccggaagttacgtctgattcgaggagagaccttggaaattgggaactactccttctatgccttggacaaccagaacctaaggcagctctgggactggagcaaacacaacctcaccatcactcaggggaaactcttcttccactataaccccaaactctgcttgtcagaaatccacaagatggaagaagtttcaggaaccaaggggcgccaggagagaaacgacattgccctgaagaccaatggggaccaggcatcctgtgaaaatgagttacttaaattttcttacattcggacatcttttgacaagatcttgctgagatgggagccgtactggccccccgacttccgagacctcttggggttcatgctgttctacaaagaggccccttatcagaatgtgacggagttcgacgggcaggatgcgtgtggttccaacagttggacggtggtagacattgacccacccctgaggtccaacgaccccaaatcacagaaccacccagggtggctgatgcggggtctcaagccctggacccagtatgccatctttgtgaagaccctggtcaccttttcggatgaacgccggacctatggggccaagagtgacatcatttatgtccagacagatgccaccaacccctctgtgcccctggatccaatctcagtgtctaactcatcatcccagattattctgaagtggaaaccaccctccgaccccaatggcaacatcacccactacctggttttctgggagaggcaggcggaagacagtgagctgttcgagctggattattgcctcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccagaaaaacctcttcaggcactggtgccgaggaccctaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagctgcaggcttgcaaccaggacacccctgaggaacggtgcagtgtggcagcctacgtcagtgcgaggaccatgcctgaagccaaggctgatgacattgttggccctgtgacgcatgaaatctttgagaacaacgtcgtccacttgatgtggcaggagccgaaggagcccaatggtctgatcgtgctgtatgaagtgagttatcggcgatatggtgatgaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactatttagacgtcccgtcaaatattgcaaaaattatcatcggccccctcatctttgtctttctcttcagtgttgtgattggaagtatttatctattcctgagaaagaggcagccagatgggccgctgggaccgctttacgcttcttcaaaccctgagtatctcagtgccagtgatgtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcacgtggtgcgcctcctgggagtggtgtccaagggccagcccacgctggtggtgatggagctgatggctcacggagacctgaagagctacctccgttctctgcggccagaggctgagaataatcctggccgccctccccctacccttcaagagatgattcagatggcggcagagattgctgacgggatggcctacctgaacgccaagaagtttgtgcatcgggacctggcagcgagaaactgcatggtcgcccatgattttactgtcaaaattggagactttggaatgaccagagacatctatgaaacggattactaccggaaagggggcaagggtctgctccctgtacggtggatggcaccggagtccctgaaggatggggtcttcaccacttcttctgacatgtggtcctttggcgtggtcctttgggaaatcaccagcttggcagaacagccttaccaaggcctgtctaatgaacaggtgttgaaatttgtcatggatggagggtatctggatcaacccgacaactgtccagagagagtcactgacctcatgcgcatgtgctggcaattcaaccccaagatgaggccaaccttcctggagattgtcaacctgctcaaggacgacctgcaccccagctttccagaggtgtcgttcttccacagcgaggagaacaaggctcccgagagtgaggagctggagatggagtttgaggacatggagaatgtgcccctggaccgttcctcgcactgtcagagggaggaggcggggggccgggatggagggtcctcgctgggtttcaagcggagctacgaggaacacatcccttacacacacatgaacggaggcaagaaaaacgggcggattctgaccttgcctcggtccaatccttcctaacagtgcctaccgtggcgggggcgggcaggggttcccattttcgctttcctctggtttgaaagcctctggaaaactcaggattctcacgactctaccatgtccaatggagttcagagatcgttcctatacatttctgttcatcttaaggtggactcgtttggttaccaatttaactagtcctgcagaggatttaactgtgaacctggagggcaaggggtttccacagttgctgctcctttggggcaacgacggtttcaaaccaggattttgtgttttttcgttccccccacccgcccccagcagatggaaagaaagcacctgtttttacaaattctttttttttttttttttttttgctggtgtctgagcttcagtataaaagacaaaacttcctgtttgtggaacaaaagttcgaaagaaaaaacaaaacaaaaacacccagccctgttccaggagaatttcaagttttacaggttgagcttcaagatggtttttttggttttttttttttctctcatccaggctgaaggattttttttttctttacaaaatgagttcctcaaattgaccaatagctgctgctttcatattttggataagggtctgtggtcccggcgtgtgctcacgtgtgtatgcacgtgtgtgtgtccattagacacggctgatgtgtgtgcaaagtatccatgcggagttgatgctttgggaattggctcatgaaggttcttctcaagggtgcgagctcatccccctctctccttccttcttattgactgggagactgtgctctcgacagattcttcttgtgtcagaagtctagcctcaggtttctaccctcccttcacattggtggccaagggaggagcatttcatttggagtgattatgaatcttttcaagaccaaaccaagctaggacattaaaaaaaaaaaaagaaaaagaaagaaaaaacaaaatggaaaaaggaaaaaaaaaaagaactgagatgacagagttttgagaatatatttgtaccatatttaatttttaaagtctctggtattagcctcataagttattgactattccccggggttggcggggagtggggacatgagttggtctgcctgttgtggggccgggaaggggagggagtcaggcacaagtggcctctttgtttggtcttaaaggcatccatttctgggaatgaagccatgttcgctgctaacacttttggatgttgtgaggccacgtggagtgtgtgagagactaggttttatggatggtctggttcaggtaccaggtctgctggaaggttcctgttcggataagctggtagctacctagctctgagcctgccttcaagaacacctgtgttcatcctctgattctctgtgtgtacctcttgtggcgtttcctctcccgggtgtgaacatcctaaccgttattgtgcaaacccaagaacgtcagatcccaaagcacaacaacctggatggactttgggaacatctaagcaatgtaagagagaggtgcactgagagtacgtcttggtcccctccaccctgagagcatctgacggtcctcagtactgaactcccggaagctgctctgagcccggtgacctcatctgggccaggtgtggtgcctgagctgaatgctcaggtgcttacagtgttgcaatccctaagagagtagagtctggaggagaaaccgtgaaaaagaccttacacaccaccaagaacttccgaatgggcgtgaatccaccgtttcttctctttgcaaaaagaaccaccacagctgctcaaagaacacagtgaactcatcactttggttcatcaaaaaatcatcgcccatgcgttattcctgagtgcattttcttacaactttttgactgcttccttttcttcttctcttaagagttgtgggcttaagaatgggatagagtcataatggcaacctccaagccctctcaattcttgattaagaacacaggtagacatgaatcccaattgtctattgctatcttatttatatgattcgggaaaatacagcatgtaaaaatattgctgaggagcctcagtgattgggtacaagaagcaagagtacagaaattatttttgccaaatttattttgtaaatatgagggtctgtacctaaatttaaaaaaaaaacacgtagaactaggtattttgttctcttcttagtaaatttgtagtggttgtatactacactagctgcaattttcacatttttctaattcagaaaggtttttcttatattaggggaaaaagtatttattttaatatataaaatcactctgaaaatcactctcataaaaaatggagcgcatgtaaatttttatcaaagaaaaataaacaggtgaatgggggatagtgattttcttttttcagcacagtctacctcagtgtattgttaagatgtgattcaatcatggacatctttgagatttcagaattctacctggaaccggtctgaatcagggaacgtgtgtatcagctgattcgaatgccagggaccagtaagaattttgagggagggagttgggatggagaaggtatggcctttatgcgagcatagatccttttcttcctggctggtaatattcttctctgaatttaatcttcctttaaaaaaaaatcctccatctattgtcactatgttccccaaacataaactaagttccaggctgtcatgatgtatctgatatatggggtaacccagcaaggtgtaccttcctttggtgagagatggctgccggggcaaagacgggctttgattcagagcaagcattcccacctgttccatggaatccccctgaagtgagcacaaaggtgccctgggctccctgatggtttatgcccactcctttcaggctggtgatgcaccttacacacaaacacctaatgcaatgtctttttaaattctccaagtgggatgggagcatgtgagggaaattccaatccaaaacccattaatgtgctgaacgctttttttttttttttttttttttttgcaacaacaccttggacctctgtgttggggtttgactgacctcaagctgatattattggaccttgtgcagctttgataacccatgtgagagtctaggcaggaccagtggggcccaaatcttgctgctcttgtacttttaggcactgcccttgcagactcacctttctccacctgccctggagaaaggtagggtgtgctgggcctgccccttgcaaatgggattcaccagtttcatttatttgactctactgccacagtgaaaagagcaaacagctattgggttgcaaacctcctttgacattaggaaatgttgactttgtaacaataaaactttggtcctagaaagacacggttgtcctgggagtttgtagtgttaagttgcaacaacaacaacaaaaagcaacaaaaccagcttaggataacactttttgttgcttgttcttaaagatgtctcactatgattaaaacccttttcattaatgtagtgaaagccacacaggagttccttcttccaggaggagaataccaagcacatcactttctctctgcatcagtgatgtcaaatacgcatcagaaaatgttcaggttttaggagctgtcctaggtgctgtttcatcattggaagcagtgagaaagagaagcactgctgcttgtctggatataggctgaggatgattgagagaagctgtgggaactgacacaagggtctgcataggtcatcctgtgaccctggggactatgttaccaactgacagacagatctttcactgtatcctagcagggcaggtagtccaccaagaaatgtgcttattggattgggaggtgtttatttgtagtctgctgtaacacgtgtgaaagagcaggagcgtcatcagcatatgacttgcgctggtcatccggtaaatggatgtgctgtagtcccagtgctaatcatttctctccttcacagtgggtggaagtttagggttaaatgtcctttgaatgtcacctggtgagtccttgacaccttaggctcttcagaaacaatggttttgttgaggatggggaacagggaatgccgattttatatacatggtacacagagaggggtgtcacttcagaaaatcttccagcatgttcttcagaatattaatttatatgcgaggtgaggttgggaatgaaaagaacaggtcagcacttttttttttcctagaacatacaaaagaacatggtggactttcagggagtgcaatggaaggtgaatatttccttaagggtccccgagaaatgggagtgaggggaggggacacaatggctttttgagcttacttttaccttctgatactagtcaaggtccagaaccagccaccagccaaatttctatctgggtgcgggccactgaaaatccttgttaaaaaccagatcacaaatctggggctcttggtcccattggagaaggaaggaagagcctcaaaataagtgtgcacccatgcacatattcaggaacagcttgtttagtctttacactttgcctgaaagttgcttctcctcgtccctttgtgtgcctgggtggcctcggccctgtgcgttggcaacgcaggatcaaatgtgctgcagcttttgcagaaaacaactcagaaacacaaaaccccccaacagctcaattattattttttcaatgttttcctacaagagccaagtagcaccatgtacagaagacgcctttttttttggaatattgaaatcgttctgcatgtaaaatatgggataatgacctgtttatattaaaattctgattaaattatctgagaata

(19) United States
(12) Patent Application Publication
(10) Pub. No.: US 2010/0284921 A1
(43) Pub. Date: Nov. 11, 2010

(54) TARGETED NANOPARTICLES FOR INTRACELLULAR CANCER THERAPY

(75) Inventors: JENNIFER GORDON, PHILADELPHIA, PA (US); LINDA KNIGHT, BRYN MAWR, PA (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: TEMPLE UNIVERSITY - OF THE COMMONWEALTH SYSTEM OF HIGHER EDUCATION, PHILADELPHIA, PA (US)

(21) Appl. No.: 12/776,503

(22) Filed: May 10, 2010

Related U.S. Application Data
(60) Provisional application No. 61/215,717, filed on May 8, 2009.

(51) Int. Cl. A61K 49/00; G01N 33/574; A61P 35/00

(52) U.S. Cl. 424/9.1; 435/7.23

(57) ABSTRACT

This invention provides constructs comprising a targeting member immobilized on a detectable particulate, in which binding of the targeting member to a target structure on a surface of a cancer cell triggers internalization of the construct. Such constructs can be used to identify or monitor cancer cells in cell cultures or in a tissue. Such construct can also be used to kill or prevent growth of cancer cells in vivo. Also included in the invention are methods for killing or preventing growth of cancer cells in vivo.

### CROSS-REFERENCE TO RELATED APPLICATIONS

This application claims the benefit of U.S. Provisional Application No. 61/215,717, filed May 8, 2009, the entire disclosure of which is incorporated herein by reference.

### FIELD OF INVENTION

The invention relates to a construct of a targeting member immobilized on a detectable particulate, wherein the targeting member binds selectively to a target structure that is preferentially expressed on the surface of cancer cells. Upon binding of the immobilized targeting member to the target structure, the construct undergoes internalization by the cancer cell. The invention also relates to the in vitro and in vivo imaging of cancer cells using such construct. The invention further relates to the treatment of cancer in a mammal using such construct.

### BACKGROUND OF INVENTION

Cancer is characterized by a cell mass with uncontrolled cellular division and unstable chromosomal material. Cancer cells grow with minimal or impaired control, and have the ability to invade and/or destroy adjacent tissue. Sometimes cancer cells may metastasize to other locations in the body, spreading via lymph or blood vessels. These properties distinguish cancerous tumors from so-called “benign tumors”, which are self-limited, do not invade or metastasize. Even though most cancers are in solid form, there are well-known non-solid tumors, such as leukemia. Cancer treatments are far from effective or convenient, and are generally restricted to chemotherapy, surgery or radiation, which may harm both surrounding and distal normal tissue. Greater understanding of the biology of cancer may help identify novel and efficient treatment options.

Abnormalities in the genetic material of the cancer cells may be inherited or may be caused by contact with carcinogens (such as tobacco smoke, radiation, and chemicals) or infection by microorganisms (such as oncoviruses). Genetic abnormalities found in cancer typically cause activation (or upregulation) of oncogenes and/or inactivation (or downregulation) of tumor suppressor genes. Activation of cancer-promoting oncogenes in cancer cells lead to nefarious effects, such as hyperactive growth and division, avoidance of programmed cell death, penetration of normal tissue boundaries, and ability to become established in diverse tissue environments. Inactivation of tumor suppressor genes in cancer cells results in the loss of growth-controlling functions. This may impair accurate DNA replication, control over the cell cycle, orientation and adhesion within tissues, and interaction with protective cells of the immune system.

Oncoviruses are among the many cancer-causing organisms identified so far. Oncoviruses may have a DNA genome, such as adenovirus and human polyomavirus (JC virus), or an RNA genome, such as the human T-cell leukemia virus. Polyomaviruses have small circular genomes encoding only six proteins including three structural capsid proteins (Frisque and White, 1993, “The molecular biology of JCV, causative agent of progressive multifocal leukoencephalopathy”, Molecular Neurovirology, pp 25-128, R. P. Roos, Ed., Humana Press, Totowa, N.J.). In 1971, the JC virus (JCV) was isolated from the brain of a patient suffering from the demyelinating disease PML (progressive multifocal leukoencephalopathy). More than 80% of the population becomes infected with the human polyomavirus JC virus during childhood, but in most infected individuals the virus remains latent in the kidney without causing any overt signs of disease. However, in immunocompromised individuals, such as AIDS patients, transplant recipients and individuals with lymphoproliferative disorders, reactivation of JCV results in the fatal PML (Berger and Concha, 1995, J. Neurovirol. 1:5-18). Over the last several years, studies have suggested a role for JCV in human cancer as a broad range of CNS tumors (medulloblastoma, glioblastoma, astrocytoma, oligodendroglioma and other tumors of neural crest origin). Furthermore, colorectal carcinoma has been found to harbor JCV DNA sequences and to express the viral regulatory protein T-antigen (Del Valle et al., 2001, In: “Human polyomaviruses: Molecular and clinical perspectives”, pp 409-430, K. Khalili and G. L. Stoner, Eds., Wiley Liss, New York, N.Y.). In vitro studies suggest that T-antigen may target the Wnt signaling pathway via 13-catenin in colon cancer and medulloblastoma (Enam et al., 2002, Cancer Res. 2002:7093-7101; Gan et al., 2001, Oncogene 20:4864-4870). The T-antigen plays a critical role in the viral life cycle since it directs viral early and late gene expression and also viral DNA replication during lytic infection (Frisque and White, 1993, “The molecular biology of JCV, causative agent of progressive multifocal leukoencephalopathy”, Molecular Neurovirology, pp 25-128, R. P. Roos, Ed., Humana Press, Totowa, N.J.). In addition to its role in viral regulation during active replication, JCV T-antigen is considered an oncogene due to its demonstrated ability to transform cells in culture. Cells expressing JCV T-antigen exhibit characteristics of transformed or immortalized cells, including morphological changes such as multinucleation, rapid doubling time, growth in anchorage independence, and subcutaneous growth in the nude mouse. In addition, JCV T-antigen has been shown to sequester and inactivate the tumor suppressor protein p53 and the retinoblastoma protein family members, pRb, p130, and p107.

Another oncovirus of interest is human T-cell leukemia virus-1. This virus causes the expression of the HTLV Tax protein on the surface of cancer cells. The Tax protein is crucial for viral replication and for initiating malignant transformation leading to the development of adult T-cell leukemia. Tax has been shown to be oncogenic, since it transforms and immortalizes rodent fibroblasts and human T-lymphocytes. Through CREB, NF-B and SRF pathways, Tax transactivates cellular promoters including those of cytokines (IL-13, IL-15), cytokine receptors (IL-2R) and costimulatory surface receptors (OX40/OX40L), leading to upregulated protein expression and activated signaling cascades (e.g. Jak/STAT, PI3Kinase, JNK). Tax also stimulates cell growth by direct binding to cyclin-dependent kinase holenzymes and/or inactivating tumor suppressors (e.g. p53, DLG). Moreover, Tax silences cellular checkpoints, which guard against DNA structural damage and chromosomal missegregation, thereby favoring the manifestation of a mutator phenotype in cells (Lin et al., 2005, “Activation of human T cell leukemia virus type 1 LTR promoter and cellular promoter elements by T cell receptor signaling and HTLV-a Tax expression”, Virology 339:1-11; Nicot et al., 1998, “Cytoplasmic forms of human T-cell leukemia virus type 1 Tax induce NF-kB activation”, J. Virol. 72:6777-6784).

Other known examples of DNA oncoviruses are: Hepatitis B & C viruses, which may cause liver cancer; human papilloma virus (HPV), which causes transformation in cells through interfering with tumor suppressor proteins such as p53; human herpes virus 8, associated with Kaposi's sarcoma, a type of skin cancer (Chang et al., 1994, Science 266:1865-1869); and Epstein Barr Virus (EBV), associated with four types of cancers (Burkitt's lymphoma, Hodgkin's lymphoma, B-lymphoproliferative disease and nasopharyngeal carcinoma). Examples of known RNA oncoviruses are hepatitis C virus and human T-cell leukemia virus-1 (HTLV-1). An example of a known oncobacterium is H. pylori (gastric cancer).

In the process of studying cancer cells and their altered metabolism, scientists have been able to identify receptors or proteins that are characteristically expressed in cancer cells. Some of these receptors or proteins may be generally associated with genetic abnormalities, where oncogenes are activated and/or tumor suppressor genes are inactivated, or may be associated with cell types that multiply out of control as part of a cancer event. Other receptors or proteins may be expressed in normal cells at lower levels, but are characteristically overexpressed or activated in cancer cells, acting as a cancer marker.

Several receptors or proteins characteristically expressed in cancer cells are displayed on the outer surface of the cell membrane. By being present on the outer surface of the cell membrane, these receptors or proteins are able to interact with external biological molecules and act as cancer markers. The presence of these receptors or protein on the cell surface also facilitates the identification of cells that are actively expressing them. Non-limiting examples of such receptors and proteins are CD7, CD19, CD22, CD25 (or corresponding IL-2R), CD30, CD33, CD56, Le^{y}, TFR, EGFR, ErbB2, IL-4R, IL-13R, and mesothelin. For general review of these receptors and proteins and their use in targeting cancer cells, see Kreitman, 2006, The AAPS J. 8 (3):E532-E551, incorporated herein in its entirety for reference.

CD7 (Cluster of Differentiation 7), a transmembrane protein, is a member of the immunoglobulin superfamily found on thymocytes and mature T cells (Stillwell and Bierer, 2002, Immunol. Res. 24 (1):31-52; Aruffo & Seed, 1987, EMBO J. 6:3313-3316). CD7 plays an essential role in T-cell interactions and also in T-cell/B-cell interaction during early lymphoid development.

CD19 (Cluster of Differentiation 19) is a protein expressed on follicular dendritic cells and B cells, from earliest recognizable B-lineage cells to B-cell blasts but is lost on maturation to plasma cells (Ishikawa et al., 2003, Leuk. Lymphoma 43 (3):613-6; Tedder & Isaacs, 1989, J. Immun. 143:712-717). This cell surface molecule assembles with the antigen receptors of B-lymphocytes (CD21 and CD81) in order to decrease the threshold for antigen receptor-dependent stimulation. Upon activation, CD19 binds to Src-family kinases and recruits PI-3 kinase.

CD22 (Cluster of Differentiation 22) is a sugar-binding transmembrane protein, which specifically binds sialic acid and has an immunoglobulin (Ig) domain located at its N-terminus (Crocker et al., 1998, Glycobiology 8 (2):v-vi; Wilson et al., 1991, J. Exp. Med. 173:137-146). CD22 functions as an inhibitory receptor for B-cell receptor (BCR) signalling. EBV (Epstein-Barr virus) is known to bind to the B-lymphocyte via the CD22 receptor.

CD25 is the α-chain of the interleukin-2 receptor (IL-2 receptor or IL-2R), a heterotrimeric protein expressed on the surface of certain immune cells, such as lymphocytes (Smith, 1989, Ann. Rev. Cell Biol. 5:397-425; Leonard et al., 1984, Nature 311:626-631; Nikaido et al., 1984, Nature 311:631-635; Cosman et al., 1984, Nature 312:768-771). CD25 binds and responds to a cytokine called interleukin 2. Three protein chains (α, β and γ) are non-covalently associated to form the IL-2R. The α- and β-chains are involved in binding IL-2, while signal transduction following cytokine interaction is carried out by the γ-chain, along with the β-subunit.

CD30 (also known as TNFRSF8) is a cell membrane protein of the tumor necrosis factor receptor family and a tumor marker (Durkop et al., 1992, Cell 68:421-427). This receptor is expressed by activated, but not resting, T- and B-cells, mediating the signal transduction that leads to the activation of NF-kappaB. It is a positive regulator of apoptosis, and also has been shown to limit the proliferative potential of auto-reactive CD8 effector T cells and protect the body against auto-immunity. CD30 is associated with anaplastic large cell lymphoma and expressed in embryonal carcinoma, but not in seminoma, being thus a useful marker in distinguishing between these germ cell tumors (Teng et al. 2005, Zhonghua Bing Li Xue Za Zhi 34 (11):711-715).

CD33 is a transmembrane receptor expressed on cells of monocytic/myeloid lineage and binds sialic acid (Vitale et al., 2001, Proc. Natl. Acad. Sci. U.S.A. 98:5764-5769; Peiper et al., 1988, Blood 72:314-321). The extracellular portion of this receptor contains two immunoglobulin domains (one IgV and one IgC2 domain). The intracellular portion of CD33 contains immunoreceptor tyrosine-based inhibitory motifs implicated in inhibition of cellular activity. CD33 is the target of gemtuzumab ozogamicin (Mylotarg™), a monoclonal antibody-based treated for acute myeloid leukemia (Bross et al., 2001, Clin. Cancer Res. 7 (6):1490-6).

CD56 (neural cell adhesion molecule, NCAM) is a homophilic binding glycoprotein expressed on the surface of neurons, glia, skeletal muscle and natural killer cells (Cunningham et al., 1987, Science 236:799-806). NCAM has been implicated in cell-cell adhesion, neurite outgrowth, synaptic plasticity, and learning and memory. In anatomic pathology, pathologists make use of CD56 immunohistochemistry to recognize certain tumors. Normal cells that stain positively for CD56 include NK cells, activated T cells, the brain and cerebellum, and neuroendocrine tissues. Tumors that are CD56-positive are myeloma, myeloid leukemia, neuroendocrine tumors, Wilms' tumor, adult neuroblastoma, NK/T cell lymphomas, pancreatic acinar cell carcinoma, pheochromocytoma, and small cell lung carcinoma (Ewing's sarcoma is CD56-negative). NCAM has been used as a target molecule for experimental antibody-based immunotherapy to treat neuroblastoma and cell lung cancer (Jensen and Berthold, 2007, Cancer Lett. 258 (1):9-21)

Le^{y} is a difucosylated oligosaccharide that belongs to the A-, B-, and H-Lewis blood group family. The Le^{y} antigen is expressed predominately during embryogenesis, and in adults expression is restricted to granulocytes and epithelial surfaces (Dettke et al., 2000, J. Leukoc. Biol. 68:511-514). Overexpression of Le^{y} has been shown in the majority of cancer cells derived from epithelial tissues, including breast, ovary, pancreas, prostate, esophageal, stomach, colon and non-small cell lung cancers (Hellstrom et al., 1990, Cancer Res. 50:2183-2190), either at the plasma membrane as a glycolipid or linked to surface receptors (e.g. of the ErbB family) (Basu et al., 1987, Cancer Res. 47:2531-2536).

TFR (transferrin receptor) is a carrier protein for transferrin and plays a key role in homeostasis (Schneider et al., 1984, Nature 311:675-678). TFR is needed for the import of iron into the cell and is regulated in response to intracellular iron concentration. Its overexpression has been reported in different types of cancers such as glioma, pancreatic, and colon cancers (Ryschich et al., 2004, Eur. J. Cancer 40:1418-1422; Szekeres et al., 2002, Curr. Med. Chem. 9:759-764).

EGFR (epidermal growth factor receptor, also known as ErbB1 or HER1) is the cell-surface receptor for members of the epidermal growth factor (EGF) family of extracellular protein ligands (Herbst, 2004, Int. J. Radiat. Oncol. Biol. Phys. 59 (2 Suppl):21-6; Reiter et al. 2001, Genomics 71:1-20). Mutations affecting EGFR expression or activity could result in cancer, including lung cancer and glioblastoma multiforme (Zhang et al., 2007, J. Clin. Invest. 117 (8):2051-8). Mutations involving EGFR could lead to its constant activation with uncontrolled cell division, a predisposition for cancer (Lynch et al., 2004, N. Engl. J. Med. 350 (21):2129-39). Consequently, mutations of EGFR have been identified in several types of cancer, and the receptor is the target of an expanding class of anticancer therapies. The identification of EGFR as an oncogene has led to the development of anticancer therapeutics directed against EGFR, including monoclonal antibodies cetuximab (Erbitux™, BMS), panitumumab (Vectibix™, Amgen), zalutumumab, nimotuzumab (BioMab^{EGFR}™, CIM Cuba), and matuzumab (Merck Serono & Takeda).

ErbB2 (also known as HER2/neu, CD340, ERBB2 or human epidermal growth factor receptor 2) is a protein associated with higher aggressiveness in breast cancers (Olayioye, 2001, Breast Cancer Res. 3 (6):385-389; Ullrich et al., 1984, Nature 309:418-425), and is a target of treatment in breast cancer. Approximately 15-20% of breast cancers have an amplification of the HER2/neu gene or overexpression of its protein product, with increased disease recurrence and worse prognosis. Breast tumors are thus routinely checked for overexpression of HER2/neu. Overexpression also occurs in other cancers, such as ovarian cancer and stomach cancer. Clinically, HER2/neu is important as the target of the monoclonal antibody trastuzumab (marketed as Herceptin™). Trastuzumab is only effective in breast cancer where the HER2/neu receptor is overexpressed. Another monoclonal antibody, Pertuzumab (Omnitarg™, Genentech, Le et al., 2005, Cell Cycle 4 (1):87-95), which inhibits dimerization of HER2 and HER3 receptors, is in advanced clinical trials.

IL-4R (interleukin 4 receptor) is a type I cytokine receptor that can bind interleukin 4 and interleukin 13 to regulate IgE antibody production in B-cells (Nelms et al., 1999, Annu Rev. Immunol. 17:701-738; Jiang et al., 2000, J. Allergy Clin. Immunol. 105 (6 Pt 1):1063-1070; Idzerda et al., 1990, J. Exp. Med. 171:861-873). A soluble form of this receptor can inhibit IL-4-mediated cell proliferation and IL-5 upregulation by T-cells.

IL-13R (interleukin 13 receptor) is a receptor that can bind interleukin 13, and has been shown to be an attractive target for glioma molecular therapies (Hu et al., 2005, Cancer Ther. 3:531-542; Murata et al., 1998, Int. J. Mol. Med. 1 (3):551-557; Chomarat and Banchereau, 1998, Int. Rev. Immunol. 17 (1-4):1-52). IL-13R comprises the alpha-1 chain (Aman et al., 1996, J. Biol. Chem. 271:29265-29270) and the alpha-2 chain (Caput et al., 1996, J. Biol. Chem. 271:16921-16926).

Mesothelin is a protein present on normal mesothelial cells and overexpressed in several human tumors, including mesothelioma and ovarian and pancreatic adenocarcinoma (Kojima et al., 1995, J. Biol. Chem. 270:21984-21990). The mesothelin gene encodes a precursor protein that is processed to yield mesothelin, which is attached to the cell membrane by a glycosylphosphatidyl inositol linkage and a 31-kDa shed fragment named megakaryocyte-potentiating factor (MPF). Although it has been proposed that mesothelin is related to cell adhesion, its biological function is not known. Mesothelin has been proposed as a therapeutic target for cancer treatment (Hassan et al., 2004, Clin. Cancer Res. 10:3937-3942).

Faced with the challenge of understanding the molecular source of cancer and controlling its formation and growth, scientists have long tried to develop techniques to monitor cancer cells. Such techniques should allow not only the identification but also the imaging of cancer cells in both cell cultures and in vivo. Efficient monitoring of cancer cells would help study the morphology and metabolism of a cancer cell and understand how cancer cells manage to overcome the body's defense mechanisms.

Along with the monitoring of cancer cells, cancer researchers are interested in the development of targeted therapeutics for cancer. Such therapeutics would be able to home in to cancer cells and kill them selectively, without causing toxicity or death in normal cells. The source of such selectivity for cancer cells would presumably be connected to morphological differences between cancer and normal cells, especially in terms of proteins or receptors that are selectively displayed on the outside membrane of cancer cells. Once the therapeutic agent binds to the protein or receptor displayed on the outside of the cancer cell, it would cause irreversible damage to the cancer cell membrane or enter the cancer cell itself (“internalization”) and interfere with its life cycle. A systemic treatment that does not harm normal tissue and specifically targets cancer cells could be used to treat tumors and tumor metastases at the same time.

In summary, there is a need for improved methods of monitoring and treatment of cancer. Availability of a targeted monitoring agent would allow evaluation of cancer cells in vitro and in vivo, and could allow design of patient-specific therapy and early assessing of the treatment effectiveness. At the same time, killing cancer cells, while sparing normal healthy cells, is a long-sought goal that can only be achieved by exploring the unique characteristics of cancer cells. The present invention addresses both needs.

### SUMMARY OF INVENTION

As described herein, the inventors have surprisingly discovered that a construct of a targeting member immobilized on a detectable particulate, wherein the targeting member binds selectively to a target structure that is preferentially expressed on the surface of a cancer cell, undergoes internalization by the cancer cell. The immobilized targeting member binds to the target structure, triggering internalization of the construct. Such constructs find use in the monitoring and detection of cancer cells in cell culture and in vivo, as well as in the treatment of cancerous tumors in vivo.

The invention provides compositions for the detection, monitoring or killing of cancer cells. According to an embodiment of the invention, the composition of the invention comprises a targeting member immobilized on a detectable particulate, wherein the targeting member binds selectively to a target structure that is preferentially expressed on the surface of a cancer cell and undergoes internalization by the cancer cell. In another embodiment, the composition of the invention further comprises a blood brain barrier (BBB) penetration element immobilized on the detectable particulate. In another embodiment, the composition of the invention further comprises a therapeutic agent immobilized on the detectable particulate.

According to one embodiment of the invention, the detectable particulate ranges in size from about 1 nm to about 100 nm in its medium dimension. In one embodiment, the detectable particulate ranges in size from about 1 nm to about 50 nm in its medium dimension. In another embodiment, the detectable particulate ranges in size from about 3 nm to about 50 nm in its medium dimension.

According to one embodiment of the invention, the detectable particulate has an intrinsic property that allows for monitoring, detection or imaging in vivo or in vitro. In one embodiment, the intrinsic property is derived from the core of the particulate. In another embodiment, the intrinsic property is derived from a molecular component covalently attached to the particulate.

According to one embodiment of the invention, the intrinsic property comprises paramagnetism, superparamagnetism, radioactivity, fluorescence or echogenicity. In a preferred embodiment, the intrinsic property comprises superparamagnetism.

According to one embodiment of the invention, the detectable particulate comprises monocrystalline iron oxide nanoparticles. In one embodiment, the detectable particulate is covered in a coating. In another embodiment, the coating is dextran. In yet another embodiment, the coating is cross-linked dextran. In yet another embodiment, the coating is polyethylene glycol.

According to one embodiment of the invention, the targeting member comprises an antibody that binds selectively to the target structure. In one embodiment, the antibody is polyclonal. In another embodiment, the antibody is chimeric. In another embodiment, the antibody is humanized.

According to one embodiment of the invention, the target structure is generated by infection of the cancer cell with one or more cancer-causing organisms. In one embodiment, the cancer-causing organisms comprise human polyomavirus. In another embodiment, the target structure comprises human polyomavirus T-antigen (amino acid sequence of SEQ ID NO:1). In another embodiment, the targeting member comprises an antibody that binds selectively to T-antigen from human polyomavirus. In one embodiment, the targeting member comprises pAb416.

According to one embodiment of the invention, the target structure is a protein or receptor derived from upregulation or downregulation of genes from the cancer cell. In one embodiment, the target structure comprises CD7 (amino acid sequence of SEQ ID NO:2; nucleic acid sequence of SEQ ID NO:34), CD9 (amino acid sequence of SEQ ID NO:3; nucleic acid sequence of SEQ ID NO:35), CD22 (amino acid sequence of SEQ ID NO:4; nucleic acid sequence of SEQ ID NO:36), CD25 (amino acid sequence of SEQ ID NO:5; nucleic acid sequence of SEQ ID NO:37), CD30 (amino acid sequence of SEQ ID NO:6; nucleic acid sequence of SEQ ID NO:38), CD33 (amino acid sequence of SEQ ID NO:7; nucleic acid sequence of SEQ ID NO:39), CD56 (amino acid sequence of SEQ ID NO:8; nucleic acid sequence of SEQ ID NO:40), Le^{y}, TFR (amino acid sequence of SEQ ID NO:9; nucleic acid sequence of SEQ ID NO:41), EGFR (amino acid sequence of SEQ ID NO:10; nucleic acid sequence of SEQ ID NO:42), ErbB2 (amino acid sequence of SEQ ID NO:11; nucleic acid sequence of SEQ ID NO:43), IL-4R (amino acid sequence of SEQ ID NO:12; nucleic acid sequence of SEQ ID NO:44), IL-13R (amino acid sequence of SEQ ID NO:13 and nucleic acid sequence of SEQ ID NO:45 for the alpha-1 chain; amino acid sequence of SEQ ID NO:31 and nucleic acid sequence of SEQ ID NO;48 for the alpha-2 chain) or mesothelin (amino acid sequence of SEQ ID NO:14; nucleic acid sequence of SEQ ID NO:46). In another embodiment, the targeting member comprises an antibody that binds selectively to CD7, CD9, CD22, CD25, CD30, CD33, CD56, Le^{y}, TFR, EGFR, ErbB2, IL-4R, IL-13R or mesothelin.

According to one embodiment of the invention, the antibody is attached to the surface of the particulate by a chemical linker. In one embodiment, the chemical linker comprises a thioether bond. In another embodiment, the antibody comprises a half-IgG molecule.

According to one embodiment of the invention, the blood brain barrier penetration element comprises insulin (amino acid sequence of SEQ ID NO:15; nucleic acid sequence of SEQ ID NO:47), antibodies against the human insulin receptor (amino acid sequence of SEQ ID NO:33; nucleic acid sequence of SEQ ID NO:48), Thr-Phe-Phe-Tyr-Gly-Gly-Cys-Arg-Gly-Lys-Arg-Asn-Asn-Phe-Lys-Thr-Glu-Glu-Tyr (Angiopep-1; SEQ ID NO:16), Thr-Phe-Phe-Tyr-Gly-Gly-Ser-Arg-Gly-Lys-Arg-Asn-Asn-Phe-Lys-Thr-Glu-Glu-Tyr (Angiopep-2; SEQ ID NO:17), Thr-Phe-Phe-Tyr-Gly-Gly-Ser-Arg-Gly-Lys-Arg-Asn-Asn-Phe-Arg-Thr-Glu-Glu-Tyr (Angiopep-5; SEQ ID NO:18), Thr-Phe-Phe-Tyr-Gly-Gly-Ser-Arg-Gly-Arg-Arg-Asn-Asn-Phe-Arg-Thr-Glu-Glu-Tyr (Angiopep-7; SEQ ID NO:19), Thr-Phe-Phe-Tyr-Gly-Gly-Cys-Arg-Ala-Lys-Arg-Asn-Asn-Phe-Lys-Arg-Ala-Lys-Tyr (SEQ ID NO:20), Thr-Phe-Phe-Tyr-Gly-Gly-Cys-Arg-Gly-Lys-Lys-Asn-Asn-Phe-Lys-Arg-Ala-Lys-Tyr (SEQ ID NO:21), Pro-Phe-Phe-Tyr-Gly-Gly-Cys-Arg-Gly-Lys-Arg-Asn-Asn-Phe-Lys-Thr-Glu-Glu-Tyr (SEQ ID NO:22), Thr-Phe-Phe-Tyr-Gly-Gly-Cys-Arg-Gly-Lys-Arg-Asn-Asn-Phe-Lys-Thr-Lys-Glu-Tyr (SEQ ID NO:23), Thr-Phe-Phe-Tyr-Gly-Gly-Lys-Arg-Gly-Lys-Arg-Asn-Asn-Phe-Lys-Thr-Lys-Glu-Tyr (SEQ ID NO:24), Thr-Phe-Phe-Tyr-Gly-Gly-Cys-Arg-Gly-Lys-Arg-Asn-Asn-Phe-Lys-Thr-Lys-Arg-Tyr (SEQ ID NO:25), Thr-Phe-Phe-Tyr-Gly-Gly-Lys-Arg-Gly-Lys-Arg-Asn-Asn-Phe-Lys-Thr-Ala-Glu-Tyr (SEQ ID NO:26), Thr-Phe-Phe-Tyr-Gly-Gly-Lys-Arg-Gly-Lys-Arg-Asn-Asn-Phe-Lys-Arg-Glu-Lys-Tyr (SEQ ID NO:27), Arg-Phe-Lys-Tyr-Gly-Gly-Cys-Leu-Gly-Asn-Lys-Asn-Asn-Phe-Leu-Arg-Leu-Lys-Tyr (SEQ ID NO:28), or Arg-Phe-Lys-Tyr-Gly-Gly-Cys-Leu-Gly-Asn-Lys-Asn-Asn-Tyr-Leu-Arg-Leu Lys Tyr (SEQ ID NO:29).

In one embodiment of the invention, the therapeutic agent comprises one or more of vinca alkaloids, taxanes, topoisomerase inhibitors, antitumor antibiotics, plant toxins, bacterial toxins, siRNAs, miRNAs or antisense oligonucleotides. In one embodiment, one or more of vinca alkaloids comprise vincristine, vinblastine, vinorelbine or vindesine. In another embodiment, one or more of taxanes comprise paclitaxel or docetaxel. In another embodiment, one or more of topoisomerase inhibitors comprise irinotecan, topotecan, amsacrine, etoposide or teniposide. In another embodiment, one or more of antitumor antibiotics comprise anthracyclins, bleomycin, plicamycin, mitomycin or calicheamycin. In another embodiment, one or more of the anthracyclins comprise daunorubicin, doxorubicin, epirubicin, idarubicin or valrubicin. In another embodiment, one or more of the plant toxins comprise ricin, abrin, mistletoe lectin, modeccin, pokeweed antiviral protein, bryodin 1, bouganin or gelonin. In yet another embodiment, one or more of the bacterial toxins comprise diphtheria toxin or Pseudomonas exotoxin. In another preferred embodiment, the therapeutic agent comprises siRNA raised against T antigen and agnoprotein from JC virus.

In one embodiment of the invention, the construct further comprises Rhodamine covalently attached to the construct. In one embodiment, Rhodamine is covalently attached to the targeting member.

The invention also provides a method of monitoring, detecting or imaging a cancer cell in a cell culture or a tissue, comprising exposing the cell to a construct comprising a targeting member immobilized on a detectable particulate, wherein the detectable particle has an intrinsic property that allows for monitoring, detecting or imaging of the cancer cell. Binding between the targeting member and a target structure that is expressed on the surface of the cancer cell induces internalization of the construct by the cancer cell.

The invention further provides a method of killing or preventing the growth of a cancer cell in a cell culture or a tissue, comprising exposing the cell to a construct comprising a targeting member and a therapeutic agent immobilized on a detectable particulate. Binding between the targeting member and a target structure that is expressed on the surface of the cancer cell induces internalization of the construct by the cancer cell. In one preferred embodiment, the therapeutic agent comprises one or more of vinca alkaloids, taxanes, topoisomerase inhibitors, antitumor antibiotics, plant toxins, bacterial toxins, siRNAs, miRNAs or antisense oligonucleotides.

The invention also provides a method of killing or preventing the growth of cancer cells in a subject in need thereof, the method comprising administering to the subject a therapeutically effective amount of a construct comprising a targeting member and a therapeutic agent immobilized on a detectable particulate, wherein binding between the targeting member and a target structure that is expressed on the surface of the cancer cells induces internalization of the construct by the cancer cells. In a preferred embodiment, the subject is human.

In another embodiment, the aforementioned construct is used for preparation of a medicament for the killing or prevention of growth of cancer cells in a subject in need thereof.

As envisioned in the present invention with respect to the disclosed compositions of matter and methods, in one aspect the embodiments of the invention comprise the components and/or steps disclosed therein. In another aspect, the embodiments of the invention consist essentially of the components and/or steps disclosed therein. In yet another aspect, the embodiments of the invention consist of the components and/or steps disclosed therein.

### DESCRIPTION OF FIGURES

The patent or application file contains at least one drawing executed in color. Copies of this patent or patent application publication with color drawing(s) will be provided by the Office upon request and payment of the necessary fee.

FIG. 1 shows the uptake of pAb416-CLIO conjugates by T-Antigen-expressing mouse medulloblastoma cells. pAb416 antibodies were labeled with Rhodamine. Cells were incubated with the conjugates at 37° C. in a CO₂ incubator. Panels A and D show cells incubated for 24 hours with pAb416-CLIO conjugates. Panels B and E show cells incubated only with Rhodamine-labeled CLIO (without antibody). Panels C and F show cells incubated for 2 hours with Rhodamine-labeled pAb416 (without CLIO). Top row (Panels A, B and C) show detected fluorescence derived from Rhodamine, and bottom row (Panels D, E and F) show nuclei stained with DAPI in same view as above.

FIG. 2 depicts, in graph format, the degree of internalization of pAb416-CLIO conjugates by T-antigen positive cells. T-antigen positive cells growing in wells were incubated with CLIO conjugates displaying 8 antibodies per nanoparticle. The antibodies, labeled with ¹²⁵I for detection, consisted of targeting antibodies (pAb416 or simply Ab) and non-specific mouse isotype antibodies (NSM). The x-axis registers the number of molecules of pAb416 antibody (8, 6, 4, 2 or zero) per CLIO conjugate. Internalization was determined at 1, 2, 6 and 24 hours. Control experiments used radiolabeled pAb416 and NSM not coupled to CLIO. Specificity of binding was tested by adding a 100-fold excess of pAb416 for 1 hour (see arrow) to the reaction mixture of the CLIO conjugate displaying 8 pAb416 antibodies per conjugate.

FIG. 3 depicts in graph format the time-dependent degree of internalization of antibody-CLIO conjugates by T-antigen positive cells, as the percentage of total bound conjugate. Experiments were run with pAb416-CLIO conjugates (containing 8, 6, 4, 2 or zero molecules of pAb416 antibody per nanoparticle), NSM-CLIO and non-conjugated pAb416.

FIG. 4 shows cells incubated with various concentrations of pAb416-CLIO nanoparticles for 4 h at 37 ° C. and subsequently stained with Perls' Prussian Blue for iron. Cells were counterstained with Nuclear Fast Red.

FIG. 5 shows the interaction of T-antigen positive and T-antigen negative cells with Tat-pAb416-CLIO nanoparticles, where the antibody is tagged with Rhodamine. Cells were incubated with the nanoparticles for 2 hours (left panels) or 24 hours (right panels). Top row shows Rhodamine fluorescence (seen as red) derived from Tat-pAb416-CLIO conjugate, and bottom row shows staining of nuclei with DAPI (seen as blue) in the same view.

FIG. 6 depicts in graph format the effect of the Tat peptide on binding and internalization of pAb416-CLIO nanoparticles by T-antigen positive cells. Graph A depicts the effect of the Tat peptide on total binding. Graph B depicts the effect of the Tat peptide on internalization. The x-axis for both graphs registers the ratio of pAb416 and Tat bound per CLIO nanoparticle.

### DEFINITIONS

The definitions used in this application are for illustrative purposes and do not limit the scope used in the practice of the invention.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used here, “particulate” refers to a solid particle that ranges in size from 1 nm to 1000 nm in its medium dimension, with any possible shape or form.

As used here, “detectable particulate” refers to a particulate with an intrinsic property that allows its detection and imaging in vitro or in vivo. The resolution afforded by the detection or imaging method should be sufficient to allow for spatial characterization of the particulate within the medium being analyzed. The intrinsic property that allows for detection or imaging may be derived from the core of the particulate or from a molecular component that is attached to the particulate. Examples of an intrinsic property that may allow detection or imaging of the particulate are paramagnetism, superparamagnetism, radioactivity, fluorescence and echogenicity.

As used here, the term “colloid” refers to a particulate ranging in size from 1 nm to 100 nm in its medium dimension.

As used here, “MION” refers to monocrystalline iron oxide nanoparticles. “CLIO” as used in the application refers to cross-linked dextran-coated iron oxide.

As used here, the term “blood-brain barrier penetration element” or “BBB penetration element” refers to a molecule that, once attached to a construct, enhances penetration of the construct in the brain. The blood-brain barrier penetration element may also have the ability to penetrate the brain by itself, in the absence of an attached construct or molecule. Preferentially, the element is a peptide or a protein.

As used herein, the terms “peptide,” “polypeptide” and “protein” are used interchangeably, and refer to a compound comprised of amino acid residues covalently linked by peptide bonds. A protein or peptide must contain at least two amino acids, and no limitation is placed on the maximum number of amino acids that can comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. A protein may be a receptor or a non-receptor. “Apa” is aminopentanoic acid.

As used herein, the term “fragment,” as applied to a protein or peptide, refers to a subsequence of a larger protein or peptide. A “fragment” of a protein or peptide can be at least about 10 amino acids in length; for example, at least about 50 amino acids in length; more preferably, at least about 100 amino acids in length; even more preferably, at least about 200 amino acids in length; particularly preferably, at least about 300 amino acids in length; and most preferably, at least about 400 amino acids in length.

A “nucleic acid” refers to a polynucleotide and includes polyribonucleotides and polydeoxyribonucleotides.

“Homologous”, as used herein, refers to the subunit sequence similarity between two polymeric molecules, e.g., between two nucleic acid molecules, such as two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 3′ATTGCC5′ and 3′TATGGC5′ are 50% homologous. As used herein, “homology” is used synonymously with “identity.”

“Substantially the same” amino acid sequence is defined herein as a sequence with at least 70%, preferably at least about 80%, more preferably at least about 90%, even more preferably at least about 95%, and most preferably at least 99% homology to another amino acid sequence, as determined by the FASTA search method in accordance with Pearson and Lipman, 1988, Proc. Natl. Inst. Acad. Sci. USA 85:2444-2448.

“Isolated” means altered or removed from the natural state through the actions of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as a host cell for example.

The term “antibody” is used in this disclosure to refer to an immunoglobulin, whether natural or partly or wholly synthetically produced. The term also covers any polypeptide, protein or peptide having a binding domain that is, or is homologous to, an antibody binding domain. These can be isolated from natural sources, or may be partly or wholly synthetically produced. Examples of antibodies are intact immunoglobulin molecules, as well as to fragments thereof, such as Fab, F(ab′)₂, Fv fragments, and single chain variable fragments (scFv), which are capable of binding an epitopic determinant. Antibody fragments refer to antigen-binding immunoglobulin peptides that are at least about 5 to about 15 amino acids or more in length, and that retain some biological activity or immunological activity of an immunoglobulin. Antibody as used herein includes polyclonal and monoclonal antibodies, hybrid, single chain, and humanized antibodies, as well as Fab fragments, including the products of an Fab or other immunoglobulin expression library, and suitable derivatives.

As used herein, an antibody “specifically binds”, referring to an antibody binding to a target structure, means that the antibody binds a target structure, or subunit thereof, but does not bind to a biological molecule that is not a target structure. Antibodies that specifically bind to a target structure, or subunit thereof, do not cross-react with biological molecules that are outside the target structure family. As used herein, the term “monoclonal antibody” includes antibodies that display a single binding specificity and affinity for a particular epitope. These antibodies are mammalian-derived antibodies, including murine, human and humanized antibodies. As used herein, an “antibody heavy chain” refers to the larger of the two types of polypeptide chains present in all antibody molecules in their naturally occurring conformations. As used herein, an “antibody light chain” refers to the smaller of the two types of polypeptide chains present in all antibody molecules in their naturally occurring conformations.

“Biologically active,” as used herein with respect to anti-target-structure antibodies, fragments, derivatives, homologs or analogs, means that the antibodies, fragments, derivatives, homologs or analogs have the ability to bind a target structure as described herein (e.g. anti-T-antigen, as a non-limiting example). The term “inhibit,” as used herein, means to suppress or block an activity or function by at least about ten percent relative to a control value. Preferably, the activity is suppressed or blocked by 50% compared to a control value, more preferably by 75%, and even more preferably by 95%.

As used herein, “internalization” refers to a process by which a molecule or a construct comprising a molecule binds to a target element on the outer surface of the cell membrane and the resulting complex is internalized by the cell, i.e., moves into the cytoplasma of the cancer cell without causing irreparable damage to the cell membrane. Internalization may be followed up by dissociation of the resulting complex within the cytoplasm. The target element, along with the molecule or the construct, may then undergo degradation within the cell or localize to a specific cellular compartment. Preferably, the molecule or construct is localized to the nucleus under internalization.

As used herein, “target structure” is a molecular structure located on the outer surface of the cell membrane that may interact with molecules located outside the cell. The target structure may consist of a protein or receptor, or a subunit thereof. The target structure may exist within the cell in an equivalent form that is consistent with its localization in the cytoplasm, nucleus or any other intracellular compartment. For example, the target structure may exist within the cell without the membrane localization element that allows for its localization on the outer surface of the cell membrane.

As used herein, a target structure is “preferentially expressed” in a cancer cell over a normal cell when the target structure has much lower expression in the normal cell as compared to the cancer cell, or the target structure has no expression in the normal cell but has expression in the cancer cell. The much lower expression in the normal cell should correlate with a much lower level of display of the target structure on the outer surface of the cell membrane.

“Derivative” includes any purposefully generated peptide that in its entirety, or in part, comprises an amino acid sequence substantially similar to a variable domain amino acid sequence of an antibody that binds one of the target structures contemplated in the invention. Derivatives of the antibodies of the present invention may be characterized by single or multiple amino acid substitutions, deletions, additions, or replacements. These derivatives may include: (a) derivatives in which one or more amino acid residues are substituted with conservative or non-conservative amino acids; (b) derivatives in which one or more amino acids are added; (c) derivatives in which one or more of the amino acids of the amino acid sequence used in the practice of the invention includes a substituent group; (d) derivatives in which amino acid sequences used in the practice of the invention or a portion thereof is fused to another peptide (e.g., serum albumin or protein transduction domain); (e) derivatives in which one or more nonstandard amino acid residues (e.g., those other than the 20 standard L-amino acids found in naturally occurring proteins) are incorporated or substituted into the amino acid sequences used in the practice of the invention; (f) derivatives in which one or more non-amino acid linking groups are incorporated into or replace a portion of the amino acids used in the practice of the invention; and (g) derivatives in which one or more amino acid is modified by glycosylation.

The term “target-structure-binding non-antibody molecule” indicates organic molecules or peptides that are not antibodies and that bind to one or more of the target structures that are contemplated in the invention. The target-structure-binding non-antibody molecule may bind to the target structure or a fragment of the target structure. Preferred target-structure-binding non-antibody molecules within the invention are aptamers. Aptamers are oligonucleic acid (also referred to as nucleic acid) molecules or peptide molecules that bind a specific target molecule. Nucleic acid aptamers are nucleic acid species that have been engineered through repeated rounds of in vitro selection or equivalently, SELEX (systematic evolution of ligands by exponential enrichment), to bind to various molecular targets such as small molecules, proteins, nucleic acids, and even cells, tissues and organisms. Aptamers are useful in biotechnological and therapeutic applications as they offer molecular recognition properties that rival that of the commonly used antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. See Ellington, A. D. & Szostak, J. W., 1990, “In vitro selection of RNA molecules that bind specific ligands”, Nature 346 (6287):818-22; Bock et al., 1992, “Selection of single-stranded DNA molecules that bind and inhibit human thrombin”, Nature 355 (6360):564-6; Drabovich et al., 2006, “Selection of smart aptamers by methods of kinetic capillary electrophoresis”, Anal Chem. 78 (9):3171-8, all of which are incorporated herein by reference in their entireties.

The term “anti-target-structure antibody” indicates an antibody that binds to one or more of the target structures that are contemplated in the invention. The anti-target-structure antibody may bind to the target structure or a fragment of the target structure.

“Treating”, as used herein, means ameliorating the effects of, or delaying, halting or reversing the progress of a disease or disorder. The word encompasses reducing the severity of a symptom of a disease or disorder and/or the frequency of a symptom of a disease or disorder.

“Medical intervention”, as used herein, means a set of one or more medical procedures or treatments that are required for ameliorating the effects of, delaying, halting or reversing a disease or disorder of a subject. A medical intervention may involve surgical procedures or not, depending on the disease or disorder in question. A medical intervention may be wholly or partially performed by a medical specialist, or may be wholly or partially performed by the subject himself or herself, if capable, under the supervision of a medical specialist or according to literature or protocols provided by the medical specialist.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human.

The language “effective amount” or “therapeutically effective amount” refers to a nontoxic but sufficient amount of the composition used in the practice of the invention that is effective to reduce or arrest abnormal cell growth in a subject. The desired treatment may be prophylactic and/or therapeutic. That result can be reduction and/or alleviation of the signs, symptoms, or causes of a disease or disorder, or any other desired alteration of a biological system. An appropriate therapeutic amount in any individual case may be determined by one of ordinary skill in the art using routine experimentation.

A “prophylactic” or “preventive” treatment is a treatment administered to a subject who does not exhibit signs of a disease or disorder, or exhibits only early signs of the disease or disorder, for the purpose of decreasing the risk of developing pathology associated with the disease or disorder.

A “therapeutic” treatment is a treatment administered to a subject who exhibits signs of pathology of a disease or disorder for the purpose of diminishing or eliminating those signs.

“Pharmaceutically acceptable carrier” refers herein to a composition suitable for delivering an active pharmaceutical ingredient, such as the composition of the present invention, to a subject without excessive toxicity or other complications while maintaining the biological activity of the active pharmaceutical ingredient. Protein-stabilizing excipients, such as mannitol, sucrose, polysorbate-80 and phosphate buffers, are typically found in such carriers, although the carriers should not be construed as being limited only to these compounds.

“Container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., causing the death of the targeted cancer cell in a subject.

“Applicator,” as the term is used herein, is used to identify any device including, but not limited to, a hypodermic syringe, a pipette, and the like, for administering the compounds and compositions used in the practice of the invention.

### DETAILED DESCRIPTION OF INVENTION

The present invention is based on the unexpected discovery that a construct comprising a targeting member immobilized on a detectable particulate binds to a target structure expressed on the surface of cancer cells, triggering internalization of the construct by the cancer cell. The target structure useful within the invention is preferentially expressed in cancer cells over normal cells. The construct may optionally comprise a blood brain barrier penetration element. The fact that the detectable particulate may be visualized by standard monitoring method allows for the visualization of the cancer cell either in cell culture or in vivo. The construct may also optionally comprise a therapeutic agent immobilized on the detectable particulate, wherein the therapeutic agent causes death or growth inhibition of the cancer cell.

According to the experiments discussed herein, binding of the construct comprising the targeting member immobilized on the detectable particulate to the target structure expressed on the cancer cell surprisingly triggered internalization of the construct by the cancer cell. Upon internalization, localization of the construct to the cell nucleus was also unexpectedly observed.

The present invention relates to targeting members immobilized on detectable particulates. As used in this disclosure, the term “detectable particulate” refers to a particulate with an intrinsic property that allows its detection and imaging in vitro or in vivo. The particulate preferentially has median dimensions lower than about 220 nm, forming a colloid when added to an appropriate liquid medium. More preferentially, the particulate has overall dimensions from 1 nm to 100 nm. Even more preferentially, the particulate has overall dimensions from 1 nm to 50 nm. Even more preferentially, the particulate has overall dimensions from 3 nm to 50 nm. The core of detectable particulate may be coated with a material that avoids, reduces or minimizes dissolution of the core in mostly commonly used media, such as aqueous buffers with pH between 3 and 11 and biological fluids in general. In an embodiment of the invention, the coating around the detectable particulate comprises dextran. In another embodiment, the coating is polyethylene glycol. The liquid medium in which the colloid is prepared may be water, saline, a sterile biological medium or an aqueous buffer, all of which should have pH and ionic strength values under which the detectable particulate, its coating and the targeting member are stable. A colloid suspension of the construct comprising the detectable particulate in an appropriate liquid medium may be prepared before use according to the invention, or the construct comprising the detectable particulate may be delivered as a solid material to the site of use.

The detectable particle should not be toxic by itself, and should not have the tendency to accumulate in a specific tissue or cell type by itself. It should accumulate in the desired tumor tissue in sufficiently high concentrations for unequivocal detection of the tumor-associated nanoparticles, in order to ensure high sensitivity. It should not accumulate in other tissues, in order to ensure high specificity.

The selection of the detectable particulate is intrinsically associated with the corresponding method of detection or imaging. The intrinsic property that allows for detection and imaging of the detectable particulate may be derived from the core of the particulate or from a molecular component covalently attached to the particulate.

In an embodiment of the invention, the intrinsic property of the detectable particulate is magnetism associated with paramagnetism or superparamagnetism. Paramagnetism is a form of magnetism that occurs only in the presence of an externally applied magnetic field. Paramagnetic materials are attracted to magnetic fields, having a positive magnetic susceptibility. Unlike ferromagnets, paramagnets do not retain any magnetization in the absence of an externally applied magnetic field, because thermal motion causes the spins to become randomly oriented without the applied field. Even in the presence of the field there is only a small induced magnetization because only a small fraction of the spins will be oriented by the field. Paramagnetic materials are generally characterized by atoms with unpaired spins, examples being iron oxides, uranium, platinum, tungsten and aluminum.

Superparamagnetism is another form of magnetism where the material is composed of small ferromagnetic clusters that may flip direction under thermal fluctuations. The bulk properties of such a system resemble that of a paramagnet, but on a microscopic level they are ordered. Depending on the methods used, iron oxides may be synthesized as superparamagnetic material (U.S. Pat. No. 5,262,176, incorporated by reference in this application in its entirety). For a general review on preparation and use of paramagnetic and superparamagnetic particles for biological systems, see Weissleder et al., 1990, Radiology 175:489-493.

Paramagnetic and superparamagnetic particles may be detected in cell cultures or in vivo using established electromagnetic techniques, such as magnetic resonance imaging (MRI). Magnetic resonance imaging (MRI), or nuclear magnetic resonance imaging (NMRI), is a medical imaging technique most commonly used in radiology to visualize the structure and function of the body. MRI provides great contrast between the different soft tissues of the body, making it especially useful in neurology (brain), musculoskeletal, cardiovascular, and oncology (cancer) imaging. It uses a powerful magnetic field to align the nuclear magnetization of hydrogen atoms in water molecules in the body, effectively mapping the relaxation rates of water in different tissues. MRI is often used as an imaging modality because of its high spatial resolution, even at the micrometer level, and its superior use for CNS imaging.

Paramagnetic and superparamagnetic particles induce contrast changes in MRI. MRI contrast agents do not appear directly on magnetic resonance images but affect the relaxation of surrounding hydrogen nuclei, resulting in an alteration of the intensity of the area of the image where the contrast agent is located. Contrast agents for MRI are usually based on complexes of paramagnetic metals such as gadolinium (Gd), manganese (Mn) and iron (Fe). The toxicity of Gd (III) limits its applications in MRI, requiring the use of tight complexes of high molecular weight. Monocrystalline/monodisperse iron oxide nanoparticles (MIONs) are suitable for receptor-targeted magnetic resonance contrast agents. MIONs exhibit high relaxivity and are detectable by MRI at pM levels in tissues (Jacques & Desreux, 2002, in “Topics in Current Chemistry Contrast Agents in Magnetic Resonance Imaging”, W. Krause, Ed., pp 123-164, Springer: Berlin). Iron oxide nanoparticles are non-toxic. They are eventually internalized by cells of the reticulo-endothelial system, where the iron oxide is dissolved and enters normal iron pools (Weissleder et al., 1989, Amer. J. Roentgenol. 152:167-173). MIONs are small enough to pass through inter-endothelial junctions, so they are suitable delivery vehicles for tumors.

Since MIONs are composed of iron oxide, they could undergo dissolution in slightly acidic solvents, and even in blood overtime. That would limit their stability and utility in biological systems, either in vitro or in vivo. However, MIONs may be coated with materials capable of protecting them from chemical reactions, without interfering with their magnetic properties. For example, MIONs may be coated with dextran, a complex, branched glucan (polysaccharide) made of several glucose molecules joined into chains of varying lengths (from 10 to 150 kiloDaltons). Dextran-coated MIONs have a longer residence time in the blood than uncoated MIONs (Kircher et al., 2003, Cancer Research 63:8122-8125), and this increases the length of time that the particles are available for targeting tumor cells or binding to cancer cells. Dextran-coated MIONs consist of a superparamagnetic iron oxide core, 3-5 nm in diameter, and a coating of dextran (Wunderbaldinger et al., 2002, Academic Radiology 9, Suppl 2:S304-S306). Dextran-coated MIONs may be prepared by neutralizing an aqueous solution of ferric and ferrous salts with ammonium hydroxide in the presence of dextran (Palmacci, U.S. Pat. No. 5,262,176, incorporated herein by reference in its entirety). The medium size of the MION and its degree of coating with dextran may be varied by changing the amount of iron salts, dextran and the total volume of the reaction solution. Particles of appropriate size may be isolated by utilizing filters of appropriate porosity. The resulting size of the colloidal particles may be established by various established methods, such as light scattering (Dyuzheva et al., 2002, Colloid J. 64 (1):33-38). The characterization of the iron oxide as paramagnetic or superparamagnetic may be achieved by determining the susceptibility of the material isolated (Josephson et al., 1990, Mag. Res. Imag. 8:637-646). A susceptibility of less than 5,000×10⁻⁶ c.g.s. per gram iron would indicate that the material is paramagnetic, and a susceptibility of more than 5,000×10⁻⁶ c.g.s. per gram iron would indicate that the material is superparamagnetic.

The chemical stability of the dextran coating in the dextran-coated MIONs may be increased by cross-linking The resulting material is commonly referred to as cross-linked iron oxide or CLIO. This may be achieved by treating MIONs with epichlorohydrin in the presence of a strong inorganic base, such as sodium hydroxide. This reagent reacts with the hydroxyl groups of different sugar rings, creating crosslinks that slow down metabolic processing of CLIOs.

The dextran surface of CLIO can be further modified by treatment with concentrated ammonium hydroxide or an ammonium salt in the presence of strong base. This treatment leads to the introduction of amino groups on the dextran coating (CLIO-NH₂), providing sites for convenient attachment of other molecules. CLIO-NH₂ retains the biological and physical properties of MION or CLIO (Wunderbaldinger et al., 2002, Academic Radiology 9, Suppl 2:S304-S306). The product may be characterized in terms of iron content by digesting weighed material in concentrated acid (such as hydrochloric acid) in the presence of an oxidant (such as hydrogen peroxide) and measuring the amount of ferric ion spectrophotometrically at 410 nm against a standard curve prepared from a solution of known iron concentration submitted to similar acidic oxidative conditions.

Other metal-based particles to be used within the invention as particulates detectable by MRI techniques may also be coated with appropriately stable materials or prepared by similar methods. For example, iron oxide nanoparticles may be embedded in polyacrylamide polymer (Koppelman et al., 2005, J. Magn. Magn. Materials 293:404-411; Zhang et al., 2005, J. Magn. Magn. Materials 293:193-198) or in polymeric latexes (Zheng et al., 2005, J. Magn. Magn. Materials 293:199-205). Iron oxide nanoparticles may also be covered with aminopropylsilane and coated with partially oxidized dextran (Mornet et al., 2005, J. Magn. Magn. Materials 293:127-134). An iron oxide core may be covered on a first step with a layer of 10-undecenoic acid (“inner layer”) and on a second step with a layer of PEG ester of 10-undecenoic acid (“outer layer”) (Ya ci Acar et al., 2005, J. Magn. Magn. Materials 293:1-7). Iron oxide nanoparticles may be covered by a gold metallic shell (Seino et al., 2005, J. Magn. Magn. Materials 293:144-150)—in this case, the gold metallic shell may be covalently modified with sulfhydryl-containing linkers. Iron oxide may also be partially crystallized in a calcium-silica-phosphate glass to obtain a glass-ceramic system (Eniu et al, 2005, J. Magn. Magn. Materials 293:310-313). In a similar approach, iron oxide may be embedded in the pores of silica, and amino groups may be introduced on the surface of the silica material to allow further derivatization (Grüttner et al., 2005, J. Magn. Magn. Materials 293:559-566). Iron, gadolinium or other rare earth metals nanoparticles may be encapsulated in liposomes, forming “magnetoliposomes” (Gonzales et al., 2005, J. Magn. Magn. Materials 293:265-270; Morauis et al., 2005, J. Magn. Magn. Materials 293:526-531), or may be trapped in viral-like particles (Douglas & Young, 2006, “Viruses: Making friends with old foes”, Science 312:873-875; Allen et al., 2005, “Paramagnetic viral nanoparticles as potential high-relaxivity magnetic resonance contrast agents”, Magn. Res. Med. 54:807-812; Liepold et al, 2007, “Viral capsids as MRI contrast agents”, Magn. Reson. Med. 58:871-879; Young et al., 2008, “Plant viruses as biotemplates for materials and their use in nanotechnology”, Annu Rev. Phytopathol. 46:361-384); Huang et al., 2007, “Self-assembled virus-like particles with magnetic cores”, Nano Letters 7:2407-2416; Uchida et al., 2006, “Targeting of Cancer Cells with Ferrimagnetic Ferritin Cage Nanoparticles”, J. Am. Chem. Soc. 128:16626-16633; Hosein et al., 2004, “Iron and cobalt oxide and metallic nanoparticles prepared from ferritin”, Langmuir 20:10283-10287; Flenniken et al., 2006, “Melanoma and lymphocyte cell-specific targeting incorporated into a heat shock protein cage architecture”, Chemistry & Biology 13:161-170; Suci et al., 2007, “High-density targeting of a viral multifunctional nanoplatform to a pathogenic, biofilm-forming bacterium”, Chem. & Biol. 14:387-398), dendrimeric structures (Bulte et al., 2001, “Magnetodendrimers allow endosomal magnetic labeling and in vivo tracking of stem cells”, Nature Biotechnol. 19:1141-1147; Jacques & Desreux, 2002, “New classes of MRI contrast agents”, Topics in Current Chemistry 221:123-164; Wiener et al., 1994, “Dendrimer-based metal chelates: a new class of magnetic resonance imaging contrast agents”, Magn. Reson. Med. 31:1-8), magnetoliposomes (Krack et al., 2008, J. Am. Chem. Soc. 130:7315-7320), or other metallic nanoparticles (Gao et al., 2008, “Multifunctional yolk-shell nanoparticles: A potential MRI contrast and anticancer agent”, J. Am. Chem. Soc. 130:11828-11833). Paramagnetic europium (III) and lanthanide (III) complexes may also be used in creation of detectable particulates.

In another embodiment of the invention, the intrinsic property of the detectable particulate is radioactivity, which tends to afford high sensitivity. In this case, the detectable particle comprises a radioisotope that is immobilized in a solid matrix and emits detectable radiation. An example is the radiopharmaceutical SLX804™ (Solixia, Philadelphia, Pa.). The radioactive particle would be covalently attached through sulfur atoms in the particle (by thioether linkage or disulfide linkage) to a targeting molecule. In another case, the nanoparticle may be derivatized with a linker that contains a chelating agent. For example, a nanoparticle containing free amino groups, such as CLIO-NH₂, may be reacted with the sulfosuccinimide ester of 1,4,7,10-tetraazacyclododecane-N,N′,N″,N′″-tetraacetic acid, also known as DOTA (Lewis et al., 2001, Bioconj. Chem. 12:320-324). The amino groups on the nanoparticle react with the sulfosuccinimide ester, leading to nanoparticles labeled with DOTA moieties. A similar technique using DTPA (diethylene triamine pentaacetic acid) has successfully been used with CLIO-NH₂ (Wunderbaldinger et al., 2002, Bioconj. Chem. 13:264-268). In both case, ¹¹¹Indium may be complexed by the chelating agents, generating a radiometal-chelated nanoparticles. The emitted radiation from the radiometal-chelated nanoparticles may be detected or imaged by imaging devices such as planar imaging, single proton emission computed tomography (SPECT) or positron emission tomography (PET). In the case of planar imaging or SPECT, the radiation emitted is gamma rays, and the most common isotopes used are ^{99m}Tc (half life of 6 h), ¹²³I (half life of 13 h) and ¹¹¹In/^{99m}Tc (half life of 67 h). In the case of PET, the radiation detected is a positron and the isotopes generally used have short to moderate half-lives, such as ¹¹C (half life of ˜20 min), ¹³N (half life of ˜10 min), ¹⁵O (half life of ˜2 min), ¹⁸F (half life of ˜110 min), ⁶⁸Ga (half life of 68 min), ⁶⁴Cu (half life of 12.7 h), ⁷⁶Br (half life of 16.2 h), ⁸⁶Y (half life of 14.7 h), and ¹²⁴I (half life of 4.2 days).

In another embodiment of the invention, the intrinsic property of the detectable particulate is fluorescence. Fluorescence is a kind of luminescence where the molecular absorption of a photon triggers the emission of a photon with a longer wavelength. Fluorescence may be used as an imaging property, utilizing a fluorescence microscope, confocal laser scanning microscope or total internal reflection fluorescence microscope as a detector. Due to the limited penetration of radiation in tissues, fluorescence is especially useful in cell cultures or endoscopy techniques. The particulate may comprise near infrared (NIR) tags, which have good tissue penetration (Shah and Weissleder, 2005, “Molecular optical imaging: Applications leading to the development of present day therapeutics”, NeuroRx 2:215-225). Examples include IRDye 800CW (Li-Cor Biosciences, Lincoln, Nebr.). The particulate may also comprise quantum dots. Quantum dots are semiconductor materials prepared in nanoparticle form, varying in size so that their emission wavelengths may be varied (Bakalova et al., 2007, Nat. Photonics 1:487-489). The emission of quantum dots does not bleach and does not depend on environmental characteristics. They are available commercially in a range of colors, which may be visualized simultaneously by use of a single light source. They have been used for optical imaging of tumors (Gao et al., 2004, Nat. Biotechnol. 22:969-976; Wu et al., 2003, Nat. Biotechnol. 21:41-46; Lidke et al., 2004, Nat. Biotechnol. 22:198-203; Lee et al., 2008, Phys. Chem. Chem. Phys. 10:1739-1742). Optionally the quantum dots may be coated with amphiphilic block polymers (Gao et al., Curr. Opin. Biotechnol. 16:63-72), which may improve drug delivery properties and minimize any possible toxicity associated with the core. The particulate may alternatively comprise a fluorescence molecule trapped in an insoluble medium. Generally, the fluorescence molecule of choice is green fluorescence protein (GFP), fluorescein or DyLight 488 (Spring and Davidson, “Introduction to Fluorescence Microscopy”, Nikon Microscopy U.), encapsulated in a liposome or polymer of choice.

In another embodiment of the invention, the intrinsic property of the detectable particulate is echogenicity. Echogenicity is the ability to create an echo, i.e. return a signal in ultrasound examinations. Echogenic nanoparticles have been used for targeted ultrasound imaging of cancer cells in vitro (Liu et al., 2007, Phys. Med. Biol. 52:4739-4747) and gas-loaded polylactic acid nanoparticles have been used as ultrasound contrast agents (Kwon and Wheatley, 2006, in “World Congress on Medical Physics and Biomedical Engineering—Imaging the Future Medicine”, IFMBE Proceedings Vol. 14/1, p 275, Seoul, Korea).

The invention also allows for the intrinsic property responsible for detection and imaging of the detectable particulate to be derived from a molecular component covalently attached to the particulate. In this case, the molecular component comprises a linker that anchors the molecular component to the particulate. The nature of the linker is dependent on the nature of the particulate and shall be obvious to those skilled in the art. For example, a sulfhydryl group may act as a linker for a gold particle or a particulate that presents disulfide bonds on its accessible surface. For a particulate with at least one carboxylic acid group on its accessible surface, a possible linker contains amine groups, which can be used to form amide bonds with the surface carboxylic acid groups. Furthermore, the molecular component also comprises a detectable element, used to monitor the particulate. Non-limiting examples of such detectable elements are a fluorescent molecule, such as green fluorescence protein (GFP), fluorescein or DyLight 488, all of which may be detected by fluorescence microscopy; a superparamagnetic iron oxide nanoparticle or a radionuclide trapped inside a liposome or tightly chelated by a ligand such as 1B4M-DTPA (also known as MX-DTPA or tiuxetan; Brechbiel, 2008, “Bifunctional chelates for metal nuclides”, Q. J. Nucl. Med. Mol. Imaging 52:166-173), CHX-A″-DTPA (Brechbiel, 2008), lys-DOTA (Brechbiel, 2008), EDTA (ethylenediaminetetraacetic acid), EGTA (ethyleneglycoltetraacetic acid), DMPS (2,3-dimercapto-1-propanesulfonic acid), DMSA (dimercaptosuccinic acid) or DTPA (diethylenetriaminepentaacetic acid); or an echogenic nanoparticle.

The invention includes the binding of the construct comprising a targeting member immobilized on a detectable particulate to a target structure expressed on the surface of cancer cells, wherein the targeting member is responsible for the binding to the target structure. The target structure is a molecular structure located on the surface of the cancer cell and preferentially expressed in cancer cells over normal cells. This preferential expression allows for the binding of the construct of the invention preferentially to cancer cells, allowing their detection and/or destruction. The target structure may be expressed in comparable levels on the cell surface of the most common lines of cancer cells, or may be expressed preferentially or exclusively on the cell surface of a single or a reduced number of cancer cell lines. In the former case, the construct may find utility in detecting, monitoring or eliminating non-specific cancer cell populations within cell cultures or tissues. In the later case, the construct may find utility in detecting, monitoring or eliminating specific cancer cell populations within cell cultures or tissues.

The target structure may be anchored in the lipid bilayer of the cell membrane or may be bound to the cell membrane via interaction with another structure anchored on the cell membrane. The target structure may also be presented as a self antigen or a non-self antigen by a major histocompatibility complex Class I (MHC Class I) on the cell surface.

The target structure may be derived from infection of the cell with a cancer-causing organism, such as an oncovirus, or malfunction of the cell machinery, such as upregulation of cancer-promoting genes or downregulation of cancer-suppressing genes. In any of these cases, the cancer cell produces a molecule, most likely a protein, a peptide or a receptor, which is generally not produced at all or at much lower levels in a normal cell. This protein, peptide or receptor, or a fragment thereof, finds its way to the surface of the cell, where it becomes completely or partially exposed to the extracellular environment and may be recognized by the targeting member.

The target structure may be associated with infection of the cell with a cancer-causing organism, such as T-antigen (SEQ ID NO:1), the viral regulatory protein for human polyomavirus (JC virus or JCV). Infection by JCV has been observed in multiple tumors of neural crest origin (medulloblastoma, glioblastoma, astrocytoma, and oligodendroglioma, for example), and colorectal carcinoma, where T-antigen is thought to play a key role in subverting cellular regulatory pathways. Another example of a target structure derived from a virus is HTLV-Tax (amino acid sequence of SEQ ID NO:30).

The target structure may be a protein that is characteristically expressed in cancer cells. As such, the target structure may be associated with genetic abnormalities, where oncogenes are activated and/or tumor suppressor genes are inactivated, or may be associated with cell types that multiply out of control as part of a cancer event. The target structure protein may be a receptor protein, or may be a non-receptor protein. The target structure may be expressed in normal cells but is characteristically overexpressed or activated in cancer cells and thus may be seen as a cancer marker as well. The target structure may be present on the outer surface of the cell membrane, interacting with cells and external biological molecules. The target structure may also be expressed internally and bind to cellular targets within the nucleus or in the cytoplasm, but still find its way to the outer surface of the cell membrane, effectively marking the cell as “positive” for such biological molecules. Non-limiting examples of protein or receptor target structures are CD7 (amino acid sequence of SEQ ID NO:2; nucleic acid sequence of SEQ ID NO:34), CD19 (amino acid sequence of SEQ ID NO:3; nucleic acid sequence of SEQ ID NO:35), CD22 (amino acid sequence of SEQ ID NO:4; nucleic acid sequence of SEQ ID NO:36), CD25 (amino acid sequence of SEQ ID NO:5; nucleic acid sequence of SEQ ID NO:37), CD30 (amino acid sequence of SEQ ID NO:6; nucleic acid sequence of SEQ ID NO:38), CD33 (amino acid sequence of SEQ ID NO:7; nucleic acid sequence of SEQ ID NO:39), CD56 (amino acid sequence of SEQ ID NO:8; nucleic acid sequence of SEQ ID NO:40), Le^{y}, TFR (amino acid sequence of SEQ ID NO:9; nucleic acid sequence of SEQ ID NO:41), EGFR (amino acid sequence of SEQ ID NO:10; nucleic acid sequence of SEQ ID NO:42), ErbB2 (amino acid sequence of SEQ ID NO:11; nucleic acid sequence of SEQ ID NO:43), IL-4R (amino acid sequence of SEQ ID NO:12; nucleic acid sequence of SEQ ID NO:44), IL-13R (amino acid sequence of SEQ ID NO:13 and nucleic acid sequence of SEQ ID NO:45 for alpha-1 chain; amino acid sequence of SEQ ID NO:31 and nucleic acid sequence of SEQ ID NO:48 for alpha-2 chain), and mesothelin (amino acid sequence of SEQ ID NO:14; nucleic acid sequence of SEQ ID NO:46).

A construct comprising a targeting member that binds selectively to CD7 is expected to be useful in the targeting of cancer cell associated with T-cell non-Hodgkin's lymphoma (T-NHL), in a non-limiting example.

A construct comprising a targeting member that binds selectively to CD19 is expected to be useful in the targeting of cancer cells associated with acute lymphoblastic leukemia (ALL) or B-cell non-Hodgkin's lymphoma (B-NHL), in non-limiting examples.

A construct comprising a targeting member that binds selectively to CD22 is expected to be useful in the targeting of cancer cells associated with B-cell non-Hodgkin's lymphoma (B-NHL), chronic lymphocytic leukemia (CLL) or hairy cell leukemia (HCL), in non-limiting examples.

A construct comprising a targeting member that binds selectively to CD25 or IL2R is expected to be useful in the targeting of cancer cells associated with Hodgkin's disease (HD), B-cell or T-cell non-Hodgkin's lymphoma (B-NHL or T-NHL), or leukemias in general, in non-limiting examples.

A construct comprising a targeting member that binds selectively to CD30 is expected to be useful in the targeting of cancer cells associated with Hodgkin's disease (HD), anaplastic large cell lymphoma or embryonic carcinoma, in non-limiting examples.

A construct comprising a targeting member that binds selectively to CD33 is expected to be useful in the targeting of cancer cells associated with acute myeloid leukemia (AML), in a non-limiting example.

A construct comprising a targeting member that binds selectively to CD56 is expected to be useful in the targeting of cancer cells associated with myeloma, myeloid leukemia, neuroendocrine tumors, Wilms' tumor, adult neuroblastoma, NK/T cell lymphomas, pancreatic acinar cell carcinoma, pheochromocytoma, neuroblastoma or small cell lung carcinoma, in non-limiting examples.

A construct comprising a targeting member that binds selectively to the Le^{y} polysaccharide is expected to be useful in the targeting of cancer cells derived from epithelial tissues, including breast, ovary, pancreas, prostate, esophageal, stomach, colon or non-small cell lung cancers, in non-limiting examples.

A construct comprising a targeting member that binds selectively to TFR is expected to be useful in the targeting of cancer cells associated with myeloma, myeloid leukemia, neuroendocrine tumors, Wilms' tumor, adult neuroblastoma, NK/T cell lymphomas, pancreatic acinar cell carcinoma, pheochromocytoma, neuroblastoma or small cell lung carcinoma, in non-limiting examples.

A construct comprising a targeting member that binds selectively to EGFR is expected to be useful in the targeting of cancer cells associated with breast cancer, advanced or metastatic non-small cell lung cancer, colon cancer or glioblastoma multiforme, in non-limiting examples.

A construct comprising a targeting member that binds selectively to ErbB-2 is expected to be useful in the targeting of cancer cells associated with breast cancer, ovarian cancer and stomach cancer, in non-limiting examples.

A construct comprising a targeting member that binds selectively to IL-4R is expected to be useful in the targeting of cancer cells associated with glioma, in a non-limiting example.

A construct comprising a targeting member that binds selectively to IL-13R is expected to be useful in the targeting of cancer cells associated with glioma or renal cancer, in non-limiting examples.

A construct comprising a targeting member that binds selectively to mesothelin is expected to be useful in the targeting of cancer cells associated with mesothelioma, ovarian cancer or pancreatic adenocarcinoma, in a non-limiting example.

The construct of the invention comprises an immobilized targeting member that recognizes a target structure or a fragment thereof on the surface of a cancer cell (in a cell culture or in a tissue) and binds to the target structure or the fragment thereof. The targeting member is preferentially an antibody that recognizes the target structure or a fragment thereof on the surface of a cell and binds to the target structure or fragment thereof with selectivity over other structures that may be displayed on the surface of the same cell or any other cell in the culture, the tissue or the organism under testing.

Using conventional techniques, the skilled artisan may utilize the nucleotide and amino acid sequences for the target structures listed above to prepare antigenic peptides for use in generating corresponding anti-target-structure antibodies. Alternatively, the skilled artisan may utilize commercially available antibodies against the target structures and use them within the limits of the invention. The skilled artisan may also obtain commercially available antibodies against the target structures and modify them as wished, by methods such as coupling to other antibodies, partial digestion, pegylation or covalent modification. This modified antibody may then be utilized within the limits of the invention as needed.

The antibodies used in the practice of the present invention may be polyclonal or monoclonal. Monoclonal antibodies are preferred. The antibody is preferably a chimeric antibody. For human use, the antibody is preferably a humanized chimeric antibody.

It may be appreciated that the anti-target-structure antibody used in the practice of the invention may be monovalent, divalent or polyvalent in order to achieve target structure binding. Monovalent immunoglobulins are dimers (HL) formed of a hybrid heavy chain associated through disulfide bridges with a hybrid light chain. Divalent immunoglobulins are tetramers (H2L2) formed of two dimers associated through at least one disulfide bridge.

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application Nos. WO 1993/21319 and WO 1989/09622. Functional equivalents include polypeptides with amino acid sequences substantially the same as the amino acid sequence of the variable or hypervariable regions of the antibodies raised against target integrins according to the practice of the present invention.

Functional equivalents of the anti-target-structure antibodies further include fragments of antibodies that have the same, or substantially the same, binding characteristics to those of the whole antibody. Such fragments may contain one or both Fab fragments or the F(ab′)₂ fragment. Preferably the antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Preferred constant regions are gamma 1 (IgG1), gamma 2 (IgG2 and IgG), gamma 3 (IgG3) and gamma 4 (IgG4). The light chain constant region can be of the kappa or lambda type.

The monoclonal antibodies may be advantageously cleaved by proteolytic enzymes to generate fragments retaining the target structure binding site. For example, proteolytic treatment of IgG antibodies with papain at neutral pH generates two identical so-called “Fab” fragments, each containing one intact light chain disulfide-bonded to a fragment of the heavy chain (Fc). Each Fab fragment contains one antigen-combining site. The remaining portion of the IgG molecule is a dimer known as “Fc”. Similarly, pepsin cleavage at pH 4 results in the so-called F(ab′)2 fragment.

Single chain antibodies or Fv fragments are polypeptides that consist of the variable region of the heavy chain of the antibody linked to the variable region of the light chain, with or without an interconnecting linker. Thus, the Fv comprises an antibody combining site.

Hybrid antibodies may be employed. Hybrid antibodies have constant regions derived substantially or exclusively from human antibody constant regions and variable regions derived substantially or exclusively from the sequence of the variable region of a monoclonal antibody from each stable hybridoma.

Methods for preparation of fragments of antibodies are known to those skilled in the art. See, Goding, “Monoclonal Antibodies Principles and Practice”, Academic Press (1983), p. 119-123. Fragments of the monoclonal antibodies containing the antigen binding site, such as Fab and F(ab′)2 fragments, may be preferred in therapeutic applications, owing to their reduced immunogenicity. Such fragments are less immunogenic than the intact antibody, which contains the immunogenic Fc portion. Hence, as used herein, the term “antibody” includes intact antibody molecules and fragments thereof that retain antigen binding ability.

When the antibody used in the practice of the invention is a polyclonal antibody (IgG), the antibody is generated by inoculating a suitable animal with a target structure or a fragment thereof. Antibodies produced in the inoculated animal that specifically bind the target structure are then isolated from fluid obtained from the animal. Anti-target-structure antibodies may be generated in this manner in several non-human mammals such as, but not limited to, goat, sheep, horse, rabbit, and donkey. Methods for generating polyclonal antibodies are well known in the art and are described, for example in Harlow et al. (In: Antibodies, A Laboratory Manual, 1988, Cold Spring Harbor, N.Y.). These methods are not repeated herein as they are commonly used in the art of antibody technology.

When the antibody used in the methods used in the practice of the invention is a monoclonal antibody, the antibody is generated using any well known monoclonal antibody preparation procedures such as those described, for example, in Harlow et al. (supra) and in Tuszynski et al. (Blood 1988, 72:109-115). Given that these methods are well known in the art, they are not replicated herein. Generally, monoclonal antibodies directed against a desired antigen are generated from mice immunized with the antigen using standard procedures as referenced herein. Monoclonal antibodies directed against full length or fragments of target structure may be prepared using the techniques described in Harlow et al. (supra).

The effects of sensitization in the therapeutic use of animal-origin monoclonal antibodies in the treatment of human disease may be diminished by employing a hybrid molecule generated from the same Fab fragment, but a different Fc fragment, than contained in monoclonal antibodies previously administered to the same subject. It is contemplated that such hybrid molecules formed from the anti-target-structure monoclonal antibodies may be used in the present invention. The effects of sensitization are further diminished by preparing animal/human chimeric antibodies, e.g., mouse/human chimeric antibodies, or humanized (i.e. CDR-grafted) antibodies. Such monoclonal antibodies comprise a variable region, i.e., antigen binding region, and a constant region derived from different species. By “chimeric” antibody is meant an antibody that comprises elements partly derived from one species and partly derived form at least one other species, e.g., a mouse/human chimeric antibody.

Chimeric animal-human monoclonal antibodies may be prepared by conventional recombinant DNA and gene transfection techniques well known in the art. The variable region genes of a mouse antibody-producing myeloma cell line of known antigen-binding specificity are joined with human immunoglobulin constant region genes. When such gene constructs are transfected into mouse myeloma cells, the antibodies produced are largely human but contain antigen-binding specificities generated in mice. As demonstrated by Morrison et al., 1984, Proc. Natl. Acad. Sci. USA 81:6851-6855, both chimeric heavy chain V region exon (VH)-human heavy chain C region genes and chimeric mouse light chain V region exon (VK)-human K light chain gene constructs may be expressed when transfected into mouse myeloma cell lines. When both chimeric heavy and light chain genes are transfected into the same myeloma cell, an intact H2L2 chimeric antibody is produced. The methodology for producing such chimeric antibodies by combining genomic clones of V and C region genes is described in the above-mentioned paper of Morrison et al., and by Boulianne et al. (Nature 1984, 312:642-646). Also see Tan et al. (J. Immunol. 1985, 135:3564-3567) for a description of high level expression from a human heavy chain promotor of a human-mouse chimeric K chain after transfection of mouse myeloma cells. As an alternative to combining genomic DNA, cDNA clones of the relevant V and C regions may be combined for production of chimeric antibodies, as described by Whitte et al. (Protein Eng. 1987, 1:499-505) and Liu et al. (Proc. Natl. Acad. Sci. USA 1987, 84:3439-3443).

For examples of the preparation of chimeric antibodies, see the following U.S. Pat. Nos 5,292,867; 5,091,313; 5,204,244; 5,202,238; and 5,169,939. The entire disclosures of these patents, and the publications mentioned in the preceding paragraph, are incorporated herein by reference. Any of these recombinant techniques are available for production of rodent/human chimeric monoclonal antibodies against target structures.

To further reduce the immunogenicity of murine antibodies, “humanized” antibodies have been constructed in which only the minimum necessary parts of the mouse antibody, the complementarity-determining regions (CDRs), are combined with human V region frameworks and human C regions (Jones et al., 1986, Nature 321:522-525; Verhoeyen et al., 1988, Science 239:1534-1536; Hale et al., 1988, Lancet 2:1394-1399; Queen et al., 1989, Proc. Natl. Acad. Sci. USA 86:10029-10033). The entire disclosures of the aforementioned papers are incorporated herein by reference. This technique results in the reduction of the xenogeneic elements in the humanized antibody to a minimum. Rodent antigen binding sites are built directly into human antibodies by transplanting only the antigen binding site, rather than the entire variable domain, from a rodent antibody. This technique is available for production of chimeric rodent/human anti-target structure antibodies of reduced human immunogenicity.

The antibodies molecules used in the practice of the invention may be attached to the surface of the detectable particulate by chemical modification. In the case of chemical modification, the particulate would contain “chemical linkers” on its surface and such “chemical linkers” would be reacted with chemical groups on the antibody, allowing for the covalent immobilization of the antibody on the surface of the solid support. Non-limiting examples of possible chemical groups involved in such immobilization are: a carboxylic acid group on the chemical linker, which could be reacted with an amino group on the antibody (for example, the ε-amino group on a lysine side chain) to form an amide group linking the particulate support and the antibody; an amino group on the chemical linker, which could be reacted with a carboxylic acid group on the antibody (for example, the carboxylic acid group on a glutamate or aspartate side chain) to form an amide group linking the particulate support and the antibody; a disulfide group on the chemical linker, which could be reacted with a thiol group on the antibody (for example, the thiol group on a cysteine side chain) to form a disulfide group linking the solid surface and the antibody.

One preferred mode for the immobilization of the antibody to the detectable particulate involves a two-step process. In the first step, the detectable particulate is chemically modified with amino groups and subsequently modified with maleimido groups. In the second step, the antibody bearing a sulfhydryl group is reacted with the detectable particulate bearing the maleimido group, leading to covalent attachment of the antibody to the detectable particulate. This method results in a thioether bond, which may be stable in vivo. In this particular mode, a detectable particulate such as CLIO-NH₂ (which bears amino groups on its surface) may be used, along with a bivalent reagent such as water-soluble sulfo-SMCC (sulfo-succinimidyl-4-(N-maleimidomethyl)-cyclohexane-1-carboxylate). Sulfo-SMCC contains an amine-reactive N-hydroxysuccinimide (NHS ester) and a sulfhydryl-reactive maleimide group. NHS esters react with primary amines at pH 7-9 to form stable amide bonds. Maleimides react with sulfhydryl groups at pH 6.5-7.5 to form stable thioether bonds. Sulfo-SMCC is soluble up to ˜10 mM in water and many commonly used buffers, thus avoiding the use of organic solvents which may perturb protein structure. Therefore, CLIO-NH₂ may be reacted with sulfo-SMCC, forming an amide bond and attaching a maleimido group to the CLIO-NH₂ via a 11.6 Å linker. Use of other bivalent reagents of different connectivity and geometry may give rise to inkers of different orientations and lengths. The derivatized particulate may be purified using centrifugal size exclusion columns, for example.

In this particular embodiment, the antibody should display a free sulfhydryl group on its surface, available for coupling to the maleimido group immobilized on the CLIO particulate. Two possible ways to generating such sulfhydryl group-bearing antibody would be to react amino groups on the surface of the antibody (derived from, for example, amino groups of lysine residues) with a modifying agent such as N-succinimidyl S-acetylthioacetate (SATA), or reducing a disulfide bond in the antibody with a soluble thiol such as 2-mercaptoethylamine (MEA). Since an antibody is likely to have multiple amino groups displayed on their surface, the first approach may lead to extensive and heterogeneous derivatization of the antibody. The advantage of the second approach is that it selectively reduces sulfhydryl groups located in the hinge region of the IgG, and such groups are probably not close to the antigen-binding region. As a result, the second approach results in a half-IgG molecule that can react with only one maleimido group. Reduction of the IgG may be achieved by treatment with a soluble thiol such as MEA with minimal exposure to light or air. The reduced half-IgG may be purified by size-exclusion chromatography and reacted with maleimido-bearing particulates. The ratio of half-IgGs and particulate will influence the final number of antibodies on the surface of the particulate. The excess half-IgG that does not react with the particulate may be removed by centrifugal size-exclusion methods.

The amount of antibody retained on the surface of the detectable particulate can be experimentally determined by a difference method, whereby the amount of antibody used as a reagent in the initial immobilization reaction is compared with the amount of antibody in the supernatant solution isolated after the immobilization reaction. The amount of antibody may be determined by a standard protein quantitation method, such as UV spectrophotometry or the BCA (bicinchoninic acid) assay, or any equivalent method known to those skilled in the art. The amount of antibody immobilized on the surface of the solid support may be manipulated by varying the amount of antibody used as a reagent or the contact time in the original immobilization reaction.

The present invention also relates to target-structure-binding non-antibody molecules immobilized on a detectable particulate. The target-structure-binding non-antibody molecules useful for the invention should bind to one or more of the target structures or fragments thereof. Immobilization of the target-structure-binding non-antibody molecule to the solid support should not prevent or hamper the binding of the target-structure-binding non-antibody molecule to the target structure or its fragments thereof. This requirement may be met by linking the target-structure-binding non-antibody molecule to the solid support using a method that does not considerably distort the conformation and the accessibility of the portion of the target-structure-binding non-antibody molecule that binds to the target structure. Choosing an appropriate point of attachment of the target-structure-binding non-antibody molecule to the solid support should be trivial for one skilled in the art, based on the knowledge of the putative mode of binding of the target-structure-binding non-antibody molecule to the target structure. Examples of preferred target-structure-binding non-antibody molecules are aptamers.

The construct may optionally comprise a blood-brain barrier (BBB) penetration element, which improves the ability of the construct to cross the blood-brain barrier. Such BBB-penetration element may be a peptide selected for its known blood-brain barrier-crossing properties, such as insulin. Alternatively, the BBB-penetration element may comprise antibodies to a receptor, such as antibodies to the insulin receptor or the transferrin receptor. See Pardridge, 2007, J. Controlled Release 122:345-348; which is incorporated herein in its entirety by reference. The BBB-penetration element may be anchored to the detectable particulate through “chemical linkers”. Such “chemical linkers” consist of chemical chains that are attached to the surface of the detectable particulate and display reactive chemical groups that can react with small molecules, polymeric molecules or biological molecules, so that these molecules become attached via a covalent bond to the surface of the detectable particulate. Such covalent bond may be labile or inert to standard antibody incubation media or bodily fluids.

Non-limiting examples of BBB-penetration elements are insulin (amino acid sequence of SEQ ID NO:15; nucleic acid sequence of SEQ ID NO:47), antibodies against the human insulin receptor (amino acid sequence of SEQ ID NO:33; nucleic acid sequence of SEQ ID NO:48), Thr-Phe-Phe-Tyr-Gly-Gly-Cys-Arg-Gly-Lys-Arg-Asn-Asn-Phe-Lys-Thr-Glu-Glu-Tyr (Angiopep-1; SEQ ID NO:16), Thr-Phe-Phe-Tyr-Gly-Gly-Ser-Arg-Gly-Lys-Arg-Asn-Asn-Phe-Lys-Thr-Glu-Glu-Tyr (Angiopep-2; SEQ ID NO:17), Thr-Phe-Phe-Tyr-Gly-Gly-Ser-Arg-Gly-Lys-Arg-Asn-Asn-Phe-Arg-Thr-Glu-Glu-Tyr (Angiopep-5; SEQ ID NO:18), Thr-Phe-Phe-Tyr-Gly-Gly-Ser-Arg-Gly-Arg-Arg-Asn-Asn-Phe-Arg-Thr-Glu-Glu-Tyr (Angiopep-7; SEQ ID NO:19), Thr-Phe-Phe-Tyr-Gly-Gly-Cys-Arg-Ala-Lys-Arg-Asn-Asn-Phe-Lys-Arg-Ala-Lys-Tyr (SEQ ID NO:20), Thr-Phe-Phe-Tyr-Gly-Gly-Cys-Arg-Gly-Lys-Lys-Asn-Asn-Phe-Lys-Arg-Ala-Lys-Tyr (SEQ ID NO:21), Pro-Phe-Phe-Tyr-Gly-Gly-Cys-Arg-Gly-Lys-Arg-Asn-Asn-Phe-Lys-Thr-Glu-Glu-Tyr (SEQ ID NO:22), Thr-Phe-Phe-Tyr-Gly-Gly-Cys-Arg-Gly-Lys-Arg-Asn-Asn-Phe-Lys-Thr-Lys-Glu-Tyr (SEQ ID NO:23), Thr-Phe-Phe-Tyr-Gly-Gly-Lys-Arg-Gly-Lys-Arg-Asn-Asn-Phe-Lys-Thr-Lys-Glu-Tyr (SEQ ID NO:24), Thr-Phe-Phe-Tyr-Gly-Gly-Cys-Arg-Gly-Lys-Arg-Asn-Asn-Phe-Lys-Thr-Lys-Arg-Tyr (SEQ ID NO:25), Thr-Phe-Phe-Tyr-Gly-Gly-Lys-Arg-Gly-Lys-Arg-Asn-Asn-Phe-Lys-Thr-Ala-Glu-Tyr (SEQ ID NO:26), Thr-Phe-Phe-Tyr-Gly-Gly-Lys-Arg-Gly-Lys-Arg-Asn-Asn-Phe-Lys-Arg-Glu-Lys-Tyr (SEQ ID NO:27), Arg-Phe-Lys-Tyr-Gly-Gly-Cys-Leu-Gly-Asn-Lys-Asn-Asn-Phe-Leu-Arg-Leu-Lys-Tyr (SEQ ID NO:28), and Arg-Phe-Lys-Tyr-Gly-Gly-Cys-Leu-Gly-Asn-Lys-Asn-Asn-Tyr-Leu-Arg-Leu Lys Tyr (SEQ ID NO:29).

The construct may optionally comprise a therapeutic agent that causes killing or growth inhibition of the target cancer cell. The therapeutic agent may have no or limited capacity of penetrating a cell (or the brain) by itself, but the construct of the invention comprising the therapeutic agent has the ability of binding to one or more target structures located on the surface of the cancer cell and undergo internalization. The specific or selective internalization of the construct comprising the therapeutic agent by cancer cells allows for the therapeutic agent to interfere specifically or at least selectively with the metabolism or catabolism of cancer cell, causing their death or inhibiting their growth. The preferred therapeutic agents in the invention are organic drugs, proteins, peptides siRNAs, miRNAs and antisense oligonucleotides. Such agents are known to interfere with cellular function and may be particularly potent against cancer cells due to the greater dependency of cancer cells on specific metabolic pathways, to the greater rate of multiplication of cancer cells or to the lower ability of cancer cells to recover from therapeutic challenges.

The therapeutic agent may be attached to the construct of the invention through a covalent bond. The covalent bond may involve on one side a chemical group on the therapeutic agent or on a modified version of the therapeutic agent, and on the other side a chemical group on the construct. Examples of covalent bonds are amide bonds (derived from the coupling on an amino group to a carboxylate or acyl chloride group), amine bonds (derived from the displacement of a halide by an amine group, or from the reductive alkylation of a ketone or aldehyde with an amine in the presence of a reducing agent) or disulfide bonds (derived from the oxidative coupling of two sulfhydryl groups). The covalent bond may be stable to most chemical and biochemical environments or may be engineered to undergo cleavage once internalized by the cell, due to specific conditions existing inside the cancer cell over the extracellular media. For example, a disulfide covalent bond may undergo cleavage inside the cancer cell because it is unstable to the usually reducing conditions within a cancer cell, where the concentration of glutathione is much higher than in blood plasma. The therapeutic agent may be anchored to the detectable particulate through “chemical linkers”. Such “chemical linkers” consist of chemical chains that are attached to the surface of the detectable particulate and display reactive chemical groups that can react with small molecules, polymeric molecules or biological molecules, so that these molecules become attached via a covalent bond to the surface of the detectable particulate. Such covalent bond may be labile or inert to standard antibody incubation media or bodily fluids.

Examples of organic drugs that may be used as therapeutic agents are vinca alkaloids, taxanes, topoisomerase inhibitors, and antitumor antibiotics. Vinca alkaloids, such as vincristine (Oncovin™), vinblastine, vinorelbine (Navelbine™) and vindesine (Eldisine™), bind to tubulin, inhibiting the assembly of tubulin into microtubules during the M phase of the cell cycle. Vinca alkaloids are used to treat Hodgkin's lymphoma, leukemia, nephroblastoma, melanoma, non-small cell lung cancer, breast cancer, head and neck cancer, and testicular cancer. Taxanes, such as paclitaxel (Taxol™) and docetaxel (Taxotere™), stabilize tubulin structures during cell division, preventing the separation of chromosomes during anaphase. Taxanes are used to treat lung cancer, ovarian cancer, breast cancer, head and neck cancer, colorectal cancer, prostate cancer, liver cancer, renal cancer, gastric cancer, melanoma and advanced forms of Kaposi's sarcoma. Topoisomerase inhibitors, such as irinotecan (Camptosar™), topotecan (Hycamtin™), amsacrine, etoposide (Eposin™) and teniposide (Vumon™), inhibit type I or type II topoisomerases and interfere with DNA transcription and replication by upsetting DNA supercoiling. Topisomerase inhibitors are used to treat Ewing's sarcoma, lung cancer, testicular cancer, lymphoma, non-lymphocytic leukemia, glioblastoma multiforme, ovarian, colon, acute lymphoblastic/lymphocytic leukemia cancer. Antitumor antibiotics, such as anthracyclins, bleomycin, plicamycin, mitomycin and calicheamycin, are antibiotics with cytotoxic properties. Anthracyclins, such as daunorubicin (Daunomycin™), doxorubicin (Adriamycin™), epirubicin (Ellence™), idarubicin (Idamycin™) and valrubicin (Valstar™), inhibit DNA and RNA synthesis, inhibit topoisomerase II enzyme and create iron-mediated free radicals inside the cell. These compounds are used to treat a wide range of cancers, including leukemias, lymphomas, and breast, uterine, bladder, ovarian and lung cancers. Bleomycin (Blenoxane™) acts by inducing DNA strand breaks and is used in the treatment of Hodgkin's lymphoma, squamous cell carcinomas, and testicular cancer. Plicamycin (Mithramycin™) has been used in the treatment of testicular cancer. Mitomycin, a potent DNA crosslinker, is used for treating upper gastro-intestinal cancers, breast cancers and bladder cancers. Calicheamicin, a DNA cleaver, is used for treatment of acute myelogeneous leukemia.

Examples of proteins that may be used as therapeutic agents are plant and bacterial toxins. Examples of plant toxins are holotoxins (class II ribosome-inactivating proteins) such as ricin, abrin, mistletoe lectin and modeccin, and hemitoxins (class I ribosome-inactivating proteins) such as pokeweed antiviral protein, bryodin 1, bouganin and gelonin. Examples of bacterial toxins are diphtheria toxin, and Pseudomonas exotoxin.

An example of siRNA that be used as therapeutic agents is an siRNA raised against T antigen and agnoprotein from JC virus, as described by Radhakrishnan et al. (J. Virol. 2004, 7264-7269). This siRNA was prepared by Dharmacon, Inc. (Chicago, Ill.), according to the specifications outlined in the article by Radhakrishnan et al.

The ability of the construct of the invention to be internalized by cancer cells may be conveniently determined by fluorescence methods in the case that the construct comprises an antibody labeled by a fluorescence probe, such as Rhodamine. Such labeling may be achieved by treating the antibody with a reagent such as NHS-Rhodamine, where Rhodamine is covalently attached to N-hydroxy-succinamide. The ratio between the amounts of antibody and NHS-Rhodamine, the reaction time and the reaction conditions will determine the average number of Rhodamines coupled to the antibody. This average number may be determined spectrophotometrically by measuring the absorbances at 280 nm for protein and at 565 nm for Rhodamine, and calculating the concentrations of protein and Rhodamine based on the corresponding extinction coefficients.

The Rhodamine-labeled construct may be contacted with a cell culture or an in vivo tissue for an appropriate amount of time, after which fluorescence microscopy may be used to determine whether the construct penetrated the cell. Presence of the construct in the nucleus may be further investigated by comparing the fluorescence microscopy image obtained with Rhodamine to that obtained with DAPI, a known nucleus stain.

### Medical and Therapeutic Use and Pharmaceutical Compositions

The compositions of the invention find utility in the imaging and detection of cancer cells in cell cultures and in vivo. The compositions of the invention also find utility in the imaging and detection of cancer cell in organs and tissues ex vivo. The compositions of the invention also find utility in the killing or growth prevention of cancer cells, provided that the compositions include a therapeutic agent that is capable of killing or stopping the growth of cancer cells once internalized by them.

Selectivity for a specific type of cancer cell or a specific group of types of cancer cells is conferred by the targeting member present in the construct, since the targeting member binds selectively to a target structure present on the surface of a specific type of cancer cell or a specific group of types of cancer cells. Therefore, the choice of targeting member incorporated on the construct will ultimately help determine the specificity of the construct. By identifying the target structure of interest based on the knowledge available about cancer cells and their biological structure, one skilled in the art would be able to choose the desired targeting member that binds the target structure selectively.

When used to detect or image cancer cells in a cell culture, one skilled in the art should be able to vary the exposure time, the amount of construct and the final concentration of the construct to optimize the detection or imaging desired. Other experimental parameters may be varied to achieve the other effect, depending on the specific experiment conducted, and identification of such parameters should involve minimal experimentation by those skilled in the art.

The compositions of the invention are particularly amenable to dermatological treatments in those cases where detection of cancer cells or killing of cancer cells in the skin or nearby tissues is involved. In such cases, the compositions of the present invention can be introduced at or near the tissue involved. This may be accomplished in different ways. A fine particle dispersion of the composition of the invention may be injected cutaneously or subcutaneously. A suitably shaped form of the composition of the invention may be implanted under the skin. The composition of the invention may alternatively be presented in suitable form for external application, such as patches, or may presoaked in a medium, such as cloth or cotton, and applied to the skin.

It should be appreciated that all the preceding and following therapeutic applications may also be performed in an “ex vivo” manner. In this case, a tissue or organ in which detection or killing of cancer cells is desired may be removed from an organism, under conditions which allows the tissue or organ to remain viable and with minimal alteration of the natural conditions of the tissue or organism. The procedure should usually be conducted under sterile conditions to minimize possibility of contamination. The tissue or organ may be exposed to the composition of the invention for a variable amount of time, from minutes to days. The compositions of the invention may be provided as suspensions, powders, pastes or other suitable presentations, and the mode of contact between the composition of the invention and the tissue or organ should be such that detection or killing of cancer cells is achieved. Those skilled in the art should be able to determine the optimal contact time without undue experimentation. Once the desired detection or killing of cancer cells is achieved, the tissue or organ may be returned to the original organism or to another organism in need to such tissue or organ. Transplantations should proceed following the procedures known by those skilled in the art.

One skilled in the art can readily determine an effective amount of construct comprising the targeting member to be administered to a given subject, by taking into account factors such as the size and weight of the subject; the extent of disease penetration; the age, health and sex of the subject; the route of administration; and whether the administration is local or systemic. In the case where the construct comprises a therapeutic agent meant to selectively kill cancer cells, the amount of construct comprising the targeting member and the therapeutic agent to be administered to a subject depends upon the mass of cancer cells, the location and accessibility of the cancer cells, and the degree of killing of cancer cells caused by the therapeutic agent. Those skilled in the art may derive appropriate dosages and schedules of administration to suit the specific circumstances and needs of the subject. For example, suitable doses of construct comprising the targeting member and the therapeutic agent to be administered can be estimated from the volume of cancer cells to be killed. Typically, dosages of construct comprising the targeting member and the therapeutic agent are between about 0.001 mg/kg and about 15 mg/kg body weight. In some embodiments, dosages are between about 0.01 mg/kg and about 10 mg/kg body weight.

It is understood that the effective dosage will depend on the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation.

A mixture of constructs of the invention can be administered in equimolar concentrations to a subject in need of such treatment. The mixture may comprise, for example, detectable particulates attached to different antibodies to the same target structure or antibodies to different target structures. The mixture may also comprise, for example, detectable particulates attached to different targeting members that bind to the same target structure or different target structures. In another instance, the constructs of the invention are administered in concentrations that are equimolar. In another instance, the constructs of the invention are administered in concentrations that are not equimolar. In other instance, the constructs of the invention are administered as equal amounts of protein, by weight, per kilogram of body weight. In another instance, the constructs of the invention are administered in unequal amounts. In yet other instances, the amount of each construct of the invention to be administered is based on its killing or neutralizing activity.

In general, the schedule or timing of administration of a mixture of the constructs of the invention is according to the accepted practice for the procedure being performed.

When used in vivo, the constructs of the invention are preferably administered as a pharmaceutical composition, comprising a mixture, and a pharmaceutically acceptable carrier. The constructs of the invention may be present in a pharmaceutical composition in an amount from 0.001 to 99.9 wt %, more preferably from about 0.01 to 99 wt %, and even more preferably from 0.1 to 95 wt %.

All of the various constructs of the invention to be administered need not be administered together in a single composition. The different constructs can be administered in separate compositions. For example, if three different constructs comprising different targeting members are to be administered, the three different constructs can be delivered in three separate compositions. In addition, each construct can be delivered at the same time, or the constructs can be delivered consecutively with respect to one another. Thus, the mixture of the constructs can be administered in a single composition, or in multiple compositions comprising one or more constructs.

In view of the disclosure contained herein, those skilled in the art will appreciate that the present compositions are capable of having a beneficial effect in many kinds of cancer, such as, but not limited to, brain tumors, leukemia, melanoma, lung cancer, stomach cancer, colon cancer and pancreatic cancer. It is therefore contemplated that the compositions of this invention may take numerous and varied forms, depending upon the particular circumstance of each application. For example, the construct used in the practice of the invention may be incorporated into a solid pill or may in the form of a liquid dispersion or suspension. In general, therefore, the compositions of the present invention preferably comprise the construct and a suitable, non-toxic, physiologically acceptable carrier. As the term is used herein, “carrier” refers broadly to materials that facilitate administration or use of the present compositions for cell imaging, cancer treatment or “ex vivo” procedures. A variety of non-toxic physiologically acceptable carriers may be used in forming these compositions, and it is generally preferred that these compositions be of physiologic salinity.

For some applications involving cancer treatment, it may be desirable to have available a physically applicable or implantable predetermined solid form of material containing the composition of the invention. Accordingly, it is contemplated that the compositions of this invention may be incorporated in solid forms such as rods, needles, sheets, gels or pastes. They may thus be introduced at or near the sites of cancer growth, for example. In such embodiments, the compositions of the present invention are preferably combined with a solid carrier that itself is bio-acceptable and suitably shaped for its use. For many applications, it is preferred that the compositions of the present invention be prepared in the form of an aqueous dispersion, suspension or paste that can be directly applied to the site of cancer growth. To prepare these compositions, a detectable particulate comprising a targeting member and a therapeutic agent can be used in the condition in which is it available after preparation, with optional addition of other components, including a fluid carrier, such as saline water. The derivatized particulate support solid may also be dried, milled, or modified to a desired particle size or solid form before therapeutic use. The particle size can be optimized for the intended therapeutic use of the composition. The particulate ranges in size from about 1 nm to 1 cm. In a more preferred embodiment, the particulate support ranges in size from about 1 nm to about 0.1 mm. In a preferred embodiment, the carrier is an aqueous medium and the compositions are prepared in the form of an aqueous suspension of detectable particulate comprising a targeting member and a therapeutic agent.

The anti-target-structure antibody-coated particulates, or pharmaceutical compositions comprising these compounds, may be administered by any method designed to allow compounds to have a physiological effect. Administration may occur enterally or parenterally; for example orally, rectally, intracisternally, intravaginally, intraperitoneally or locally. Parenteral and local administrations are preferred. Particularly preferred parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, intraperitoneal injection, intracranial and intrathecal administration for CNS tumors, and direct application to the target area, for example by a catheter or other placement device. Particularly preferred local administrations include powders, ointments, suspensions and drops.

The compositions of the present invention are useful for prophylactic and/or therapeutic treatment. The pharmaceutical compositions can be administered in a variety of unit dosage forms depending upon the method of administration.

The pharmaceutical compositions of this invention are particularly useful for parenteral administration, such as administration into a body cavity or lumen of an organ. The compositions for administration will commonly comprise a suspension of the anti-target-structure antibody-coated particulate in a pharmaceutically acceptable carrier, preferably an aqueous carrier. A variety of aqueous carriers can be used, e.g., buffered saline and the like. These suspensions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The amount of the anti-target-structure antibody-coated particulate in these formulations can vary widely, and will be selected primarily based on fluid volumes, viscosities, body weight and the like in accordance with the particular mode of administration selected and the subject's needs.

Thus, a typical pharmaceutical composition for intravenous administration would be about 0.1 to 10 mg per subject per day. Dosages from 0.1 up to about 100 mg per subject per day may be used, particularly when the drug is administered to a secluded site and not into the blood stream, such as into a body cavity or into a lumen of an organ. Methods for preparing parenterally administrable compositions will be known or apparent to those skilled in the art and are described in more detail in such publications as Remington's Pharmaceutical Science, 15th ed., 1980, Mack Publishing Company, Easton, Pa.

The compositions containing the anti-target-structure antibody-coated particulate of the invention can be administered for therapeutic treatments. In therapeutic applications, preferred pharmaceutical compositions are administered in a dosage sufficient to kill or stop growth of cancer cells. An amount adequate to accomplish this is defined as a “therapeutically effective dose.” Amounts effective for this use will depend upon the severity of the disease and the general state of the subject's health.

Single or multiple administrations of the compositions may be administered depending on the dosage and frequency as required and tolerated by the subject. In any event, the administration regime should provide a sufficient quantity of the composition of this invention to effectively treat the subject.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Pharmaceutical compositions that are useful in the methods used in the practice of the invention may be prepared, packaged, or sold in formulations suitable for oral, rectal, intracisternal, intravaginal, intraperitoneal or local, or another route of administration. Other contemplated formulations include projected nanoparticles, liposomal preparations, resealed erythrocytes containing the active ingredient, and immunologically-based formulations.

A pharmaceutical composition used in the practice of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition used in the practice of the invention will vary, depending upon the identity, size, and condition of the subject treated and further depending upon the route by which the composition is to be administered. By way of example, the composition may comprise between 0.01% and 99.9% (w/w) active ingredient.

Controlled- or sustained-release formulations of a pharmaceutical composition used in the practice of the invention may be made using conventional technology.

A formulation of a pharmaceutical composition used in the practice of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid comprises a carbon-containing liquid molecule that exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture.

Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition used in the practice of the invention that are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropyl methylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Powdered and granular formulations of a pharmaceutical preparation used in the practice of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition used in the practice of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (e.g. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (e.g. powder or granular) form for reconstitution with a suitable vehicle (e.g. sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or diglycerides. Other usual parentally-administrable formulations include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions used in the practice of the invention are known in the art and described, for example in Remington's Pharmaceutical Sciences (Genaro, ed. Mack Publishing Co., 1985, Easton, Pa.), which is incorporated herein by reference.

The pharmaceutical compositions of the invention may be dispensed to the subject under treatment with the help of an applicator. The applicator to be used may depend on the specific medical condition being treated, amount and physical status of the pharmaceutical composition, and choice of those skilled in the art.

The pharmaceutical compositions of the invention may be provided to the subject or the medical professional in charge of dispensing the composition to the subject, along with instructional material. The instructional material includes a publication, a recording, a diagram, or any other medium of expression, which can be used to communicate the usefulness of the composition and/or compound used in the practice of the invention in a kit. The instructional material of the kit may, for example, be affixed to a container that contains the compound and/or composition used in the practice of the invention or may be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

### Exemplification Used in the Practice of the Invention

The practice used in the practice of the invention is illustrated by the following non-limiting examples.

### Materials

Monocrystalline/monodisperse iron oxide nanoparticles coated with polysaccharides may be synthesized according to the method of Palmacci (U.S. Pat. No. 5,262,176, incorporated herein by reference in its entirety).

Dextran T-10, a high purity dextran fraction with a normative molecular weight of 10,000 Daltons, is available from Pharmacosmos (Holbaek, Denmark).

Sulfosuccinimidyl-4-(N-maleimidomethyl)-cyclohexane-1-carboxylate (Sulfo-SMCC), a water-soluble, non-cleavable and membrane impermeable crosslinker, is available from ThermoScientific (Rockford, Ill.).

CC2-coated glass chamber slides (Nunc™ Lab Tek™) are available from Sigma Aldrich. CC2 coating consists of a chemically modified growth surface that provides binding sites optimal for fastidious cells (e.g. neurons), remains stable without refrigeration and mimics polylysine.

pAb416, a monoclonal antibody against T-antigen antibody, may be obtained from EMD Bioscience (San Diego, Calif.) as a solution containing 0.2 mg IgG per ml and 0.2% gelatin and 0.01% sodium azide in 50 mM phosphate, pH 7.5.

The peptide “Fl-Tat”, Gly-Arg-Lys-Lys-Arg-Arg-Gln-Arg-Arg-Arg-Gly-Tyr-Lys(fluorescein)-Cys (amino acid sequence of SEQ ID NO:32), where the Lys residue closest to the C-terminus is coupled by its ε-amino group to fluorescein via an amide bond, was synthesized by BioPeptide Co., Inc. (San Diego, Calif.).

### Methods

### Example 1

### Preparation of Dextran-Coated Superparamagnetic Iron Oxide Particulates

In a typical procedure to prepare a dextran-covered colloid (adapted from Palmacci, 2003), 1005 milliliters of a 0.2-μm filtered aqueous solution of 450 grams of dextran T-10, and 31.56 grams (116.76 mmoles) of ferric chloride hexahydrate is cooled to 2-4° C. To the above cooled mixture is added a freshly prepared (within 15-30 minutes of use) 0.2 μm-filtered aqueous solution containing 12.55 gram (63.13 mmoles) of ferrous chloride tetrahydrate dissolved in water to a total volume of 43 milliliters. While being rapidly stirred, the above acidic solution is neutralized by the dropwise addition of 45 milliliters of 28-30% ammonium hydroxide solution cooled to 2-4° C. The greenish suspension is then heated to between 75° and 85° C. for an hour. The mixture is maintained in this temperature range for 75 minutes while being stirred constantly. The ammonium chloride, along with excess dextran and ammonium hydroxide are removed by ultrafiltration on a 2 liter, CH-2 apparatus (Amicon, Inc., Danvers, Mass.) equipped with 300 kD hollow fiber cartridges. After about six washes, the eluent is found to be free of all contaminants. The colloidal product is concentrated by ultrafiltration (<40 mg/ml) and 0.2 μm filtered.

The resulting homogeneous dextran-covered colloid exhibits a size of roughly 10-20 nm and a susceptibility of greater than 25,000×10⁻⁶ (c.g.s.) per gram iron, which indicates the iron is superparamagnetic. See Josephson et al., (1990) Mag. Res. Imag. 8, pp. 637-646, for details of susceptibility measurement. Paramagnetic iron would have a susceptibility of less than 5,000×10⁻⁶ (c.g.s.) per gram iron.

### Example 2

### Preparation of Dextran-Coated Superparamagnetic Iron Oxide Particulates

In another typical procedure to prepare a dextran-covered colloid (adapted from Palmacci, 2003), 381 milliliters of a 0.2-μm filtered aqueous solution of 170.5 grams of dextran T-10, and 31.56 grams (116.76 mmoles) of ferric chloride hexahydrate is cooled to 2-4° C. To the above cooled mixture is added a freshly prepared (within 15-30 minutes of use) 0.2-μm filtered aqueous solution containing 12.55 grams (63.13 mmoles) of ferrous chloride tetrahydrate dissolved to a total volume of 43 milliliters. While being rapidly stirred, the above acidic solution is neutralized by the dropwise addition of 28-30% ammonium hydroxide solution cooled to 2-4° C. The greenish suspension is then heated to between 75° and 85° C. over an one-hour heating interval. The mixture is maintained in this temperature range for 75 minutes while being stirred constantly. The ammonium chloride, along with excess dextran and ammonium hydroxide, are removed by ultrafiltration on a 2 liter, CH-2 apparatus (Amicon, Inc., Danvers, Mass.) equipped with 300 kD hollow fiber cartridges. After about six washes, the eluent is found to be free of all contaminants. The colloidal product is concentrated by ultrafiltration (<40 mg/ml) and is subsequently passed through filters of decreasing porosity of 800 nm, 450 nm and 220 nm.

The resulting homogeneous dextran-covered colloid exhibits a size of roughly 30-50 nm and a susceptibility of greater than 25,000×10⁻⁶ (c.g.s) per gram iron, which indicates the iron is superparamagnetic.

### Example 3

### Stabilization of Dextran Coating on Dextran-Coated Superparamagnetic Iron Oxide Particulates: Generation of CLIO-NH₂

The dextran coat of the particles may be stabilized by crosslinking according to Palmacci (2003). Briefly, 0.89 mL of dextran-coated iron oxide colloid (0.18 mmol iron) was diluted with 1.5 ml of 5M sodium hydroxide, after which 0.6 mL epichlorohydrin was added. Amine groups were introduced onto the surface of the particles by treatment with concentrated ammonium hydroxide (1.76 mL), followed by heating at 37° C. overnight. The final product was dialyzed against water using dialysis tubing with 12-14 kDalton molecular weight cutoffs. After aeration for 24 hours, the colloid was dialyzed and concentrated in a centrifugal concentrator with a molecular weight cutoff of 30 kDaltons.

The concentration of iron in the final product was confirmed using a spectrophotometric method (Moore et al., 2001, Radiology 221:244-250). Specifically, 10 μL of product was added to 1 mL of 6 M HCl, 0.3% hydrogen peroxide and incubated at room temperature for 1 hour. The absorbance of this solution at 410 nm was measured in a Pharmacia UV/Vis spectrophotometer with 6 M HCl, 0.3% hydrogen peroxide as the blank. The concentration of iron in the solution was determined by comparison with a standard curve prepared from serially diluted solutions of 200 mM ferrous ammonium sulfate reacted as above with 6M HCl, 0.3% hydrogen peroxide.

### Example 4

### Conjugation of Rhodamine to T-Antigen Antibody: Generation of Rhodamine-pAb416

The monoclonal antibody specific for T-antigen (pAb416) IgG was labeled with Rhodamine using an N-hydroxysuccinimidyl (NHS) ester for coupling to primary amino groups. Briefly, pAb416 antibody was obtained from EMD Calbiochem as a solution containing 0.2 mg IgG per ml and 0.2% gelatin in 50 mM phosphate, pH 7.5. The antibody solution was concentrated by centrifugation at 14,000×g in a centrifugal concentrator (MicroCon YM30) with molecular weight cutoff of 30,000 Daltons. The final concentration of IgG in the solution was 3.14 mg/mL, as determined by measuring absorbance at 280 nm.

NHS-Rhodamine (13.3 nmol in 2.8 μl DMSO) was added to the IgG solution (1.33 nmol in 65 μL) and reacted at 4° C. overnight, protected from light. The unbound Rhodamine was then removed by centrifugal concentrator (MicroCon YM30), by adding 370 μl 50 mM PBS, pH 7.2 and concentrating to 50 μl three times. The final volume was adjusted to 420 μl with PBS, pH 7.2. The number of Rhodamines bound to each molecule of IgG was determined spectrophotometrically by measuring the absorbances at 280 nm for protein and at 565 nm for Rhodamine. This was also confirmed on size-exclusion HPLC using Bio-Silect SEC400 column, eluted with 100 mM phosphate, 150 mM NaCl, pH 6.8, on which the peak of IgG could be separated from the peak of gelatin. On average 3 Rhodamine molecules were coupled to each IgG.

### Example 5

### Conjugation of Rhodamine-pAb416 Monoclonal Antibody to Nanoparticles

In this example, antibodies were coupled to amine-containing nanoparticles using three steps. In Step 1, the water-soluble sulfo-SMCC [sulfo-succinimidyl-4-(N-maleimidomethyl)-cyclohexane-1-carboxylate] was coupled to the amine groups located on the CLIO-NH₂ nanoparticles. In Step 2, an antibody derivative with a free sulfhydryl group was generated. In Step 3, the antibody derivative with a free sulfhydryl group was reacted with the maleimido-bearing nanoparticle. This sequence of steps resulted in the antibody being coupled to the nanoparticle through a thioether bond.

### Step 1: Derivatization of CLIO-NH₂ Nanoparticles to CLIO-Maleimide Nanoparticles.

The nanoparticles CLIO-NH₂ (0.5 nmol particles, 57.6 μg Fe, suspended in 30 uL bicarbonate buffer, pH 8.0) were diluted with 15 μl 0.1M phosphate, pH 7.4, treated with sulfo-SMCC (50 nmol in 5 μl in water) and allowed to react for 2 hr at room temperature. After reacting for 2 hr at room temperature, the nanoparticles were purified using a centrifugal size exclusion column (BioGel P-6, pre-equilibrated in 0.1M phosphate, pH 7.0), yielding modified CLIO-maleimide_nanoparticles.

### Step 2: Generation of a Free Sulfhydryl-Bearing Antibody

In this particular example, the internal disulfides of Rhodamine-pAb416 antibody were reduced with 2-mercaptoethylamine (MEA), yielding two half-IgG molecules.

Specifically, to 1.25 nmol of Rhodamine-IgG in 55 uL of PBS buffer (pH 7.2 with 5 mM EDTA) were added 5.5 μl of 2-MEA (60 mg/ml freshly dissolved in PBS pH 7.2 with 5 mM EDTA). The reagents were allowed to react at 37° C. in the dark for 90 minutes. Then, the excess 2-MEA was removed from antibody by size-exclusion chromatography on BioGel P-6 desalting column (pre-equilibrated in PBS pH 6.5, 1 mM EDTA and precoated with 0.2% gelatin). The void volume peak eluted from the column was immediately used for coupling to modified nanoparticles, which were prepared concurrently in order to minimize overall reaction time.

### Step 3: Coupling of the Free Sulfhydryl-Bearing Antibody to Maleimido-CLIO Nanoparticles

The half-IgG with available sulfhydryl groups were reacted with maleimido-CLIO to link antibody fragments covalently through thioether bonds to iron oxide nanoparticles. Specifically, half-IgG-SH (approximately 2.5 nmol in 60 μL, pH 6.5) was combined with maleimido-CLIO (0.25 nmol nanoparticles in 22.5 μL, pH 7.0).

This was allowed to react overnight at 4° C. The unbound antibody fragments were separated from nanoparticles by centrifugal size-exclusion columns packed with Sephacryl 300HR, pre-equilibrated with PBS pH 6.5 buffer.

The efficiency of coupling of the antibody fragments to the nanoparticles was determined by comparing the HPLC profile of (a) the antibody solution before coupling and (b) the recovered unbound antibody after coupling and purification. The HPLC method used a size-exclusion column (BioSilect 400) eluted with 100 mM phosphate buffer, 150 mM NaCl, pH 6.8. The flow rate was 1 mL per minute and the eluate was monitored with a Waters UV/Vis spectrophotometer at 280 nm for protein or 540 nm for Rhodamine. The areas under the peaks corresponding to antibody and gelatin were compared, before and after coupling. The area under the antibody peak declined because some antibody had bound to the nanoparticles, whereas the area under the gelatin peak did not change because gelatin does not contain cysteine amino acids and thus does not have available sulfhydryls to bind to maleimido-CLIO. The sulfhydryl group on half-IgG coupled to the maleimide group with 80% efficiency.

The resulting product contained an average of 8 half-IgGs coupled to each CLIO bead and is tagged with Rhodamine. The product will be referred to hereafter as Rhodamine-CLIO-pAb416.

### Example 6

Uptake of Rhodamine-CLIO-pAb416 Nanoparticles by T-Antigen Expressing Medulloblastoma Cells

BSB8 mouse medulloblastoma cells, which are transformed by JC virus T-antigen and express T-antigen in the nucleus, were seeded at 2×10⁴ cells per well in Permanox chamber slides and were cultured for 2 days in Dulbecco's modified Eagle's medium (DMEM) with 10% fetal bovine serum in a humidified incubator at 37° C. with 5% CO₂. The cells were adherent to the slides at this time. Then the media was aspirated and replaced with fresh media containing Rhodamine-CLIO-pAb416. The cells were incubated with Rhodamine-CLIO-pAb416, and fixed with 4% paraformaldehyde after incubation for 2 or 24 h. As controls, to other wells were added (a) CLIO-Rhodamine (nanoparticle derivative prepared by reacting NHS-Rhodamine with CLIO-NH₂), or (b) Rhodamine-pAb416 (antibody coupled to Rhodamine but not coupled to CLIO). Slides were mounted with DAPI to stain cell nuclei and then examined by fluorescence microscopy (DAPI or 4′,6-diamidino-2-phenylindole is a fluorescent stain that binds strongly to DNA. Since DAPI passes through an intact cell membrane, it may be used to stain both live and fixed cells.).

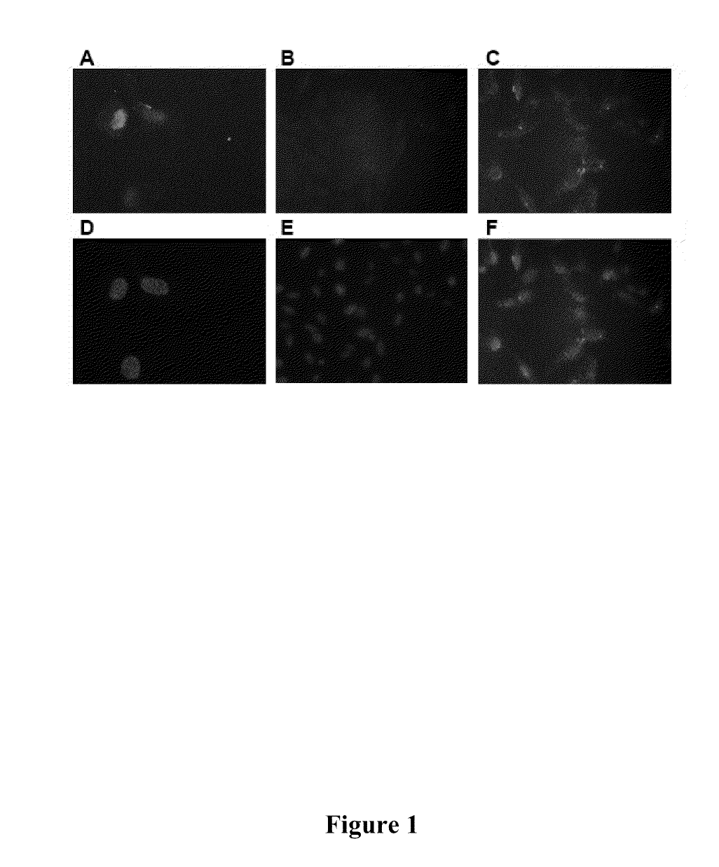

FIG. 1 summarizes the results of the experiment. The anti-T-antigen antibody pAb416 was selectively taken up by T-antigen expressing tumor cells when bound to CLIO (Panel A), as well as when not bound to CLIO (Panel C). On the other hand, CLIO not bound to pAb416 did not enter the cells (Panel B). Furthermore, pAb416 could be detected in a speckled pattern localized to the cytoplasm of the cells after incubation for 2 h (Panel C) while CLIO-pAb416 accumulated in the nucleus (Panel A), where T-antigen is expressed, after incubation for 24 h.

### Example 7

### Specific Uptake of Nanoconjugates by T-Antigen Expressing Cells

Anti-T-antigen antibody (pAb416) and non-specific mouse (NSM) antibodies were separately covalently coupled to CLIO by thioether linkages, according to the following steps, which were discussed in details in previous examples.

First, the antibodies (pAb416 and NSM) were radiolabeled with ¹²⁵I by the Iodogen™ method. Iodogen™ labeling (Pierce) is a well-known solid-phase oxidative iodination procedure that uses 1,3,4,6-tetrachloro-3α,6α-diphenylglucoluril and has been employed to label a variety of proteins and peptides such as albumins, globulins, neuropeptides, and chemokines (Salacinzki et al., 1981, Anal. Biochem. 117:136, incorporated herein by reference). The method of Chizzonite (Chizzonite et al., 1991, J. Immunol. 147:1548-1556) was used: the radioiodide in buffer was placed into an Iodogen-coated tube for a few minutes, after which the radioiodine solution was withdrawn from the tube and transferred to a second tube containing the protein to be labeled.

Second, the ¹²⁵I-labeled antibodies were converted to half-IgG with available sulfhydryl groups by reaction with 2-mercaptoethylamine and subsequent purification. Third, CLIO-NH₂ was converted to maleimido-CLIO by reaction with sulfo-SMCC and subsequent purification. Fourth, coupling reactions between maleimido-CLIO and ¹²⁵I-labeled half-IgGs were then set up with various molar ratios of reagents as shown in Table 1.

TABLE 1

| | ¹²⁵I-pAb416-SH* | ¹²⁵I-NSM-SH* | CLIO-maleimide |
|---|---|---|---|
| Reaction | pmol | pmol | pmol |
| A | 150 | 0 | 15 |
| B | 112.5 | 37.5 | 15 |
| C | 75 | 75 | 15 |
| D | 37.5 | 112.5 | 15 |
| E | 0 | 150 | 15 |

*pmol of half-IgG fragments

Each reaction was allowed to proceed overnight at 4° C. before removal of unbound antibody by centrifugal size-exclusion column packed with Sephacryl 300HR. Recovery of ¹²⁵I indicated that approximately 80% of added antibody was bound to CLIO.

To test the number of antibody fragments needed to achieve targeting, conjugates were prepared with 2, 4, 6 or 8 antibody fragments (half-IgG) per CLIO. The number of fragments per CLIO was adjusted to a total of 8 in each case by making up the difference with nonspecific antibody (NSM).

In the reactions described above, Reaction A resulted in 8 pAb416/CLIO, Reaction B resulted in 6 pAb416 and 2 NSM per CLIO, Reaction C resulted in 4 pAb416 and 4 NSM of each antibody fragment per CLIO, Reaction D resulted in 2 pAb416 and 6 NSM per CLIO, and Reaction E resulted in 8 NSM per CLIO.

T-Antigen positive mouse medulloblastoma cells were seeded in 24-well plates at 10⁵ cells per well and cultured for 2 days (37° C., 5% CO₂). The media was removed from each well by aspiration. Radiolabeled conjugates derived from reactions A-E above, diluted in culture media, were added to the cells (0.5 ml per well) and were incubated (37° C., 5% CO₂) for 1, 2, 6 or 24 hours. The concentration of conjugates placed in each well was adjusted to 1 nM total antibody to equalize the concentration across wells. As controls, radiolabeled pAb416 or NSM IgG (not coupled to nanoparticles) were tested in additional wells. After incubation, unbound conjugates were aspirated and cells were washed with 0.5 ml buffer. The wash was added to the initial aspirate and termed “unbound” conjugate. Surface-bound conjugate was determined by incubating cells with 0.5 ml of 0.5 M NaCl, 0.2 M acetic acid at 4° C. for 5 min. This solution, and a subsequent 0.5 ml PBS wash, were combined and termed “surface bound” conjugate. Finally, the washed cells were removed from wells by detaching with 0.5 ml of 0.05% trypsin, 0.5 mM EDTA for 5 min. The cells were collected and combined with a 0.5 ml PBS wash of the well and termed “internalized” conjugates. Unbound, surface-bound and cells with internalized conjugates were counted separately. This experiment was performed in triplicate (3 wells for each different conjugate).

The percentage of internalized conjugate was calculated using the formula: [100×“internalized”/(“unbound”+“surface bound”+“internalized”)].

The total percentage of conjugates bound to cells was calculated using the formula: [100×(“surface bound”+“internalized”)/(“unbound”+“surface bound”+“internalized”)].

The specificity of uptake was tested in one experiment by adding 100 fold excess unconjugated pAb416 along with ¹²⁵I-Ab-CLIO. This was also performed in triplicate. After 1 hour of incubation, the wells were processed to separate the unbound, surface bound and internalized fractions, as described above.

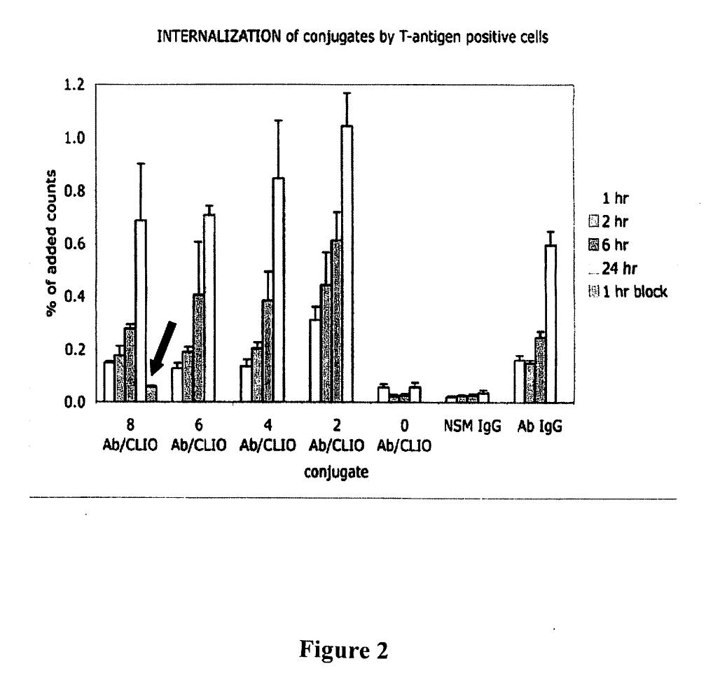

FIG. 2 summarizes the levels of internalization of conjugates by T-antigen positive cells, for pAb416-CLIO conjugates ranging from 0 to 8 pAb416 antibodies per nanoparticle. All conjugates containing targeting antibody pAb416 (2, 4, 6 or 8 pAb416 antibodies per nanoparticle) were found to bind to cells and become internalized, with increasing levels over time. In fact, there was no significant difference in cell binding or internalization between conjugates containing 2, 4, 6 or 8 Ab fragments per CLIO. As expected, the conjugate containing only NSM antibody (0 Ab/CLIO conjugate) and the NSM IgG itself were not internalized at any time point. The non-conjugated Ab IgG also showed internalization under the reaction condition but to a lower level. Finally, the control reaction, where 100-fold excess of Ab IgG was added to the reaction mixture containing 8 Ab/CLIO conjugate, showed that binding and internalization of ¹²⁵I-Ab-CLIO had been decreased by >80%, indicating an antigen-specific interaction.

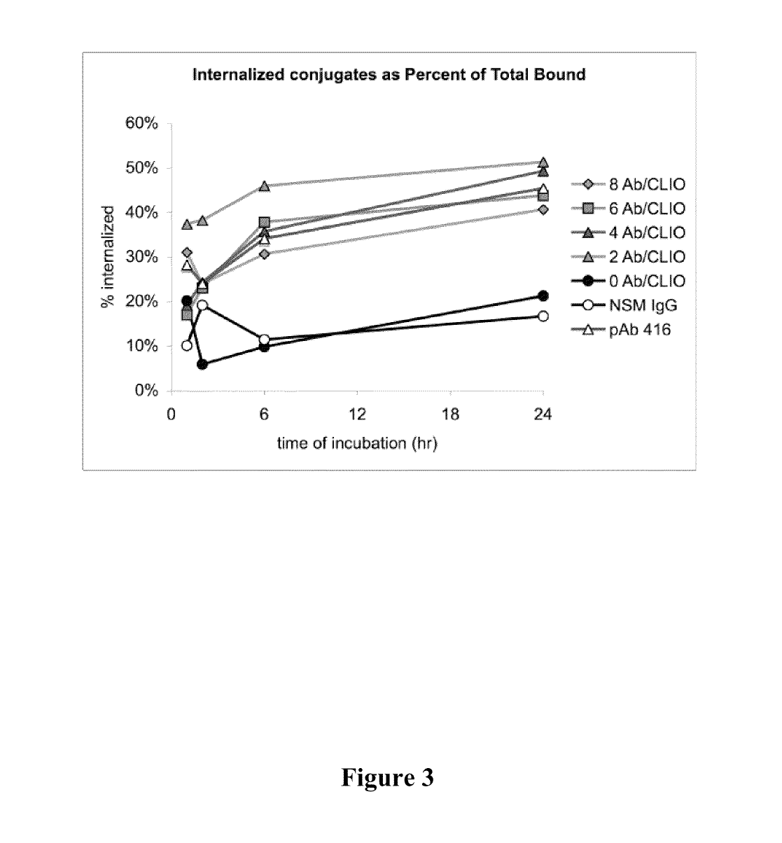

FIG. 3 depicts the level of internalized conjugate as a percentage of total bound conjugate, for pAb416-CLIO conjugates ranging from 0 to 8 pAb416 antibodies per nanoparticle. As controls, experiments were also run with NSM-CLIO and non-conjugated pAb416. For all pAb416-containing conjugates, the internalized counts (internalized conjugate) represented 41-51% of total counts (bound conjugate) at the 24 hour time point. Conjugates with only NSM antibody on CLIO, or unconjugated NSM antibody, had significantly lower total binding and internalization than conjugates with Ab. Unconjugated ¹²⁵I-Ab antibody had equivalent or slightly lower cell uptake compared with targeted CLIO conjugates.

### Example 8

### Evaluation of Possible Cytotoxic Effects of Ab-CLIO on T-Antigen Positive Cells

The iron loading and cytotoxic effects of Ab-CLIO on T-antigen positive cells were tested. Various concentrations of Ab-CLIO conjugate (measured as 5, 25, 50, 100, 150, 200, 300 ug iron/mL in media, or media alone with no conjugates) were added to growing T-antigen positive cells in CC2-coated glass chamber slides. The slides were then incubated for 4 or 24 hours further, and examined by phase microscopy, using Trypan blue to check for dead cells. The cells were then fixed and the iron in the conjugates was stained with Perls's Prussian blue method (Perls, 1867, Arch. Pathol 39, 42) and the slides were evaluated by light microscopy.

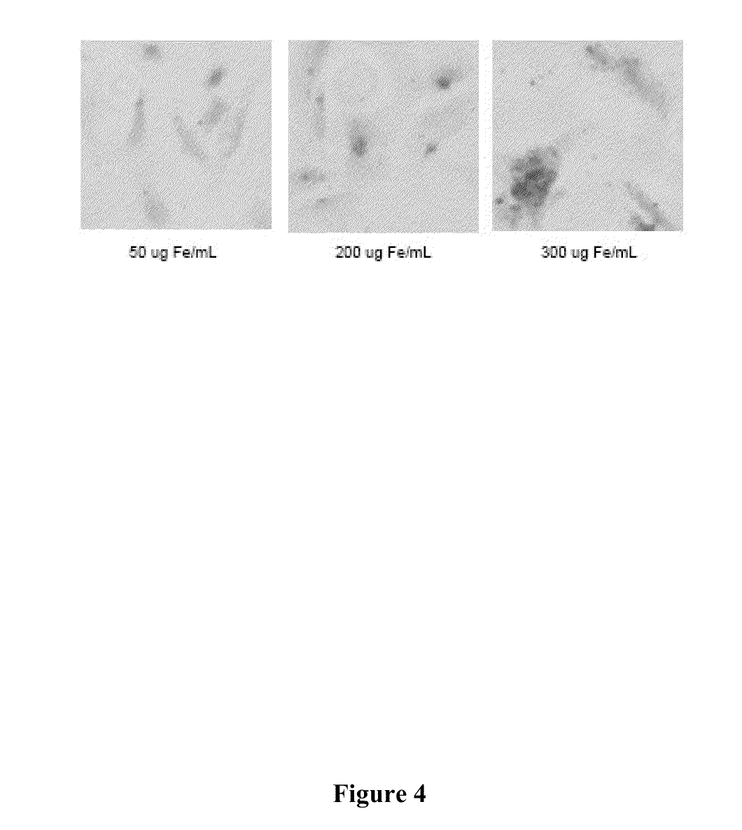

FIG. 4 displays the staining of cells with Perls' Prussian Blue after staining with varying levels of Ab-CLIO. Increasing concentrations of Ab-CLIO resulted in increasing amounts of iron inside cells. At the highest iron levels there was only a small effect on cell morphology. After 4 hr of incubation, only the cells incubated with 300 μg Fe/mL showed any sign of rounding up. These cells were heavily loaded with iron, as seen in the Prussian blue stained images in FIG. 4. There were no appreciable morphologic changes at lower iron loadings.

Viability was evaluated using uptake of Trypan Blue, a dye only absorbed by dead cells or tissues. After 24 hr incubation, exposure to 100 μg Fe/mL (the highest level tested for 24 hr) resulted in 4% of cells taking up Trypan blue, compared with only 1% of cells exposed to 25 μg Fe/ml or media only.

This experiment further confirms that iron nanoparticles are being delivered to the cells. No iron accumulation was observed for conjugates without targeting antibody (not shown).

### Example 9

### Labeling of Nanoparticules With Membrane Penetration Peptide

In order to label a nanoparticle with the membrane penetration peptide Tat, nanoparticles CLIO-NH₂ (0.5 nmol particles, 57.6 μg Fe, suspended in 30 uL bicarbonate buffer, pH 8.0) were diluted with 15 μl 0.1M phosphate, pH 7.4, treated with sulfo-SMCC (50 nmol in 5 μl in water) and allowed to react for 2 hr at room temperature. After reacting for 2 hr at room temperature, the nanoparticles are purified using a centrifugal size exclusion column (BioGel P-6, pre-equilibrated in 0.1M phosphate, pH 7.0), affording maleimido-CLIO.

Maleimido-CLIO was then coupled with the free sulfhydryl group on the Fl-Tat peptide. Fl-Tat peptide was dissolved to 4.4 mg/ml in 20 mM citrate, 5 mM EDTA, pH 6.5, and sparged with argon gas to remove dissolved oxygen. A 15-fold molar ratio of Fl-Tat peptide was slowly added to maleimido-CLIO, and the reaction was held at 4° C. overnight. Unbound peptide was separated from the nanoparticles by using appropriate size-exclusion spin columns. On average, depending on the reaction conditions and reaction contact time, 2-12 Tat peptides were attached on average to each CLIO particles.

In order to label a nanoparticle with both Fl-Tat peptide and Rhodamine-labeled pAb416, maleimido-CLIO was reacted with a mixture of Fl-Tat peptide and Rhodamine-labeled half IgG. A 15-fold molar ratio of Fl-Tat peptide and a 10-fold molar ratio of Rhodamine-labeled half IgG were combined with maleimido-CLIO. After reacting 60 hours at 4° C., the conjugated CLIO was separated from the lower molecular weight unbound molecules by by centrifugal size-exclusion column packed with Sephacryl 300HR. The CLIO nanoparticles labeled with both Fl-Tat and pAb416 are referred to as Tat-pAb416-CLIO.

### Example 10

### Internalization of Antibody-Conjugated Nanoparticles (Part 1)

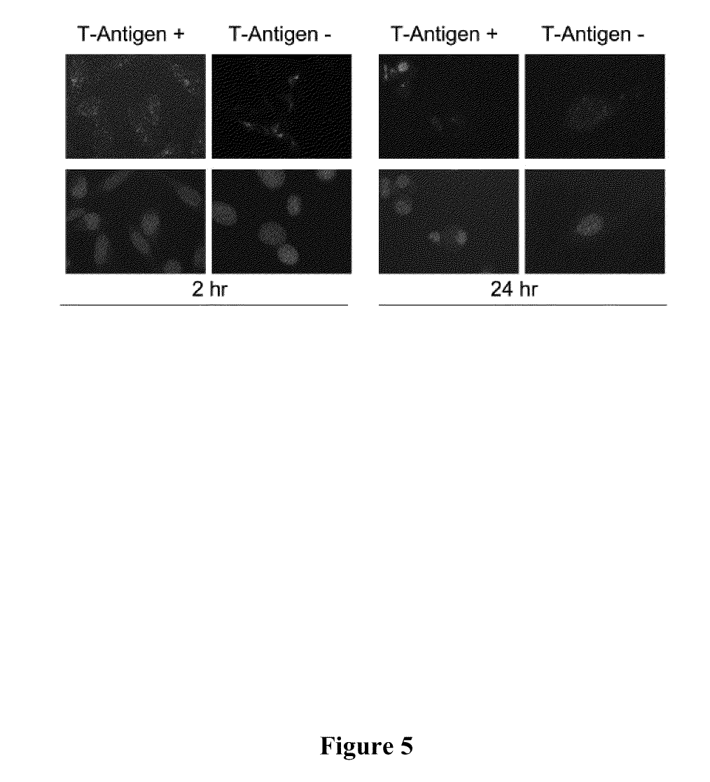

In the following example, the Tat-pAb416-CLIO used contained an average of 6.3 pAb416 and 7.2 Tat units per CLIO. T-antigen positive and T-antigen negative cells were treated with Tat-pAb416-CLIO nanoparticles for 2 and 24 hours, as shown in FIG. 5.

Uptake of the nanoparticles by T-antigen negative cells was very slight, demonstrating that the Tat peptide does not cause non-specific internalization. On the other hand, Tat-pAb416-CLIO and pAb416-CLIO (lacking the Tat peptide) appeared to undergo internalization in similar levels in T-antigen positive cells. The qualitative study suggested that the presence of the Tat peptide together with pAb416 on the nanoparticle does not provide much of an advantage over the pAb416 coupled to the nanoparticle in terms of cell penetration (internalization). In fact, pAb416-CLIO even underwent nuclear localization even in the absence of the Tat peptide, as evidenced in the bottom row of FIG. 5.

As expected, Rhodamine-CLIO, lacking targeting antibody or Tat, did not bind or enter cells (data not shown), and non-specific mouse antibody, alone or bound to CLIO, did not bind to cells (data not shown).

### Example 11

### Internalization of Antibody-Conjugated Nanoparticles (Part 2)

The results in Example 10 were further explored by preparing a series of CLIO nanoparticles displaying different rates of ¹²⁵I-pAb416 antibody and Tat peptide, as shown in Table 2. These CLIO nanoparticles were then tested for internalization by T-antigen positive cells.

TABLE 2

| Conjugate ID | Ab per CLIO | Tat per CLIO |
|---|---|---|
| ¹²⁵I-Ab-CLIO | 2 | 0 |
| Ab:Tat 2:0 | | |
| ¹²⁵I-Ab-Tat-CLIO | 2 | 2 |
| Ab:Tat 2:2 | | |
| ¹²⁵I-Ab-Tat-CLIO | 2 | 6 |
| Ab:Tat 2:6 | | |
| ¹²⁵I-Ab-Tat-CLIO | 8 | 2 |
| Ab:Tat 8:2 | | |

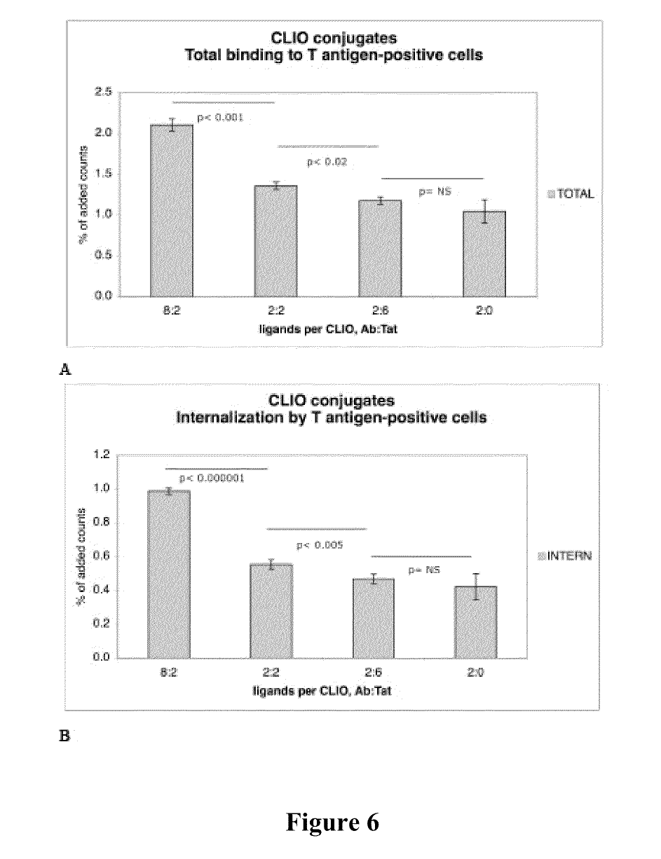

The CLIO conjugates were incubated with T-antigen positive cells for 24 hours, then the cells were processed to separate unbound and surface-bound conjugates from internalized conjugates as in Example 10. The results are shown in FIG. 6. The experiments indicated that at a level of 2 Ab fragments per CLIO particle, the presence of Tat peptide provides at most a slight enhancement of cell uptake. A larger effect was seen when 8 Ab fragments were combined with 2 Tat fragments; this resulted in a doubling of the cell binding and internalization compared with 2 Ab fragments and no Tat. The relative number of Ab fragments and Tat peptides on the nanoparticle may be adjusted to achieve optimal internalization.

### Example 12

### Conjugation of Radiometal Chelate to CLIO

In a typical labeling protocol, amino-containing nanoparticles, such as CLIO-NH₂, are reacted with the sulfosuccinimide ester of 1,4,7,10-tetraazacyclododecane-N,N′,N″,N′″-tetraacetic acid, also known as DOTA (Lewis et al., 2001, Bioconj. Chem. 12:320-324), or the sulfosuccinimide ester of diethylene triamine pentaacetic acid, also known as DTPA (Wunderbaldinger et al., 2002, Bioconj. Chem. 13:264-268). The nanoparticles labeled with the chelating element may then purified by centrifugal size exclusion column.

¹¹¹In chloride in dilute HCl is buffered by addition of acetate or citrate buffer, pH 5.7, prior to adding to the nanoparticles labeled with the chelating element (Knight et al., 1987, Biochim. Biophys. Acta. 924:45-53; Lewis et al., 2001, Bioconj. Chem. 12:320-324). The labeled particles are purified by PD-10 column equilibrated in a buffer suitable for the assay (in vitro or in vivo).

### Example 13

### Generation of Antibody-Immobilized Nanoparticles

Maleimido-CLIO nanoparticles may be reacted with pAb416, a mouse monoclonal antibody that recognizes JCV as well as SV40 and BKV T-antigens, and pAb419, a mouse monoclonal antibody that recognizes SV40 and BKV T-antigens but not JCV T-antigen. These antibodies are utilized for immunohistochemistry, immunoprecipitation and Western blotting, and recognize conformation-independent epitopes. In order to couple an antibody to the maleimido-modified nanoparticles, a free and accessible sulfhydryl group needs to be present on the antibody. The two most common techniques to achieve this goal are: (a) direct reduction of internal disulfides with 2-mercaptoethylamine (MEA), and (b) conversion of a primary amine group to a sulfhydryl group by coupling with a modifying agent such as N-succinimidyl S-acetylthioacetate (SATA). Technique (a) was described in Example 6 and selectively reduces disulfide groups in the hinge region of IgG, which is not likely to be near the antigen-binding region. Excess MEA is removed by passing the reduced IgG through a P-6 gel centrifugation column equilibrated with PBS pH 6.5, 1 mM EDTA. The product is immediately used for coupling to modified nanoparticles. The sulfhydryl modified antibody is added to maleimido-modified CLIO and allowed to react for 3 hr at room temperature. Unbound antibody is removed on a centrifuged column. Prior to conjugation, the IgG may be tagged with a fluorescent label such as NHS-Rhodamine. The number of attached antibodies per nanoparticle is determined using the fluorescent tags on each substituent to quantify the concentration of attached molecules (Wunderbaldinger et al., 2002, Bioconj. Chem. 13:264-268), and a spectrophotometric method to determine the CLIO concentration based on its iron content (Moore et al., 2001, Radiology. 221:244-250).

In some studies, separate radiolabels may be used to track the iron oxide nanoparticle (labeled with ¹¹¹In as described above) and the antibody fragment attached to it, to determine whether and for how long they remain together. For radiolabeling the antibody fragment, ¹²⁵I labeling may be used. The antibody is labeled directly with ¹²⁵I or via an indirect iodination technique using a radiolabeled agent such as N-succinimidyl-para-[¹²⁵I]-iodobenzoate. The later technique should result in a non-metabolizable iodine label that remains associated with the antibody rather than rapidly dissociating and washing out of cells.

### Example 14

### Characterization of Nanoconjugates

The size of the nanoconjugates prepared in Example 13 may be measured using a laser-based Malvern Zetasizer 1000HAS spectrometer (Malvern Instruments). The number of attached antibodies and Tat peptides per nanoparticle is determined using the fluorescent tags on each substituent to quantify the concentration of attached molecules, and a spectrophotometric method may be used to determine the CLIO concentration based on its iron content. The nanoparticles generally contain 2064 atoms of iron per particle (Moore et al., 2001, Radiology 221:24-250). Coupling ratios may be altered based on subsequent experiments, to optimize targeting and internalization of nanoparticles into tumor cells.

### Example 15

### Testing Stability and Intracellular Location of Nanoconjugates In Vitro

The methods described herein allow for the study of localization of nanoconjugates to the cell nucleus after binding and internalization. If detectable amounts of the nanoconjugates are internalized by the T-antigen expressing cells but are found to be retained outside the nucleus (i.e. in the cytoplasm or membrane fractions), it is possible to determine whether the nanoconjugates are associated with T-antigen in these extranuclear locations.

T-antigen positive cells may be grown in tissue culture flasks. Nanoconjugates containing anti-T-antigen antibody (labeled with fluorescent and/or radioactive reporters) are incubated with the cells in culture, then unbound nanoconjugates are removed and the cells are then incubated for various times (e.g. 2, 6, 24 and 48 hours). Subcellular localization of the nanoconjugates is determined in parallel by microscopy (immunofluorescence and iron oxide staining) and by subcellular fractionation of nuclear vs. cytoplasmic fractions, followed by quantitation of proteins by radiolabeling and by Western blotting. If detectable amounts of nanoconjugates are found within the cytoplasm, electron microscopy of cell pellets may be employed to examine whether particles are localized within endosomes or within the cytosol.

### Example 16

### Stability Studies in Serum In Vitro

In order to study the stability of nanoconjugates in serum, nanoconjugates may be incubated with mouse and/or human serum in vitro at 37° C. At various times up to 48 hours, samples are removed for analysis of the integrity of the nanoconjugate. Using size exclusion spin columns, any radiolabeled antibody that has become unbound from the nanoparticle is separated and quantified.

### Example 17

### Blood Kinetics and Biodistribution of Nanoparticles in Normal Mice

To determine the distribution of the nanoparticles in vivo and to estimate the approximate dosage for subsequent imaging, studies may be performed in normal mice.

For these studies, nanoconjugates are labeled with ¹¹¹In via DOTA chelator attached to the dextran coat of the nanoparticle. After intravenous injection of labeled nanoparticles, groups of 3-5 mice are euthanized at 6, 12, 24, and 48 h post injection. Unmodified dextran-coated iron oxide nanoparticles have been reported to have a blood half-life of 10.9 hr in mice (Wunderbaldinger et al., 2002, Acad. Radiology 9 Suppl 2:S304-S306), suggesting that delivery to tissues may continue for an extended period of time after administration. Therefore, the mice should be monitored at time points up to several half-lives (e.g. 48 hr). Major organs (blood, lungs, liver, spleen, heart, kidneys, stomach, intestines, bone, muscle, thymus and brain) are sampled, weighed and counted in a gamma counter, along with a standard of the administered dose. Cage bedding is collected and counted to estimate the amount of radiolabeled nanoconjugate that has been excreted. These measurements allow the calculation of the percent of administered dose that accumulates in each tissue as a function of time after injection. If the half-life of the particles in vivo is long (e.g., nanoparticle are still present at 48 h post injection), additional time points for monitoring may be required.

At several time points prior to euthanasia, blood is sampled retroorbitally for determination of blood disappearance rate. The samples of blood are weighed and counted in a gamma counter relative to a standard of the administered dose, in order to determine the percentage of the administered dose in the blood at various times after injection.

The blood clearance data and the time course of biodistribution are used to guide the timing of experiments with nude mice bearing tumor xenografts.

### Example 18

### Specificity of Nanoparticle Tumor Targeting in the Nude Mouse Tumor Model

In addition to normal mice, the nude mouse flank model may be utilized for initial characterization of the behavior of the nanoparticles and their trafficking to T-antigen expressing tumor cells. With that objective, nude mice are utilized for transplantation of T-antigen positive and T-antigen negative mouse cell lines. Approximately 1×10⁶ to 1×10⁷ cells from lines derived from T-antigen positive transgenic animal tumors are injected subcutaneously (into the flank) with a 26 gauge needle in a volume of up to 0.1 ml with a Hamilton syringe. T-antigen negative cells are injected into the flank of control animals in parallel, as a control for nanoparticle specificity.

Following injection, animals are randomly separated into control and treatment groups, as needed. Growth of the tumor is measured weekly with a caliper (mm scale). When tumors reach approximately 0.5 cm in diameter, animals are injected with nanoparticle formulations followed by euthanasia and histopathological correlation.

### Example 19

### Pathological Analysis and Detection of Nanoparticles in Animal Tissues

In general, necropsy, tissue accessioning, routine histology and immunohistochemistry may be performed in animal tissues, following nanoparticle administration. Animals are perfusion-fixed and the morphological evaluations are performed on their tissue. Tissue for histological analysis is dehydrated through a graded series of ethanol before being embedded in paraffin. Immunofluorescence of formalin-fixed paraffin embedded sections is performed to detect the fluorescent-tagged antibody. In addition, immunohistochemistry is performed using antibodies that recognize T-antigen or any other antigen of choice.

Briefly, tissue sections are deparaffinized, rehydrated, and quenched for endogenous peroxidase in 3% H₂O₂/methanol for 15 min at room temperature. If necessary, antigen retrieval is performed in 0.01 M sodium citrate buffer, pH 6.0 for 30 min at 95° C. Samples are then be incubated in primary antibody overnight at 4° C., followed by detection using the avidin-biotin complex (ABC) technique with diaminobenzidine (DAB) as the chromagen (Vectastain Elite Kit, Vector Labs). Negative controls consist of normal sera in the place of primary antibody. A minimum of five individual sets of age-matched and induction matched tumor samples should be prepared from each timepoint for histological analysis. Hematoxylin and eosin and iron staining (Prussian blue) are performed on parallel sections of tumor. Serial sections of lesions or tumor foci are stained using antibodies to structural proteins as needed.

In terms of slide analysis, a minimum of 10 non-overlapping high magnification fields (40×) are counted for each specimen and for each antibody or stain. Slides are blindly scored on the basis of staining intensity and subcellular localization. Deconvolution fluorescence microscopy is utilized in order to co-localize the nanoparticle core and the antibody fragment.

The disclosures of each and every patent, patent application, and publication cited herein are hereby incorporated herein by reference in their entirety. One skilled in the art will readily appreciate that the present invention is well adapted to carry out the objects and obtain the ends and advantages mentioned, as well as those inherent therein. While the invention has been disclosed with reference to specific embodiments, it is apparent that other embodiments and variations of this invention may be devised by others skilled in the art without departing from the true spirit and scope used in the practice of the invention. The appended claims are intended to be construed to include all such embodiments and equivalent variations.

What is claimed:

1. A construct comprising a targeting member immobilized on a detectable particulate, wherein said detectable particulate has an intrinsic property that allows for monitoring, detection or imaging in vivo or in vitro, wherein binding between said targeting member and a target structure that is preferentially expressed on the surface of a cancer cell induces internalization of said construct by said cancer cell.

2. The construct of claim 1, wherein said detectable particulate ranges in size from about 1 nm to about 100 nm in its medium dimension.

3. The construct of claim 2, wherein said detectable particulate ranges in size from about 3 nm to about 50 nm in its medium dimension.

4. The construct of claim 1, wherein said detectable particulate comprises monocrystalline iron oxide nanoparticles.

5. The construct of claim 4, wherein said nanoparticles are covered with a coating.

6. The construct of claim 5, wherein said coating is dextran.

7. The construct of claim 5, wherein said coating is cross-linked dextran.

8. The construct of claim 5, wherein said coating is polyethylene glycol.

9. The construct of claim 1, wherein said targeting member comprises an antibody that binds selectively to said target structure.

10. The construct of claim 9, wherein said antibody is polyclonal.

11. The construct of claim 9, wherein said antibody is chimeric.

12. The construct of claim 11, wherein said antibody is humanized.

13. The construct of claim 1, wherein said target structure comprises human polyomavirus T-antigen.

14. The construct of claim 1, wherein said targeting member comprises an antibody that binds selectively to human polyomavirus T-antigen.

15. The construct of claim 1, where said target structure comprises CD7, CD9, CD22, CD25, CD30, CD33, CD56, Le^{y}, TFR, EGFR, ErbB2, IL-4R, IL-13R or mesothelin.

16. The construct of claim 1, wherein said targeting member comprises an antibody that binds selectively to CD7, CD9, CD22, CD25, CD30, CD33, CD56, Le^{y}, TFR, EGFR, ErbB2, IL-4R, IL-13R or mesothelin.

17. The construct of claim 1, further comprising a blood-brain barrier penetration element immobilized on said detectable particulate.

18. The construct of claim 17, wherein said blood-brain barrier penetration element comprises insulin (SEQ ID NO:15), antibodies against the human insulin receptor, Thr-Phe-Phe-Tyr-Gly-Gly-Cys-Arg-Gly-Lys-Arg-Asn-Asn-Phe-Lys-Thr-Glu-Glu-Tyr (SEQ ID NO:16), Thr-Phe-Phe-Tyr-Gly-Gly-Ser-Arg-Gly-Lys-Arg-Asn-Asn-Phe-Lys-Thr-Glu-Glu-Tyr (SEQ ID NO:17), Thr-Phe-Phe-Tyr-Gly-Gly-Ser-Arg-Gly-Lys-Arg-Asn-Asn-Phe-Arg-Thr-Glu-Glu-Tyr (SEQ ID NO:18), Thr-Phe-Phe-Tyr-Gly-Gly-S er-Arg-Gly-Arg-Arg-Asn-Asn-Phe-Arg-Thr-Glu-Glu-Tyr (SEQ ID NO:19), Thr-Phe-Phe-Tyr-Gly-Gly-Cys-Arg-Ala-Lys-Arg-Asn-Asn-Phe-Lys-Arg-Ala-Lys-Tyr (SEQ ID NO:20), Thr-Phe-Phe-Tyr-Gly-Gly-Cys-Arg-Gly-Lys-Lys-Asn-Asn-Phe-Lys-Arg-Ala-Lys-Tyr (SEQ ID NO:21), Pro-Phe-Phe-Tyr-Gly-Gly-Cys-Arg-Gly-Lys-Arg-Asn-Asn-Phe-Lys-Thr-Glu-Glu-Tyr (SEQ ID NO:22), Thr-Phe-Phe-Tyr-Gly-Gly-Cys-Arg-Gly-Lys-Arg-Asn-Asn-Phe-Lys-Thr-Lys-Glu-Tyr (SEQ ID NO:23), Thr-Phe-Phe-Tyr-Gly-Gly-Lys-Arg-Gly-Lys-Arg-Asn-Asn-Phe-Lys-Thr-Lys-Glu-Tyr (SEQ ID NO:24), Thr-Phe-Phe-Tyr-Gly-Gly-Cys-Arg-Gly-Lys-Arg-Asn-Asn-Phe-Lys-Thr-Lys-Arg-Tyr (SEQ ID NO:25), Thr-Phe-Phe-Tyr-Gly-Gly-Lys-Arg-Gly-Lys-Arg-Asn-Asn-Phe-Lys-Thr-Ala-Glu-Tyr (SEQ ID NO:26), Thr-Phe-Phe-Tyr-Gly-Gly-Lys-Arg-Gly-Lys-Arg-Asn-Asn-Phe-Lys-Arg-Glu-Lys-Tyr (SEQ ID NO:27), Arg-Phe-Lys-Tyr-Gly-Gly-Cys-Leu-Gly-Asn-Lys-Asn-Asn-Phe-Leu-Arg-Leu-Lys-Tyr (SEQ ID NO:28), or Arg-Phe-Lys-Tyr-Gly-Gly-Cys-Leu-Gly-Asn-Lys-Asn-Asn-Tyr-Leu-Arg-Leu Lys Tyr (SEQ ID NO:29).

19. The construct of claim 1, further comprising a therapeutic agent immobilized on said detectable particulate.

20. The construct of claim 19, wherein said therapeutic agent comprises one or more of vinca alkaloids, taxanes, topoisomerase inhibitors, antitumor antibiotics, plant toxins, bacterial toxins, siRNAs, miRNAs or antisense oligonucleotides.

21. The construct of claim 1, further comprising Rhodamine covalently attached to the construct.

22. A method of monitoring, detecting or imaging a cancer cell in a cell culture or a tissue, comprising exposing said cancer cell to a construct comprising a targeting member immobilized on a detectable particulate, wherein said detectable particle has an intrinsic property that allows for monitoring, detecting or imaging said cancer cell, wherein binding between said targeting member and a target structure that is preferentially expressed on the surface of said cancer cell induces internalization of said construct by said cancer cell.

23. The method of claim 22, wherein said targeting member comprises an antibody that binds selectively to said target structure.

24. The method of claim 23, wherein said target structure comprises human polyomavirus T-antigen.

25. The method of claim 22, wherein said construct further comprises a blood-brain barrier penetration element immobilized on said detectable particulate, wherein said blood-brain barrier penetration element comprises insulin (SEQ ID NO:15), antibodies against the human insulin receptor, Thr-Phe-Phe-Tyr-Gly-Gly-Cys-Arg-Gly-Lys-Arg-Asn-Asn-Phe-Lys-Thr-Glu-Glu-Tyr (SEQ ID NO:16), Thr-Phe-Phe-Tyr-Gly-Gly-Ser-Arg-Gly-Lys-Arg-Asn-Asn-Phe-Lys-Thr-Glu-Glu-Tyr (SEQ ID NO:17), Thr-Phe-Phe-Tyr-Gly-Gly-Ser-Arg-Gly-Lys-Arg-Asn-Asn-Phe-Arg-Thr-Glu-Glu-Tyr (SEQ ID NO:18), Thr-Phe-Phe-Tyr-Gly-Gly-Ser-Arg-Gly-Arg-Arg-Asn-Asn-Phe-Arg-Thr-Glu-Glu-Tyr (SEQ ID NO:19), Thr-Phe-Phe-Tyr-Gly-Gly-Cys-Arg-Ala-Lys-Arg-Asn-Asn-Phe-Lys-Arg-Ala-Lys-Tyr (SEQ ID NO:20), Thr-Phe-Phe-Tyr-Gly-Gly-Cys-Arg-Gly-Lys-Lys-Asn-Asn-Phe-Lys-Arg-Ala-Lys-Tyr (SEQ ID NO:21), Pro-Phe-Phe-Tyr-Gly-Gly-Cys-Arg-Gly-Lys-Arg-Asn-Asn-Phe-Lys-Thr-Glu-Glu-Tyr (SEQ ID NO:22), Thr-Phe-Phe-Tyr-Gly-Gly-Cys-Arg-Gly-Lys-Arg-Asn-Asn-Phe-Lys-Thr-Lys-Glu-Tyr (SEQ ID NO:23), Thr-Phe-Phe-Tyr-Gly-Gly-Lys-Arg-Gly-Lys-Arg-Asn-Asn-Phe-Lys-Thr-Lys-Glu-Tyr (SEQ ID NO:24), Thr-Phe-Phe-Tyr-Gly-Gly-Cys-Arg-Gly-Lys-Arg-Asn-Asn-Phe-Lys-Thr-Lys-Arg-Tyr (SEQ ID NO:25), Thr-Phe-Phe-Tyr-Gly-Gly-Lys-Arg-Gly-Lys-Arg-Asn-Asn-Phe-Lys-Thr-Ala-Glu-Tyr (SEQ ID NO:26), Thr-Phe-Phe-Tyr-Gly-Gly-Lys-Arg-Gly-Lys-Arg-Asn-Asn-Phe-Lys-Arg-Glu-Lys-Tyr (SEQ ID NO:27), Arg-Phe-Lys-Tyr-Gly-Gly-Cys-Leu-Gly-Asn-Lys-Asn-Asn-Phe-Leu-Arg-Leu-Lys-Tyr (SEQ ID NO:28), or Arg-Phe-Lys-Tyr-Gly-Gly-Cys-Leu-Gly-Asn-Lys-Asn-Asn-Tyr-Leu-Arg-Leu Lys Tyr (SEQ ID NO:29).

26. A method of killing or preventing the growth of a cancer cell in a cell culture or a tissue, comprising exposing said cancer cell to a construct comprising a targeting member and a therapeutic agent immobilized on a detectable particulate, wherein binding between said targeting member and a target structure that is preferentially expressed on the surface of said cancer cell induces internalization of said construct by said cancer cell.

27. The method of claim 26, wherein said therapeutic agent comprises one or more of vinca alkaloids, taxanes, topoisomerase inhibitors, antitumor antibiotics, plant toxins, bacterial toxins, or siRNA.

28. A method of killing or preventing the growth of cancer cells in a subject in need thereof, the method comprising administering to said subject a therapeutically effective amount of a pharmaceutical composition comprising a construct comprising a targeting member and a therapeutic agent immobilized on a detectable particulate, wherein binding between said targeting member and a target structure that is preferentially expressed on the surface of said cancer cells induces internalization of said construct by said cancer cells.

29. The method of claim 28, wherein said pharmaceutical composition is administered locally to one or more sites on said subject wherein said cancer cells are located.

30. The method of claim 28, wherein said subject is human.
